SEQUENCE LISTING (68 sequences)

SEQ ID NO: 1 - protein, 225 aa, Homo sapiens
MDQQAIYAELNLPTDSGPESSSPSSLPRDVCQGSPWHQFALKLSCAGIILLVLVVTGLSVSVTSLIQKSSIEKCSVDIQQSRNKTTERPGLLNCPIYWQQLREKCLLFSHTVNPWNNSLADCSTKESSLLLIRDKDELIHTQNLIRDKAILFWIGLNFSLSEKNWKWINGSFLNSNDLEIRGDAKENSCISISQTSVYSEYCSTEIRWICQKELTPVRNKVYPDS

SEQ ID NO: 2 - protein, 191 aa, Homo sapiens
MHDSNNVEKDITPSELPANPGCLHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 3 - protein, 5 aa, synthetic construct
HYAIA

SEQ ID NO: 4 - protein, 17 aa, synthetic construct
GIIPEFGNANYAQKFQG

SEQ ID NO: 5 - protein, 14 aa, synthetic construct
EGLYYRTSNYGMDV

SEQ ID NO: 6 - protein, 11 aa, synthetic construct
RASQSVSSNLA

SEQ ID NO: 7 - protein, 7 aa, synthetic construct
GASTRVT

SEQ ID NO: 8 - protein, 9 aa, synthetic construct
QQLSPWPWT

SEQ ID NO: 9 - protein, 123 aa, synthetic construct
QVQLVQSGAEVKKPGSSVKVSCKASGGTFSHYAIAWVRQAPGQGLEWMGGIIPEFGNANYAQKFQGRVTITADESTSTAYMELSSLRSEDTAVYYCAREGLYYRTSNYGMDVWGQGTTVTVSS

SEQ ID NO: 10 - protein, 107 aa, synthetic construct
EIVLTQSPATLSVSPGERATLSCRASQSVSSNLAWYQQKPGQAPRLLIYGASTRVTGIPARFSGSGSGTEFTLTISSLQSEDFAVYYCQQLSPWPWTFGGGTKVEIK

SEQ ID NO: 11 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 12 - protein, 12 aa, synthetic construct
EPKSCDKTHTCP

SEQ ID NO: 13 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 14 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 106 aa, synthetic construct [sequence omitted]

SEQ ID NO: 16 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 330 aa, synthetic construct [sequence omitted]

SEQ ID NO: 23 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 25 - protein, 12 aa, synthetic construct
ESKYGPPCPSCP

SEQ ID NO: 26 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 27 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 107 aa, synthetic construct [sequence omitted]

SEQ ID NO: 38 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 39 - protein, 4 aa, synthetic construct
GGGS

SEQ ID NO: 40 - protein, 8 aa, synthetic construct
GGGSGGGS

SEQ ID NO: 41 - protein, 12 aa, synthetic construct
GGGSGGGSGGGS

SEQ ID NO: 42 - protein, 16 aa, synthetic construct
GGGSGGGSGGGSGGGS

SEQ ID NO: 43 - protein, 5 aa, synthetic construct
GGGGS

SEQ ID NO: 44 - protein, 10 aa, synthetic construct
GGGGSGGGGS

SEQ ID NO: 45 - protein, 15 aa, synthetic construct
GGGGSGGGGSGGGGS

SEQ ID NO: 46 - protein, 20 aa, synthetic construct
GGGGSGGGGSGGGGSGGGGS

SEQ ID NO: 47 - protein, 8 aa, synthetic construct
GGGGGGGS

SEQ ID NO: 48 - protein, 16 aa, synthetic construct
GGGGGGGSGGGGGGGS

SEQ ID NO: 49 - protein, 24 aa, synthetic construct
GGGGGGGSGGGGGGGSGGGGGGGS

SEQ ID NO: 50 - protein, 32 aa, synthetic construct
GGGGGGGSGGGGGGGSGGGGGGGSGGGGGGGS

SEQ ID NO: 51 - protein, 5 aa, synthetic construct
SGGGG

SEQ ID NO: 52 - protein, 10 aa, synthetic construct
SGGGGSGGGG

SEQ ID NO: 53 - protein, 15 aa, synthetic construct
SGGGGSGGGGSGGGG

SEQ ID NO: 54 - protein, 20 aa, synthetic construct
SGGGGSGGGGSGGGGSGGGG

SEQ ID NO: 55 - protein, 7 aa, synthetic construct
GGGGGGS

SEQ ID NO: 56 - protein, 14 aa, synthetic construct
GGGGGGSGGGGGGS

SEQ ID NO: 57 - protein, 21 aa, synthetic construct
GGGGGGSGGGGGGSGGGGGGS

SEQ ID NO: 58 - protein, 28 aa, synthetic construct
GGGGGGSGGGGGGSGGGGGGSGGGGGGS

SEQ ID NO: 59 - protein, 7 aa, synthetic construct
GGTFSHY

SEQ ID NO: 60 - protein, 6 aa, synthetic construct
IPEFGN

SEQ ID NO: 61 - protein, 10 aa, synthetic construct
GGTFSHYAIA

SEQ ID NO: 62 - protein, 10 aa, synthetic construct
GIIPEFGNAN

SEQ ID NO: 63 - protein, 8 aa, synthetic construct
GGTFSHYA

SEQ ID NO: 64 - protein, 8 aa, synthetic construct
IIPEFGNA

SEQ ID NO: 65 - protein, 16 aa, synthetic construct
AREGLYYRTSNYGMDV

SEQ ID NO: 66 - protein, 6 aa, synthetic construct
QSVSSN

SEQ ID NO: 67 - protein, 453 aa, synthetic construct
QVQLVQSGAEVKKPGSSVKVSCKASGGTFSHYAIAWVRQAPGQGLEWMGGIIPEFGNANYAQKFQGRVTITADESTSTAYMELSSLRSEDTAVYYCAREGLYYRTSNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, synthetic construct
EIVLTQSPATLSVSPGERATLSCRASQSVSSNLAWYQQKPGQAPRLLIYGASTRVTGIPARFSGSGSGTEFTLTISSLQSEDFAVYYCQQLSPWPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0139059 A1
(43) Pub. Date: May 21, 2026

(54) CD161 BINDING PROTEINS AND RELATED METHODS

(71) Applicant: Immunitas Therapeutics, Inc., Waltham, MA (US)

(72) Inventors: Matthew Alan HUGGINS, Acton, MA (US); Xianzhe WANG, Cambridge, MA (US); Frano-Joseph IRVINE, Medford, MA (US); Catherine Yue LIU, Hanover, NH (US); Elizabeth SCANLON, Allston, MA (US); Michael Benjamin BATTLES, Lebanon, NH (US)

(21) Appl. No.: 19/391,327

(22) Filed: Nov. 17, 2025

Related U.S. Application Data
(60) Provisional application No. 63/721,878, filed on Nov. 18, 2024.
(60) Provisional application No. 63/733,609, filed on Dec. 13, 2024.
(60) Provisional application No. 63/831,646, filed on Jun. 27, 2025.

(51) Int. Cl. C07K 16/28; A61K 39/00; A61P 17/06

(52) U.S. Cl. CPC C07K 16/2851; A61P 17/06; A61K 2039/505; C07K 2317/41; C07K 2317/732; C07K 2317/92

(57) ABSTRACT

Provided herein are CD161 binding proteins (e.g., antibodies) and compositions (e.g., pharmaceutical compositions) comprising the same; as well as methods of making the CD161 binding proteins (e.g., antibodies) and compositions. Further provided herein are kits comprising the CD161 binding proteins (e.g., antibodies) described herein. The CD161 binding proteins provided herein are useful in e.g., in therapeutic and diagnostic methods.

### RELATED APPLICATIONS

This application claims priority to U.S. Ser. No. 63/721,878, filed Nov. 18, 2024, U.S. Ser. No. 63/733,609, filed Dec. 13, 2024, and U.S. Ser. No. 63/831,646, filed Jun. 27, 2025, the entire contents of each of which is incorporated herein by reference.

### SEQUENCE LISTING

The instant application contains a Sequence Listing which has been submitted electronically in XML format and is hereby incorporated by reference in its entirety. Said XML copy, created on Nov. 15, 2025, is named 63340_32US01_SL.xml and is 65,870 bytes in size.

### 1. FIELD

This disclosure relates to CD161 binding proteins and nucleic acid molecules encoding the same. This disclosure is further related to methods of manufacturing and utilizing the same, including e.g., methods of treatment of a disease (e.g., a proinflammatory disease) in a subject in need thereof.

### 2. BACKGROUND

CD161 is a C-type lectin-like type-II transmembrane protein. CD161 is encoded by the killer cell lectin like receptor B1 (KLRB1) gene located within the natural killer (NK) cell gene complex on chromosome 12. CD161 is expressed by e.g., NK cells and subsets of both CD4+ and CD8+ T cells. CD161 binds CLEC2D, also a C-type lectin transmembrane protein. CLEC2D is expressed, e.g., on the surface of both malignant cells and immune cells including germinal center B cells, activated T cells, and tumor associated macrophages. CLEC2D/CD161 interactions play a role in regulating immune responses in various contexts.

### 3. SUMMARY

Provided herein are, inter alia, CD161 binding proteins and nucleic acid molecules encoding the same; fusions and conjugates comprising the CD161 binding proteins; methods of manufacturing; pharmaceutical compositions; and methods of use including e.g., methods of treating a disease (e.g., a proinflammatory disease) in a subject in need thereof, methods of inhibiting binding of CD161 to CLEC2D, and diagnostics.

Accordingly, in one aspect, provided herein are CD161 binding proteins comprising a binding domain that specifically binds CD161 (e.g., human CD161 (hCD161)), wherein the binding domain comprises: (a) a VH region comprising (i) a CDR-H1 comprising the amino acid sequence set forth in SEQ ID NO: 3 or the amino acid sequence set forth in SEQ ID NO: 3 comprising 1, 2, or 3 amino acid modifications; (ii) a CDR-H2 comprising the amino acid sequence set forth in SEQ ID NO: 4 or the amino acid sequence set forth in SEQ ID NO: 4 comprising 1, 2, or 3 amino acid modifications; and (iii) a CDR-H3 comprising the amino acid sequence set forth in SEQ ID NO: 5 or the amino acid sequence set forth in SEQ ID NO: 5 comprising 1, 2, or 3 amino acid modifications; and (b) a VL region comprising (i) a CDR-L1 comprising the amino acid sequence set forth in SEQ ID NO: 6 or the amino acid sequence set forth in SEQ ID NO: 6 comprising 1, 2, or 3 amino acid modifications; (ii) a CDR-L2 comprising the amino acid sequence set forth in SEQ ID NO: 7 or the amino acid sequence set forth in SEQ ID NO: 7 comprising 1, 2, or 3 amino acid modifications; and (iii) a CDR-L3 comprising the amino acid sequence set forth in SEQ ID NO: 8 or the amino acid sequence set forth in SEQ ID NO: 8 comprising 1, 2, or 3 amino acid modifications.

In one aspect, provided herein are CD161 binding protein comprising a binding domain that specifically binds CD161 (e.g., hCD161), wherein the binding domain comprises: (a) a VH region comprising (i) a CDR-H1 comprising the amino acid sequence set forth in SEQ ID NO: 3; (ii) a CDR-H2 comprising the amino acid sequence set forth in SEQ ID NO: 4; and (iii) a CDR-H3 comprising the amino acid sequence set forth in SEQ ID NO: 5; and (b) a VL region comprising (i) a CDR-L1 comprising the amino acid sequence set forth in SEQ ID NO: 6; (ii) a CDR-L2 comprising the amino acid sequence set forth in SEQ ID NO: 7; and (iii) a CDR-L3 comprising the amino acid sequence set forth in SEQ ID NO: 8.

In one aspect, provided herein are CD161 binding protein comprising a binding domain that specifically binds CD161 (e.g., hCD161), wherein the binding domain comprises: (a) a VH region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and (b) a VL region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10.

For the sake of clarity, it should be understood that the following embodiments are applicable to each of the foregoing aspects as if recited directly following each individual aspect.

In some embodiments, (a) the amino acid sequence of the VH region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and (b) the amino acid sequence of the VL region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10.

In some embodiments, (a) the amino acid sequence of the VH region is at least 90%, 91%, 92%, 93%, 94%, 95%, 96%, 97%, 98%, 99%, or 100% identical to the amino acid sequence set forth in SEQ ID NO: 9; and (b) the amino acid sequence of the VL region is at least 90%, 91%, 92%, 93%, 94%, 95%, 96%, 97%, 98%, 99%, or 100% identical to the amino acid sequence set forth in SEQ ID NO: 10.

In some embodiments, (a) the amino acid sequence of the VH region is at least 95%, 96%, 97%, 98%, 99%, or 100% identical to the amino acid sequence set forth in SEQ ID NO: 9; and (b) the amino acid sequence of the VL region is at least 95%, 96%, 97%, 98%, 99%, or 100% identical to the amino acid sequence set forth in SEQ ID NO: 10.

In some embodiments, (a) the amino acid sequence of the VH region is at least 99% identical to the amino acid sequence set forth in SEQ ID NO: 9; and (b) the amino acid sequence of the VL region is at least 99% identical to the amino acid sequence set forth in SEQ ID NO: 10.

In some embodiments, (a) the amino acid sequence of the VH region comprises the amino acid sequence set forth in SEQ ID NO: 9; and (b) the amino acid sequence of the VL region comprises the amino acid sequence set forth in SEQ ID NO: 10.

In some embodiments, the CD161 binding protein comprises a heavy chain (HC). In some embodiments, the CD161 binding protein comprises a light chain (LC).

In some embodiments, the CD161 binding protein comprises a heavy chain (HC) and a light chain (LC).

In some embodiments, the CD161 binding protein comprises the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68.

In some embodiments, the CD161 binding protein (or the binding domain) comprises an antibody.

In some embodiments, the CD161 binding protein (or the binding domain) comprises an IgG (e.g., IgG₁, IgG₂ (e.g., IgG₂ₐ or IgG_{2b}), IgG₃, IgG₄), IgE, IgM, IgD, or IgA (e.g., IgA₁ or IgA₂) antibody.

In some embodiments, the CD161 binding protein (or the binding domain) comprises one or more of a monoclonal antibody, monospecific antibody, multispecific antibody, human antibody, humanized antibody, chimeric antibody, and/or murine antibody, or a functional fragment or functional variant of any of the foregoing.

In some embodiments, the CD161 binding protein (or the binding domain) comprises one or more of a full-length antibody, scFv, Fab, F(ab′)₂, Fab′, Fv, single domain antibody (e.g., a VHH), scFv-Fc, Fab-Fc, and/or single domain antibody-Fc (e.g., VHH-Fc).

In some embodiments, the CD161 binding protein (e.g., an anti-CD161 antibody) comprises a hIg Fc region.

In some embodiments, the hIg Fc region is capable of mediating one or more hIg Fc effector function. In some embodiments, the hIg Fc region is capable of mediating one or more of ADCC, ADCP, or CDC. In some embodiments, the hIg Fc region is capable of mediating ADCC.

In some embodiments, the hIg Fc region comprises one or more amino acid variation or alteration (e.g., described herein (e.g., Table 4 herein)) in glycosylation that enhances one or more hIg Fc effector function (e.g., compared to a reference hIg Fc region or a reference CD161 binding protein comprising the same).

In some embodiments, the hIg Fc region comprises one or more amino acid variation or alteration in glycosylation that enhances ADCC and/or ADCP (e.g., compared to a reference hIg Fc region or a reference CD161 binding protein comprising the same).

In some embodiments, the hIg Fc region is afucosylated.

In some embodiments, the CD161 binding protein (e.g., an anti-CD161 antibody) (e.g., described herein) is capable of inhibiting (blocking) the binding of CD161 to CLEC2D.

In some embodiments, the CD161 binding protein (e.g., the anti-CD161 antibody) is capable of mediating depletion of a population of CD161 expressing cells (e.g., immune cells (e.g., T cells (e.g., activated T cells) (e.g., CD4+ T cells, CD8+ T cells), NK cells, B cells, MAIT cells, gd T cells, Tregs, and/or ILCs)) (e.g., upon binding to CD161 expressed on the surface of the population of cells).

In some embodiments, the depletion of the population of CD161 expressing cells (e.g., immune cells (e.g., T cells (e.g., activated T cells) (e.g., CD4+ T cells, CD8+ T cells), NK cells, MAIT cells, gd T cells, Tregs, Th2A cells, and/or ILCs)) mediates a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more pro-inflammatory cytokine is IL-4, IL-13, IL-5 or IL-9. In some embodiments, the population of CD161 expressing cells comprises Th2A cells and the one or more pro-inflammatory cytokine is IL-4, IL-13, IL-5 or IL-9.

In some embodiments, the CD161 binding protein (e.g., the anti-CD161 antibody) is capable of mediating a reduction in the level of one or more proinflammatory cytokine (e.g., IFNγ, TNFα, IL-17, IL-22, IL-21, or GM-CSF) (e.g., produced by a population of cells (e.g., immune cells (e.g., T cells (e.g., activated T cells) (e.g., CD4+ T cells, CD8+ T cells), NK cells, MAIT cells, gd T cells, Tregs, and/or ILCs))). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the CD161 binding protein (e.g., an anti-CD161 antibody) mediates depletion of the population of cells through one or more hIg Fc effector function.

In some embodiments, the CD161 binding protein (e.g., an anti-CD161 antibody) mediates depletion of the population of cells through any one or more of antibody dependent cellular cytotoxicity (ADCC), antibody dependent cellular phagocytosis (ADCP), or complement dependent cytotoxicity (CDC).

In some embodiments, the CD161 binding protein comprises a heterologous moiety (e.g., a heterologous protein). In some embodiments, the CD161 binding protein comprises 2, 3, 4, or 5 or more heterologous moieties.

In some embodiments, the heterologous moiety is attached to the N-terminus, C-terminus, and/or internally between the N- and C-terminus of the CD161 binding protein. In some embodiments, the heterologous moiety (e.g., heterologous polypeptide) is directly attached to the CD161 binding protein. In some embodiments, the heterologous moiety (e.g., heterologous polypeptide) is indirectly attached to the CD161 binding protein. In some embodiments, the heterologous moiety (e.g., heterologous polypeptide) is indirectly attached to the CD161 binding protein via a linker.

In some embodiments, the heterologous moiety is a peptide, protein (e.g., antibody), nucleic acid molecule (e.g., DNA, RNA), carbohydrate, lipid, polymer, or small molecule. In some embodiments, the heterologous moiety is a half-life extension moiety (e.g., half-life extension protein (e.g., Fc region)). In some embodiments, the heterologous moiety is a detectable tag and/or a reporter gene. In some embodiments, the heterologous moiety is a therapeutic agent.

In some embodiments, the CD161 binding protein is isolated. In some embodiments, the CD161 binding protein is recombinant.

In one aspect, provided herein are conjugated comprising a CD161 binding protein described herein and a heterologous moiety. In some embodiments, the conjugate comprises 2, 3, 4, or 5 or more heterologous moieties.

In some embodiments, the heterologous moiety is attached to the N-terminus, C-terminus, and/or internally between the N- and C-terminus of the CD161 binding protein. In some embodiments, the heterologous moiety (e.g., heterologous polypeptide) is directly attached to the CD161 binding protein. In some embodiments, the heterologous moiety (e.g., heterologous polypeptide) is indirectly attached to the CD161 binding protein. In some embodiments, the heterologous moiety (e.g., heterologous polypeptide) is indirectly attached to the CD161 binding protein via a linker.

In some embodiments, the heterologous moiety is a peptide, protein, carbohydrate, lipid, polymer, or small molecule. In some embodiments, the heterologous moiety is a therapeutic agent. In some embodiments, the therapeutic agent is a small molecule.

In one aspect, provided herein are fusion proteins comprising a CD161 binding protein described herein and a heterologous protein.

In some embodiments, the heterologous protein comprises a cytokine (or functional fragment or functional variant thereof), a chemokine (or a functional fragment or functional variant thereof), or an antibody (or a functional fragment or functional variant thereof).

In some embodiments, the heterologous protein is fused to the N-terminus, C-terminus, and/or internally between the N- and C-terminus of the CD161 binding protein. In some embodiments, the heterologous protein is fused directly to the CD161 binding protein. In some embodiments, the heterologous protein is fused indirectly to the CD161 binding protein. In some embodiments, the heterologous protein is fused indirectly to the CD161 binding protein via a peptide linker.

In one aspect, provided herein are nucleic acid molecule encoding a CD161 binding protein described herein or a fusion protein described herein.

In some embodiments, the nucleic acid molecule is a DNA or RNA (e.g., mRNA) molecule. In some embodiments, the nucleic acid molecule is codon optimized. In some embodiments, the nucleic acid molecule comprises one or more transcription or translation regulatory elements (e.g., promoter, enhancer (e.g., cell or tissue specific transcription regulatory elements).

In one aspect, provided herein are vectors comprising a nucleic acid molecule described herein. In some embodiments, the vector is a viral vector or a non-viral vector (e.g., plasmid, minicircle). In some embodiments, the carrier is a viral vector (e.g., an adeno associated viral (AAV) vector, a lentiviral vector, an adenoviral vector).

In one aspect, provided herein are carriers comprising a CD161 binding protein described herein, a fusion protein described herein, a conjugate described herein, a nucleic acid molecule described herein, a vector described herein, a cell (or population of cells) described herein, or a pharmaceutical composition described herein. In some embodiments, the carrier is a nanoparticle, polymer, virus (e.g., a recombinant virus), virus like particle, virosome, fusosome, vesicle, or lipid-based carrier (e.g., a lipid nanoparticle (LNP), liposome, lipoplex, nanoliposome, an exosome, or a micelle).

In one aspect, provided herein are cells or population of cells comprising a CD161 binding protein described herein, a fusion protein described herein, a conjugate described herein, a nucleic acid molecule described herein, a vector described herein, a carrier described herein, or a pharmaceutical composition described herein.

In one aspect, provided herein are pharmaceutical compositions comprising a CD161 binding protein described herein, a fusion protein described herein, a conjugate described herein, a nucleic acid molecule described herein, a vector described herein, a carrier described herein, or a cell or population of cells described herein, and a pharmaceutically acceptable excipient.

In one aspect, provided herein are kits comprising a CD161 binding protein described herein, a fusion protein described herein, a conjugate described herein, a nucleic acid molecule described herein, a vector described herein, a carrier described herein, a cell or population of cells described herein, or a pharmaceutical composition described herein; and optionally instructions for using any one or more of the foregoing.

In one aspect, provided herein are methods of manufacturing a CD161 binding protein described herein, a fusion protein described herein, a conjugate described herein, the method comprising: introducing into a cell a nucleic acid molecule described herein, a vector described herein, or a carrier described herein; culturing the cell under conditions that allow for expression of the CD161 binding protein, the conjugate, or the fusion protein; and optionally recovering the expressed the CD161 binding protein, the conjugate, or the fusion protein from the culture; and optionally purifying the expressed the CD161 binding protein, the conjugate, or the fusion protein from the culture.

In one aspect, provided herein are methods of manufacturing an afucosylated CD161 binding protein described herein, a fusion protein described herein, a conjugate described herein, the method comprising: introducing into a cell that comprises a disruption in the FUT8 gene a nucleic acid molecule described herein, a vector described herein, or a carrier described herein; culturing the cell under conditions that allow for expression of the afucosylated CD161 binding protein, the conjugate, or the fusion protein; and optionally recovering the expressed the CD161 binding protein, the conjugate, or the fusion protein from the culture; and optionally purifying the expressed the CD161 binding protein, the conjugate, or the fusion protein from the culture.

In one aspect, provided herein are methods of delivering a CD161 binding protein described herein, a conjugate described herein, a fusion protein described herein, a nucleic acid molecule described herein, a vector described herein, a carrier described herein, a cell (or population of cells) described herein, or a pharmaceutical composition described herein to a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby deliver the CD161 binding protein, conjugate, fusion protein, nucleic acid molecule, vector, carrier, cell (or population of cells), or pharmaceutical composition to the subject.

In one aspect, provided herein are methods of treating a proinflammatory disease (e.g., an autoimmune disease) in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby treat the proinflammatory disease (e.g., the autoimmune disease) in the subject.

In some embodiments, the proinflammatory disease (e.g., the autoimmune disease) is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis. In some embodiments, the proinflammatory disease is an autoimmune disease, an idiopathic inflammatory myopathies (e.g., dermatomyositis, polymyositis, anti synthetase syndrome, inclusion-body myositis), sarcoidosis, enteropathic arthritis, or palmoplantar pustulosis.

In some embodiments, the proinflammatory disease is an allergic disease (e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (e.g., eosinophilic esophagitis, hypereosinophilic syndrome, allergic rhinoconjunctivitis, an IgE mediated disease (e.g., bullous pemphigoid, lupus nephritis, pemphigus, autoimmune pancreatitis, and chronic spontaneous urticaria).

In some embodiments, the autoimmune disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis, an idiopathic inflammatory myopathies (e.g., dermatomyositis, polymyositis, anti synthetase syndrome, inclusion-body myositis), or sarcoidosis, enteropathic arthritis, or palmoplantar pustulosis.

In some embodiments, the autoimmune disease is an allergic disease (e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (e.g., eosinophilic esophagitis, hypereosinophilic syndrome, allergic rhinoconjunctivitis, an IgE mediated disease (e.g., bullous pemphigoid, lupus nephritis, pemphigus, autoimmune pancreatitis, and chronic spontaneous urticaria).

In some embodiments, the proinflammatory disease (e.g., an autoimmune disease) is treated, at least in part, through the depletion of a population of CD161 expressing cells (e.g., immune cells (e.g., T cells (e.g., CD4+ T cells, CD8+ T cells, Th2 cells, peTh2 cells, TH2A cells, ILC2 cells), NK cells, MAIT cells, gd T cells, Tregs, and/or ILCs)) in the subject.

In some embodiments, the CD161 binding protein (e.g., the anti-CD161 antibody) mediates depletion of a population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, B cells, MAIT cells, gd T cells, Tregs, and/or ILCs)) in the subject.

In some embodiments, the depletion of the population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs)) mediates a reduction in the level of one or more proinflammatory cytokine (e.g., IFNγ, TNFα, IL-17, IL-22, IL-21, GM-CSF, IL-4, IL-13, IL-5 or IL-9). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the depletion of the population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs)) mediates a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the proinflammatory disease (e.g., an autoimmune disease) is treated, at least in part, through a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the CD161 binding protein (e.g., the anti-CD161 antibody) mediates a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the reduction in the level of one or more proinflammatory cytokine (e.g., IFNγ, TNFα, IL-17, IL-22, IL-21, or GM-CSF) is mediated by the depletion of the population of CD161 expressing cells (e.g., immune cells (e.g., T cells (e.g., CD4+ T cells, CD8+ T cells, Th2 cells, peTh2 cells, TH2A cells, ILC2 cells), NK cells, MAIT cells, gd T cells, Tregs, and/or ILCs)). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the population of CD161 expressing cells comprises one or more subpopulations of immune cells.

In some embodiments, the population of CD161 expressing cells comprises T cells (e.g., activated T cells) (e.g., CD4+ T cells and/or CD8+ T cells).

In some embodiments, the population of CD161 expressing cells comprises CD4+ T cells and/or CD8+ T cells (e.g., activated CD4+ T cells and/or CD8+ T cells).

In some embodiments, the population of CD161 expressing cells comprises Th2 cells, peTh2 cells, TH2A cells, and/or ILC2 cells.

In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In one aspect, provided herein are methods of treating an autoimmune disease in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby treat the autoimmune disease in the subject.

In some embodiments, the autoimmune disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis, an idiopathic inflammatory myopathies (e.g., dermatomyositis, polymyositis, anti synthetase syndrome, inclusion-body myositis), or sarcoidosis, enteropathic arthritis, or palmoplantar pustulosis.

In some embodiments, the autoimmune disease is an allergic disease (e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (e.g., eosinophilic esophagitis, hypereosinophilic syndrome, allergic rhinoconjunctivitis, an IgE mediated disease (e.g., bullous pemphigoid, lupus nephritis, pemphigus, autoimmune pancreatitis, and chronic spontaneous urticaria).

In some embodiments, the autoimmune disease is treated, at least in part, through the depletion of a population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, B cells, MAIT cells, gd T cells, Tregs, and/or ILCs)) in the subject.

In some embodiments, the CD161 binding protein (e.g., the anti-CD161 antibody) mediates depletion of a population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs)) in the subject.

In some embodiments, the depletion of the population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs)) mediates a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the autoimmune disease is treated, at least in part, through a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the CD161 binding protein (e.g., the anti-CD161 antibody) mediates a reduction in the level of one or more proinflammatory cytokine (e.g., IFNγ, TNFα, IL-17, IL-22, IL-21, or GM-CSF). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the reduction in the level of one or more proinflammatory cytokine (e.g., IFNγ, TNFα, IL-17, IL-22, IL-21, or GM-CSF) is mediated by the depletion of the population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs)). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the population of CD161 expressing cells comprises one or more subpopulations of immune cells.

In some embodiments, the population of CD161 expressing cells comprises T cells (e.g., activated T cells) (e.g., CD4+ T cells and/or CD8+ T cells).

In some embodiments, the population of CD161 expressing cells comprises CD4+ T cells and/or CD8+ T cells (e.g., activated CD4+ T cells and/or CD8+ T cells).

In some embodiments, the population of CD161 expressing cells comprises Th2 cells, peTh2 cells, TH2A cells, and/or ILC2 cells.

In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In one aspect, provided herein are methods of depleting a population of cells (e.g., immune cells (e.g., T cells, NK cells, MAIT cells, gd T cells, Tregs, and/or ILCs)) expressing CD161 (e.g., on the surface) in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby deplete the population of cells (e.g., immune cells (e.g., T cells, NK cells, MAIT cells, gd T cells, Tregs, and/or ILCs)) expressing CD161 (e.g., on the surface) in the subject.

In some embodiments, the population of cells comprises T cells (e.g., activated T cells) (e.g., CD4+ T cells, CD8+ T cells, Th2 cells, peTh2 cells, TH2A cells, ILC2 cells), NK cells, MAIT cells, gd T cells, Tregs, and/or ILCs.

In some embodiments, the population of cells comprises T cells (e.g., activated T cells) (e.g., CD4+ T cells, CD8+ T cells, Th2 cells, peTh2 cells, TH2A cells, ILC2 cells).

In some embodiments, the population of cells comprises CD4+ T cells and CD8+ T cells (e.g., activated CD4+ T cells and CD8+ T cells).

In some embodiments, the population of cells expresses one or more proinflammatory cytokine.

In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the CD161 binding protein (e.g., an anti-CD161 antibody) comprises a human Ig (hIg) Fc region.

In some embodiments, the CD161 binding protein (e.g., an anti-CD161 antibody) mediates depletion of the population of cells through one or more hIg Fc effector function.

In some embodiments, the CD161 binding protein (e.g., an anti-CD161 antibody) mediates depletion of the population of cells through any one or more of antibody dependent cellular cytotoxicity (ADCC), antibody dependent cellular phagocytosis (ADCP), or complement dependent cytotoxicity (CDC).

In some embodiments, the CD161 binding protein (e.g., an anti-CD161 antibody) mediates depletion of the population of cells, at least in part, through ADCC.

In one aspect, provided herein are methods of reducing the level of one or more proinflammatory cytokines in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby reduce the level of one or more proinflammatory cytokines in the subject.

In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 and IL-9.

In some embodiments, the level of a plurality of proinflammatory cytokines is reduced (e.g., simultaneously).

In some embodiments, the level of at least 2, 3, 4, 5, or 6, or more proinflammatory cytokines is reduced (e.g., simultaneously).

In some embodiments, the CD161 binding protein (e.g., an anti-CD161 antibody) comprises a human Ig (hIg) Fc region.

In some embodiments, the reduction in the level of one or more proinflammatory cytokine (e.g., IFNγ, TNFα, IL-17, IL-22, IL-21, or GM-CSF) is mediated by the depletion of a population of CD161 expressing cells (e.g., immune cells (e.g., T cells (e.g., CD4+ T cells, CD8+ T cells, Th2 cells, peTh2 cells, TH2A cells, ILC2 cells), NK cells, MAIT cells, gd T cells, Tregs, and/or ILCs)). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 and IL-9.

In some embodiments, the population of CD161 expressing cells comprises one or more subpopulations of immune cells.

In some embodiments, the population of CD161 expressing cells comprises T cells (e.g., activated T cells) (e.g., CD4+ T cells and/or CD8+ T cells).

In some embodiments, the population of CD161 expressing cells comprises CD4+ T cells and/or CD8+ T cells (e.g., activated CD4+ T cells and/or CD8+ T cells).

In some embodiments, the population of CD161 expressing cells comprises Th2 cells, peTh2 cells, TH2A cells, and/or ILC2 cells.

In some embodiments, the CD161 binding protein (e.g., an anti-CD161 antibody) mediates depletion of the population of cells through one or more hIg Fc effector function.

In some embodiments, the CD161 binding protein (e.g., an anti-CD161 antibody) mediates depletion of the population of cells through any one or more of ADCC, ADCP, or CDC.

In some embodiments, the CD161 binding protein (e.g., an anti-CD161 antibody) mediates depletion of the population of cells, at least in part, through ADCC.

In one aspect, provided herein are methods of inhibiting (blocking) binding of CD161 to CLEC2D expressed on the surface of a cell in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby inhibit (blocking) binding of CD161 to CLEC2D expressed on the surface of a cell in the subject.

In one aspect, provided herein are methods of treating cancer in a subject in need thereof, the method comprising administering to the subject a CD161 binding protein described herein, a fusion protein described herein, a conjugate described herein, a nucleic acid molecule described herein, a vector described herein, a carrier described herein, a cell or population of cells described herein, or a pharmaceutical composition described herein, to thereby treat the cancer in the subject.

In some embodiments, the cancer is a leukemia or lymphoma. In some embodiments, the cancer is a T cell leukemia, NK cell leukemia, T cell prolymphocytic leukemia (T-PLL), or large granular lymphocytic leukemia (LGLL). In some embodiments, the cancer is a NK/T cell lymphoma (NKTCL), extranodal NK/T cell lymphoma (ENKL), mycosis fungoides (MF), Sezary syndrome, peripheral T cell lymphoma, angioimmunoblastic T cell lymphoma (AITL), and peripheral T cell lymphoma not otherwise specified (PTCL-NOS).

In some embodiments, a CD161 binding protein described herein or a fusion protein described herein is administered to the subject.

In some embodiments, the CD161 binding protein, conjugate, fusion protein, nucleic acid molecule, vector, carrier, cell (or population of cells), or a pharmaceutical composition is administered in combination with one or more additional therapeutic agent. In some embodiments, the CD161 binding protein, conjugate, fusion protein, nucleic acid molecule, vector, carrier, cell (or population of cells), or a pharmaceutical composition is administered prior to, concomitant with, and/or after the administration of one or more additional therapeutic agent. In some embodiments, the one or more additional therapeutic agent is an anti-inflammatory agent. In some embodiments, the subject is human.

### 4. BRIEF DESCRIPTION OF THE DRAWINGS

FIG. 1 is a line graph showing the binding of Ab-1 (SEQ ID NOS: 67-68) (at the indicated concentration) to CD161 expressed on the surface of HEK cells (as measured by flow cytometry). Binding expressed as mean fluorescence intensity.

FIG. 2 is a line graph showing the binding of Ab-1 (SEQ ID NOS: 67-68) (at the indicated concentration) to human CD161 expressed on the surface of CD4+ T cells.

FIG. 3 is a line graph showing the binding of Ab-1 (SEQ ID NOS: 67-68) (at the indicated concentration) to cyno CD161 expressed on the surface of CD4+ T cells.

FIG. 4 is a line graph showing the binding of CD161 expressed on the surface of cells in vitro to biotinylated CLEC2D-Fc fusion in presence of Ab-1 (at the indicated concentration) (or no antibody control).

FIG. 5 is a line graph showing Ab-1 induced ADCC mediated killing of CD161 expressing Jurkat cells (at the indicated concentration of Ab-1).

FIG. 6 is a line graph showing Ab-1 induced ADCC mediated killing of CD161 expressing human T cells (at the indicated concentration of Ab-1).

FIG. 7 is a line graph showing Ab-1 and afucosylated Ab-1 induced ADCC mediated killing of CD161 expressing CD4+ T cells (at the indicated concentration of Ab-1).

FIG. 8A is a line graph showing the total PASI (Psoriasis Area and Severity index) score of cynomolgus monkeys treated with 10 mg/kg afucosylated Ab-1 (or vehicle control) in the IMQ mediated Psoriasis model. FIG. 8B is a line graph showing the total PASI (Psoriasis Area and Severity index) score of cynomolgus monkeys treated with 20 mg/kg afucosylated Ab-1 (or vehicle control) in the IMQ mediated Psoriasis model.

FIG. 9 is a line graph showing the total PASI (Psoriasis Area and Severity index) score of cynomolgus monkeys treated with afucosylated Ab-1 (10 mg/kg) or a reference anti-IL-17 antibody (3 mg/kg) in the IMQ mediated Psoriasis model.

FIG. 10A is a line graph showing the erythema score of cynomolgus monkeys treated with afucosylated Ab-1 (10 mg/kg or 20 mg/kg), a reference anti-IL-17 antibody (3 mg/kg), or a vehicle control in the IMQ mediated Psoriasis model. FIG. 10B is a line graph showing the scaling score of cynomolgus monkeys treated with afucosylated Ab-1 (10 mg/kg or 20 mg/kg), a reference anti-IL-17 antibody (3 mg/kg), or a vehicle control in the IMQ mediated Psoriasis model. FIG. 10C is a line graph showing the thickening score of cynomolgus monkeys treated with afucosylated Ab-1 (10 mg/kg or 20 mg/kg), a reference anti-IL-17 antibody (3 mg/kg), or a vehicle control in the IMQ mediated Psoriasis model. FIG. 10D is a line graph showing the total PASI score of cynomolgus monkeys treated with afucosylated Ab-1 (10 mg/kg or 20 mg/kg), a reference anti-IL-17 antibody (3 mg/kg), or a vehicle control in the IMQ mediated Psoriasis model.

FIG. 11 shows photographic images of the skin inflammation of cynomolgus monkeys treated with afucosylated Ab-1 (10 mg/kg or 20 mg/kg), a reference anti-IL-17 antibody (3 mg/kg), or a vehicle control in the IMQ mediated Psoriasis model. Representative images at Day 14.

FIG. 12 is a line graph showing the total PASI (Psoriasis Area and Severity index) score of cynomolgus monkeys treated with afucosylated Ab-1 (10 mg/kg or 20 mg/kg) or a reference anti-IL-17 antibody (3 mg/kg) in the IMQ mediated Psoriasis model through day 29 of treatment.

FIG. 13A is a line graph showing the total PASI score for individual cynomolgus monkeys in the IMQ mediated Psoriasis model treated with vehicle control through day 29. FIG. 13B is a line graph showing the total PASI score for individual cynomolgus monkeys in the IMQ mediated Psoriasis model treated with a reference anti-IL-17 antibody (3 mg/kg) through day 29. FIG. 13C is a line graph showing the total PASI score for individual cynomolgus monkeys in the IMQ mediated Psoriasis model treated with afucosylated Ab-1 (10 mg/kg) through day 29. FIG. 13D is a line graph showing the total PASI score for individual cynomolgus monkeys in the IMQ mediated Psoriasis model treated with afucosylated Ab-1 (20 mg/kg) through day 29.

FIG. 14A is a line graph showing the average skin erythema of cynomolgus monkeys in the IMQ mediated Psoriasis model treated with afucosylated Ab-1 (10 mg/kg or 20 mg/kg), a reference anti-IL-17 antibody (3 mg/kg), or a vehicle control through day 29. FIG. 14B is a line graph showing the average skin thickening of cynomolgus monkeys in the IMQ mediated Psoriasis model treated with afucosylated Ab-1 (10 mg/kg or 20 mg/kg), a reference anti-IL-17 antibody (3 mg/kg), or a vehicle control through day 29. FIG. 14C is a line graph showing the average skin scaling of cynomolgus monkeys in the IMQ mediated Psoriasis model treated with afucosylated Ab-1 (10 mg/kg or 20 mg/kg), a reference anti-IL-17 antibody (3 mg/kg), or a vehicle control through day 29. FIG. 14D is a line graph showing the average total PASI score of cynomolgus monkeys in the IMQ mediated Psoriasis model treated with afucosylated Ab-1 (10 mg/kg or 20 mg/kg), a reference anti-IL-17 antibody (3 mg/kg), or a vehicle control through day 29.

FIG. 15A is a bar graph showing the average total PASI score of cynomolgus monkeys in the IMQ mediated Psoriasis model treated with afucosylated Ab-1 (10 mg/kg or 20 mg/kg), a reference anti-IL-17 antibody (3 mg/kg), or a vehicle control as area under the curve (AUC) measurements. FIG. 15B is a bar graph showing the average skin thickening of cynomolgus monkeys in the IMQ mediated Psoriasis model treated with afucosylated Ab-1 (10 mg/kg or 20 mg/kg), a reference anti-IL-17 antibody (3 mg/kg), or a vehicle control as area under the curve (AUC) measurements. FIG. 15C is a bar graph showing the average skin scaling of cynomolgus monkeys in the IMQ mediated Psoriasis model treated with afucosylated Ab-1 (10 mg/kg or 20 mg/kg), a reference anti-IL-17 antibody (3 mg/kg), or a vehicle control as area under the curve (AUC) measurements. FIG. 15D is a bar graph showing the average skin erythema of cynomolgus monkeys in the IMQ mediated Psoriasis model treated with afucosylated Ab-1 (10 mg/kg or 20 mg/kg), a reference anti-IL-17 antibody (3 mg/kg), or a vehicle control as area under the curve (AUC) measurements.

FIG. 16A is a FACS plot showing the number of CD161 expressing T cells from the peripheral blood of cynomolgus monkeys in the IMQ mediated Psoriasis model treated with afucosylated Ab-1 (20 mg/kg) or a vehicle control. FIG. 16B is a line graph showing the percent of CD161 expressing T cells from the peripheral blood of cynomolgus monkeys in the IMQ mediated Psoriasis model treated with afucosylated Ab-1 (20 mg/kg) (expressed as percent of total CD3+ cells) over 14 days.

FIG. 17A is dot plot showing the frequency of CD161 expressing CD8+ T cells in intestinal biopsies of cynomolgus monkeys in the IMQ mediated Psoriasis model treated with afucosylated Ab-1 (20 mg/kg) or vehicle control at day 17. FIG. 17B is dot plot showing the frequency of CD161 expressing CD4+ T cells in intestinal biopsies of cynomolgus monkeys in the IMQ mediated Psoriasis model treated with afucosylated Ab-1 (20 mg/kg) or vehicle control at day 17. FIG. 17C is dot plot showing the frequency of CD161 expressing NK cells in intestinal biopsies of cynomolgus monkeys in the IMQ mediated Psoriasis model treated with afucosylated Ab-1 (20 mg/kg) or vehicle control at day 17.

FIG. 18A is a bar graph showing the average histopathology score of skin biopsies from cynomolgus monkeys in the IMQ mediated Psoriasis model treated with afucosylated Ab-1 (10 mg/kg or 20 mg/kg), a reference anti-IL-17 antibody (3 mg/kg), or a vehicle control at day 0, day 7, or day 14. FIG. 18B is a bar graph showing the average lymphocyte infiltrate score of skin biopsies from cynomolgus monkeys in the IMQ mediated Psoriasis model treated with afucosylated Ab-1 (10 mg/kg or 20 mg/kg), a reference anti-IL-17 antibody (3 mg/kg), or a vehicle control at day 0, day 7, or day 14.

FIG. 19 presents microscopy images of hematoxylin and eosin stained skin biopsies from cynomolgus monkeys in the IMQ mediated Psoriasis model treated with afucosylated Ab-1 (10 mg/kg) or a vehicle control at day 0 or day 14.

FIG. 20A is a heat map showing the change in expression level of the indicated pro-inflammatory cytokines in skin biopsies from cynomolgus monkeys in the IMQ mediated Psoriasis model treated with afucosylated Ab-1 (10 mg/kg) or vehicle control at day 15. FIG. 20B is a heat map showing the change in expression level of the indicated chemokines in skin biopsies from cynomolgus monkeys in the IMQ mediated Psoriasis model treated with afucosylated Ab-1 (10 mg/kg) or vehicle control at day 15. FIG. 20C is a heat map showing the change in expression level of the indicated psoriatic skin lesion associated genes in skin biopsies from cynomolgus monkeys in the IMQ mediated Psoriasis model treated with afucosylated Ab-1 (10 mg/kg) or vehicle control at day 15.

FIG. 21 is a line graph showing afucosylated Ab-1 induced ADCC mediated killing of CD161 expressing cyno CD8+ T cells (at the indicated concentration of Ab-1).

FIG. 22A is a table showing Gene Set Enrichment Analysis (GSEA) of bulk RNA-sequencing data from Day 15 skin biopsies in the NHP imiquimod (IMQ) model comparing afucosylated Ab-1 (10 mg/kg)-treated animals to vehicle controls. FIG. 22B is a series of graphs showing Gene Set Variation Analysis (GSVA) quantification of pathway-specific enrichment scores for each individual sample (afucosylated Ab-1 treatment or vehicle control). “Ab-1” in FIG. 22B refers to afucosylated Ab-1.

FIG. 23 is a schematic showing the design of the IMQ study.

FIG. 24 is a FACS plot showing the number of CD161+CD3+ T cells from skin biopsies taken at day 21 from the indicated treatment group (vehicle control; 3× dose of 10 mg/kg afucosylated Ab-1 treatment group; and single dose of 30 mg/kg afucosylated Ab-1 treatment group).

FIG. 25 is a bar graph showing the absolute counts of CD161+ T cells in the skin at day 21 from the indicated treatment group (vehicle control; 3× dose of 10 mg/kg afucosylated Ab-1 treatment group; and single dose of 30 mg/kg afucosylated Ab-1 treatment group).

FIG. 26 is a bar graph showing the percent reduction in CD161+ T cells in the skin at day 21 from the indicated treatment group (vehicle control; 3× dose of 10 mg/kg afucosylated Ab-1 treatment group; and single dose of 30 mg/kg afucosylated Ab-1 treatment group).

FIG. 27 is a series of line graphs showing the PASI score over 1-21 days in each of the indicated treatment groups (vehicle control; 3× dose of 10 mg/kg treatment group; and single dose of 30 mg/kg treatment group). “Ab-1” in FIG. 27 refers to afucosylated Ab-1.

FIG. 28 is a heat map showing the change in expression level of the indicated gene (cytokine, chemokine, or psoriasis skin lesion associated gene) in skin biopsies from cynomolgus monkeys in the IMQ mediated Psoriasis model from the indicated treatment group (vehicle control; 3× dose of 10 mg/kg treatment group; and single dose of 30 mg/kg treatment group) at day 21.

FIG. 29A is a table showing Gene Set Enrichment Analysis (GSEA) of bulk RNA-sequencing data from Day 21 skin biopsies in the NHP imiquimod (IMQ) model comparing Ab-1-treated animals to vehicle controls. FIG. 29B is a series of graphs showing Gene Set Variation Analysis (GSVA) quantification of pathway-specific enrichment scores for each individual sample (Afucosylated Ab-1 treatment or vehicle control). “Ab-1” in FIG. 29B refers to afucosylated Ab-1.

FIG. 30A is a dot graph showing the number of neutrophils in skin sample at day 21 of animals treated with vehicle control or afucosylated Ab-1; along with bar graphs showing the level of CXCL1 and CXCL8 at day 7 and day 21 in the indicated treatment group (vehicle control or afucosylated Ab-1). FIG. 30B is a dot graph showing the number of macrophages in skin sample at day 21 of animals treated with vehicle control or afucosylated Ab-1; along with bar graph showing the level of CCL2/MCP1 and CXCL12 at day 7 and day 21 in the indicated treatment group (vehicle control or afucosylated Ab-1).

FIG. 31 is a heat map showing Gene Set Enrichment Analysis (GSEA) of bulk RNA-sequencing data from Day 21 skin biopsies in the NHP IMQ model comparing vehicle control, afucosylated Ab-1-treatment group, anti-IL-23 antibody treatment, and anti-TNF antibody treatment group. “Ab-1” in FIG. 31 refers to afucosylated Ab-1.

FIG. 32 is a series of graphs showing Gene Set Variation Analysis (GSVA) quantification of pathway-specific enrichment scores for each individual sample (vehicle control, afucosylated Ab-1-treatment group, anti-IL-23 antibody treatment, and anti-TNF antibody treatment group).

FIG. 33A is a plot and a bar graph showing the percentage of CD4+CD161+ T cells and CD8+CD161+ T cells in samples of intestinal tissue from Crohn's disease patients. FIG. 33B is a series of bar graphs showing the level of IL-17F, IL-22, IFNγ, and TNFα expressed by CD161-cells and CD161+ cells in samples of intestinal tissue from Crohn's disease patients.

FIG. 34 is a series of line graphs showing the concentration (pg/mL) of each of the indicated cytokines or chemokines (Th 17 cytokines—IL17f, IL-22; Pro-inflammatory cytokines—GMCSF, IFNγ, TNFα; and IL 17 regulated chemokines—CXCL5, CCL20, and IL8) from samples of intestinal tissue from Crohn's disease patients in the presence or absence of CD161+ cell depletion and in the indicated treatment group (no treatment, anti-CD3/CD28 antibody treatment, anti-IL23/IL-1β antibody treatment, or anti-IL-18/IL-12 antibody treatment).

FIG. 35A is a line graph showing the binding affinity (MFI) of Ab-1, HP-3G10, and hIgG1 control to human CD161 expressed by HEK293 cells in vitro. FIG. 35B is a line graph showing the binding affinity (MFI) of Ab-1, HP-3G10, and hIgG1 control to cyno CD161 expressed by HEK293 cells in vitro.

FIG. 36 is a graph showing the Octet epitope binning assay between Ab-1 and HP-3G10 in sandwich format. A positive signal or shift in wavelength was observed from binding of HP3G10 to human CD161 bound by immobilized Ab-1, which indicated that HP3G10 is not competing with Ab-1 for the same binding site, and therefore belong to different epitope bins.

FIG. 37 shows two FACS plots showing isotype control versus HP-3G10 binding to human CD161 (left) and Ab-1 versus HP-3G10 binding to human CD161 (right).

FIG. 38 is a series of FACS plots showing the phagocytosis of CD161-T cells and CD161+ T cells in the indicated treatment group.

FIG. 39 is a bar graph showing the % phagocytosis of CD161-T cells and CD161+ T cells in the indicated treatment group.

FIG. 40A is a bar graph showing the concentration of GMCSF (pg/mL) produced by human whole blood cells treated with the indicated agent in vitro. All treatments were performed in triplicates per donor. Every data point is expressed as the average of triplicates for each donor. FIG. 40B is a bar graph showing the concentration of IFNγ (pg/mL) produced by human whole blood cells treated with the indicated agent in vitro. All treatments were performed in triplicates per donor. Every data point is expressed as the average of triplicates for each donor. FIG. 40C is a bar graph showing the concentration of IL-2 (pg/mL) produced by human whole blood cells treated with the indicated agent in vitro. All treatments were performed in triplicates per donor. Every data point is expressed as the average of triplicates for each donor. FIG. 40D is a bar graph showing the concentration of IL-12p70 (pg/mL) produced by human whole blood cells treated with the indicated agent in vitro. All treatments were performed in triplicates per donor. Every data point is expressed as the average of triplicates for each donor. FIG. 40E is a bar graph showing the concentration of IL-6 (pg/mL) produced by human whole blood cells treated with the indicated agent in vitro. All treatments were performed in triplicates per donor. Every data point is expressed as the average of triplicates for each donor. FIG. 40F is a bar graph showing the concentration of IL-1β (pg/mL) produced by human whole blood cells treated with the indicated agent in vitro. All treatments were performed in triplicates per donor. Every data point is expressed as the average of triplicates for each donor. FIG. 40G is a bar graph showing the concentration of TNFα (pg/mL) produced by human whole blood cells treated with the indicated agent in vitro. All treatments were performed in triplicates per donor. Every data point is expressed as the average of triplicates for each donor. FIG. 40H is a bar graph showing the concentration of IL-17A (pg/mL) produced by human whole blood cells treated with the indicated agent in vitro. All treatments were performed in triplicates per donor. Every data point is expressed as the average of triplicates for each donor. FIG. 40I is a bar graph showing the concentration of IL-4 (pg/mL) produced by human whole blood cells treated with the indicated agent in vitro. All treatments were performed in triplicates per donor. Every data point is expressed as the average of triplicates for each donor. FIG. 40J is a bar graph showing the concentration of IL-10 (pg/mL) produced by human whole blood cells treated with the indicated agent in vitro. All treatments were performed in triplicates per donor. Every data point is expressed as the average of triplicates for each donor. “Ab-1” in FIGS. 40A-40J refers to afucosylated Ab-1.

FIG. 41A is a bar graph showing the log 2 fold change in IL-17F from Crohn's disease patient-derived colonic biopsies treated with the indicated agent (Ab-1, anti-IL-17A antibody, anti-TNF-α antibody, anti-IL-17A/F antibody, or anti-IgG1 control) antibody. FIG. 41B is a bar graph showing the log 2 fold change in IL-6 from Crohn's disease patient-derived colonic biopsies treated with the indicated agent (Ab-1, anti-IL-17A antibody, anti-TNF-α antibody, anti-IL-17A/F antibody, or anti-IgG1 control) antibody. FIG. 41C is a bar graph showing the log 2 fold change in IL-22 from Crohn's disease patient-derived colonic biopsies treated with the indicated agent (Ab-1, anti-IL-17A antibody, anti-TNF-α antibody, anti-IL-17A/F antibody, or anti-IgG1 control) antibody. FIG. 41D is a bar graph showing the log 2 fold change in IFNγ from Crohn's disease patient-derived colonic biopsies treated with the indicated agent (Ab-1, anti-IL-17A antibody, anti-TNF-α antibody, anti-IL-17A/F antibody, or anti-IgG1 control) antibody. FIG. 41E is a bar graph showing the log 2 fold change in TNFα from Crohn's disease patient-derived colonic biopsies treated with the indicated agent (Ab-1, anti-IL-17A antibody, anti-TNF-α antibody, anti-IL-17A/F antibody, or anti-IgG1 control) antibody. FIG. 41F is a bar graph showing the log 2 fold change in GM-CSF from Crohn's disease patient-derived colonic biopsies treated with the indicated agent (Ab-1, anti-IL-17A antibody, anti-TNF-α antibody, anti-IL-17A/F antibody, or anti-IgG1 control) antibody.

### 5. DETAILED DESCRIPTION

The inventors have, inter alia, engineered novel proteins that specifically bind CD161. Accordingly, the novel CD161 binding proteins disclosed herein are good candidates for the treatment of diseases (e.g., pro-inflammatory diseases (e.g., autoimmune diseases), cancer), diagnostics, etc. The inventors have further discovered, inter alia, that CD161 expressing immune cells (e.g., activated immune cells (e.g., T cells)) can be pathogenic, e.g., expressing higher levels of proinflammatory cytokines (e.g., within the context of a proinflammatory (e.g., autoimmune) disease). Without wishing to be bound by theory, in some embodiments, the CD16 binding proteins (e.g., anti-CD161 antibodies) described herein function to, inter alia, deplete CD161 expressing immune cells (e.g., pathogenic immune cells (e.g., activated immune cells (e.g., T cells))), thereby, e.g., reducing the level of one or more proinflammatory cytokine (e.g., reducing the level of a plurality of proinflammatory cytokines simultaneously). As such, the CD16 binding proteins (e.g., anti-CD161 antibodies) described herein are useful, inter alia, for the treatment of proinflammatory diseases (e.g., autoimmune diseases). As such, the current disclosure provides CD16 binding proteins (e.g., anti-CD161 antibodies) and their use in, inter alia, pharmaceutical compositions, and methods of treating diseases (e.g., proinflammatory diseases (e.g., autoimmune diseases)).

TABLE OF CONTENTS

| | |
|---|---|
| 5.1 | Definitions |
| 5.2 | CD161 Binding Proteins |
| 5.3 | Ig Constant Regions |
| 5.3.1 | Ig Effector Function |
| 5.3.1.1 | Enhanced Ig Effector Function |
| 5.3.1.2 | Reduced Ig Effector Function |
| 5.3.2 | Promotion of Heterodimerization |
| 5.4 | Conjugates and Fusion Proteins |
| 5.4.1 | Chimeric Antigen Receptors |
| 5.4.2 | T-Cell Engagers |
| 5.4.3 | Half-Life Extension Moieties |
| 5.4.4 | Linkers |
| 5.4.4.1 | Non-peptide Linkers |
| 5.4.4.2 | Peptide Linkers |
| 5.4.5 | Orientation |
| 5.5 | Multimeric CD161 Binding Proteins |
| 5.5.1 | Exemplary Properties of CD161 Binding Proteins |
| 5.5.1.1 | Affinity of CD161 Binding Proteins for CD161 |
| 5.5.1.2 | Inhibition of CD161 Binding to CLEC2D |
| 5.5.1.3 | Depletion of Populations of CD161 Expressing Cells and Cytokines |
| 5.6 | Methods of Making Proteins |
| 5.6.1 | Methods of Making Afucosylated Antibodies |
| 5.7 | Polynucleotides, Vectors, Carriers, & Cells |
| 5.8 | Pharmaceutical Compositions |
| 5.9 | Methods of Use |
| 5.9.1 | Methods of Delivery |
| 5.9.2 | Methods of Inhibiting Binding of CD161 to CLEC2D |
| 5.9.3 | Methods of Treating a Disease |
| 5.9.4 | Methods of Treating a Proinflammatory Disease |
| 5.9.5 | Methods of Treating Autoimmune Diseases |
| 5.9.6 | Methods of Depleting CD161 Expressing Cells |
| 5.9.7 | Methods of Reducing the Level of Proinflammatory Cytokines |
| 5.9.8 | Methods of Treating Cancer |
| 5.10 | Kits |

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

Where proteins are described herein, it is understood that polynucleotides (e.g., RNA or DNA nucleic acid molecules) encoding the proteins are also provided herein.

Where proteins, nucleic acid molecules, vectors, carriers, etc. are described herein, it is understood that isolated forms of the proteins, nucleic acid molecules, vectors, carriers, etc. are also provided herein.

Where proteins, nucleic acid molecules, etc. are described herein, it is understood that recombinant forms of the proteins, nucleic acid molecules, etc. are also provided herein.

Where proteins or sets of proteins are described herein, it is understood that both proteins comprising the primary structure are provided herein as well as proteins folded into their three-dimensional structure (i.e., tertiary or quaternary structure) are provided herein.

As used herein, the term “antibody dependent cell mediated cytotoxicity” or “ADCC” refers to an immune mechanism leading to the lysis of antibody (or an Fc region containing protein) (e.g., an Ig Fc containing fusion protein described herein)-coated target cells by immune effector cells (e.g., NK cells). As used herein, the term “reduced ADCC” and the like refers to either a reduction in the number of target cells that are lysed in a given time, at a given concentration of antibody (or an Ig Fc region containing protein) (e.g., an Fc region containing fusion protein described herein) in the medium surrounding the target cells, by the mechanism of ADCC defined above, and/or an increase in the concentration of antibody (or an Fc region containing protein) (e.g., an Fc containing fusion protein described herein) in the medium surrounding the target cells, required to achieve the lysis of a given number of target cells in a given time, by the mechanism of ADCC defined above. The reduction in ADCC is relative to the ADCC mediated by the same antibody (or an Fc region containing protein) (e.g., an Fc containing fusion protein described herein) produced by the same type of host cells, using the same standard production, purification, formulation and storage methods (which are known to those skilled in the art), but that has not been engineered (e.g., does not comprise one or more amino acid variation, e.g., amino acid substitution, that mediates a decrease in ADCC). For example the reduction in ADCC mediated by an antibody (or an Fc region containing protein) (e.g., an Fc containing fusion protein described herein) comprising in its Fc region an amino acid substitution that reduces ADCC, is relative to the ADCC mediated by the same antibody (or an Fc region containing protein) (e.g., an Fc containing fusion protein described herein) without said amino acid substitution in the Fc region.

As used herein, the term “administering” refers to the physical introduction of an agent, e.g., a therapeutic agent (or a precursor of the therapeutic agent that is metabolized or altered within the body of the subject to produce the therapeutic agent in vivo) (e.g., a CD161 binding protein described herein (e.g., an anti-CD161 antibody described herein)) to a subject, using any of the various methods and delivery systems known to those skilled in the art. Administering can also be performed, for example, once, a plurality of times, and/or over one or more extended periods. The term administering includes self-administration and non-self-administration.

As used herein, the term “affinity” refers to the strength of the binding of one protein (e.g., an Antibody) to another protein (e.g., an Antigen). The affinity of a protein is measured by the dissociation constant Kd, defined as [Antibody]×[Antigen]/[Antibody-Antigen] where [Antibody-Antigen] is the molar concentration of the Antibody-Antigen complex, [Antibody] is the molar concentration of the unbound Antibody and [Antigen] is the molar concentration of the unbound Antigen. The affinity constant Ka is defined by 1/Kd. Standard methods of measuring affinity are known to the person of ordinary skill in the art. Exemplary methods of measuring affinity are described herein, see for example, § 5.5.1.

As used herein, the terms “agent” is used generically to describe any macro or micro molecule. Exemplary moieties include, but are not limited polypeptides, proteins, peptides, polynucleotides (e.g., DNA, RNA), small molecules, carbohydrates, lipids, synthetic polymers (e.g., polymers of PEG) (or any combinations thereof).

As used herein, the term “antibody” or “antibodies” is used in the broadest sense and encompasses various immunoglobulin (lg) (e.g., human Ig (hIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). It is common in the art to refer to an anti-X antibody, wherein X is the antigen (e.g., CD161). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methos known in the art, e.g., recombinant production in host cells, see, e.g., § 5.6; or synthetic production.

As used herein, the term “CD161” refers to the type II transmembrane C-type lectin-like receptor expressed, e.g., by natural killer cells. CD161 is also commonly referred to in the art as Killer cell lectin-like receptor subfamily B member 1 (KLRB1). The amino acid sequence of a reference human CD161 (hCD161) protein is set forth in SEQ ID NO: 1 (UniProt Ref.: Q12918-1).

As used herein, the term “CD161 binding protein” and the like refers to a protein (e.g., an antibody (i.e., an anti-CD161 antibody)) that specifically binds CD161.

As used herein, the term “CLEC2D” or “C-type lectin domain family 2 member D” refers to the C type lectin receptor that, inter alia, binds histones released upon necrotic cell death. The amino acid sequence of a reference human CLEC2D (hCLEC2D) isoform is set forth in SEQ ID NO: 2 (UniProt Ref.: Q9UHP7-1). Multiple isoforms of hCLEC2D are known produced by alternative splicing.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), the entire contents of each of which is incorporated herein by reference for all purposes. Unless otherwise specified, the term “CDR” is a CDR as defined by Kabat et al., J. Biol. Chem. 252, 6609-6616 (1977) and Kabat et al., Sequences of protein of immunological interest. (1991). A person of ordinary skill in the art would be able to determine the CDRs as defined by another scheme, e.g., Chothia, IMGT, using ordinary methods known in the art. The three sequential CDRs of the VH region are typically termed “CDR-H1”, “CDR-H2”, and “CDR-H3” herein. The three sequential CDRs of the VL region are typically termed “CDR-L1”, “CDR-L2”, and “CDR-L3” herein, as is common in the art.

The terms “CH1” and “CH1 region” are used interchangeably herein and refer to the first constant region of an immunoglobulin heavy chain. The amino acid sequence of an exemplary reference IgG1 CH1 region is set forth in SEQ ID NO: 11; and the amino acid sequence of an exemplary reference IgG4 CH1 region is set forth in SEQ ID NO: 24.

The terms “CH2” and “CH2 region” are used interchangeably herein and refer to the second constant region of an immunoglobulin heavy chain. The amino acid sequence of an exemplary reference IgG1 CH2 region is set forth in SEQ ID NO: 13; and the amino acid sequence of an exemplary reference IgG4 CH2 region is set forth in SEQ ID NO: 26.

The terms “CH3” and “CH3 region” are used interchangeably herein and refer to the third constant region of an immunoglobulin heavy chain. The amino acid sequence of an exemplary reference IgG1 CH3 region is set forth in SEQ ID NO: 14; and the amino acid sequence of an exemplary reference IgG4 CH3 region is set forth in SEQ ID NO: 27.

As used herein, the term “conjugation” refers to chemical conjugation of a protein with a moiety (e.g., small molecule, polypeptide, nucleic acid molecule, carbohydrate, lipid, synthetic polymer (e.g., polymers of polyethylene glycol (PEG)), etc.). The moiety can be directly connected to the protein or indirectly connected through a linker, e.g., as described herein. Chemical conjugation methods are well known in the art, as are commercially available conjugation reagents and kits, with detailed instructions for their use readily available from the commercial suppliers.

The terms “constant region” and “constant domain” are used interchangeably herein and refer to a carboxyl terminal portion of a light and/or heavy chain of a full-length antibody which is not directly involved in binding of an antibody to antigen, but which can exhibit various effector functions, such as interaction with an Ig Fc receptor (e.g., Fc gamma receptor). The constant region of an Ig molecule generally has a more conserved amino acid sequence relative to an Ig variable domain.

As used herein, the term “combination” in reference to the administration of at least two agents to a subject or similar terms such as “administering in combination”, “co-administration” or “combination” includes the administration of at least two active agents. The two agents may be formulated in separate pharmaceutical formulations or in single pharmaceutical formulation. The at least two active agents may be administered simultaneously or consecutively in any order such that, there is a time period while both (or all) active agents overlap in exerting their biological activities. Administering in combination does not require that the agents are administered at the same time, at the same frequency, or by the same route of administration.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the polynucleotide or protein. For example, the polynucleotide or protein can be recombinantly produced or chemically synthesized.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an Ig Fc region or a protein comprising an Ig Fc region (e.g., a full-length antibody) refers to those biological activities attributable to the Ig Fc region of a typical full-length antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and C1q binding.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, the entire contents of each of which is incorporated herein by reference for all purposes.

As used herein, the term “Fab” refers to an antigen binding domain that comprises a Fab heavy chain that comprises from N- to C-terminus a VH region and a CH1 region; and a light chain comprising from N- to C-terminus a VL region and a CL region; and wherein the Fab heavy chain and the light chain associate to form an antigen binding domain.

The term “Fab-Fc” as used herein refers to an antibody that comprises a Fab operably linked to an Fc region.

As used herein, the term “Fc region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

As used herein, the terms “first” and “second” with respect to Fc regions etc., are used for convenience of distinguishing when there is more than one of each type of moiety. Use of these terms is not intended to confer a specific order or orientation in the protein unless explicitly so stated. For example, an antibody described herein (e.g., in the case of a full-length antibody) may contain two Fc regions that associate e.g., via one or more covalent (e.g., disulfide) bond.

As used herein, the term “framework region” or “FR region” refers to the amino acid residues that are part of the variable region of an antibody, but are not part of the CDRs (e.g., using the Kabat definition of CDRs).

As used herein, the term “full-length antibody” refers to an antibody having a structure substantially similar to a native antibody structure (i) a first immunoglobulin (lg) light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, alternative glycosylation patterns, etc.

The term “functional variant” as used herein in reference to a protein refers to a protein that comprises at least one but no more than 15%, not more than 12%, no more than 10%, no more than 8% amino acid modification (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein. For example, a functional variant of an antibody that specifically binds CD161 can refer to the antibody that specifically binds CD161 comprising one or more amino acid substitution as compared to a reference antibody that retains the ability to specifically bind CD161.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. In some embodiments, the functional fragment protein comprises at least one but no more than 15%, not more than 12%, no more than 10%, no more than 8% amino acid deletion compared to the amino acid sequence of a reference protein. For example, a functional fragment of an antibody that specifically binds CD161 can refer to a fragment of the antibody that retains the ability to specifically bind CD161.

As used herein, the term “fuse” and grammatical equivalents thereof refers to the operable connection of at least a first polypeptide to a second polypeptide, wherein the first and second polypeptides are not naturally found operably connected together. For example, the first and second polypeptides are derived from different proteins and/or are from different organisms. The term fuse encompasses both a direct connection of the at least two polypeptides through a peptide bond, and the indirect connection through a linker (e.g., a peptide linker).

As used herein, the term “fusion protein” and grammatical equivalents thereof refer to a protein that comprises at least one polypeptide operably connected to another polypeptide, wherein the first and second polypeptides are not naturally found operably connected together. For example, the first and second polypeptides of the fusion protein are each derived from different proteins and/or are from heterologous organisms. For the sake of clarity, it will be understood that neither the first nor second polypeptide is required to be a full-length protein (e.g., a full-length naturally occurring protein). For example, the first and/or second polypeptide can comprise or consist of fragments (e.g., functional fragments or domains of full-length proteins (e.g., engineered, naturally occurring). The at least two polypeptides of the fusion protein can be directly operably connected through a peptide bond; or can be indirectly operably connected through a linker (e.g., a peptide linker). Thus, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, nucleic acid molecule, carbohydrate, lipid, synthetic polymer (e.g., polymers of PEG), etc.) that when conjugated or otherwise operably connected (e.g., fused) to a protein (the subject protein), increases the half-life of the subject protein in vivo when administered to a subject (e.g., a human subject). The pharmacokinetic properties of the protein can be evaluated utilizing in vivo models known in the art.

As used herein, the term “half-life extension polypeptide” or “half-life extension protein” refers to a protein that when operably connected to another protein (the subject protein), increases the half-life of the subject protein in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature.

As used herein, the term “heavy chain” refers to the portion of an immunoglobulin (e.g., a human Ig) that typically comprises from N- to C-terminus a heavy chain variable region (VH), a CH1 region, a hinge region, a CH2 region, and a CH3 region. The constant regions of the heavy chain (i.e., the CH1 region, the hinge region, the CH2 region, and the CH3 region) can be any distinct isotype, for example, human alpha (α), delta (δ), epsilon (ε), gamma (γ), and mu (μ), based on the amino acid sequence of the constant domain, which give rise to the IgA, IgD, IgE, IgG, and IgM classes of human antibodies, respectively, including subclasses of IgG, e.g., IgG₁, IgG₂, IgG₃, and IgG₄. As used herein, the term “heavy chain” when used in reference to a human antibody can refer to any distinct type, e.g., alpha (α), delta (δ), epsilon (ε), gamma (γ), and mu (μ), based on the amino acid sequence of the constant domain, which give rise to human IgA, IgD, IgE, IgG, and IgM classes of antibodies, respectively, including subclasses of human IgG, e.g., IgG₁, IgG₂, IgG₃, and IgG₄.

The terms “hinge” or “hinge region” are used interchangeably herein and refer to the hinge region of an immunoglobulin heavy chain. The amino acid sequence of an exemplary reference IgG1 hinge region is set forth in SEQ ID NO: 12; and the amino acid sequence of an exemplary reference IgG4 hinge region is set forth in SEQ ID NO: 25.

As used herein, the term “isolated” with reference to a protein or polynucleotide refers to a protein or polynucleotide that is substantially free of other cellular components with which it is associated in the natural state.

As used herein, the term “moiety” is used generically to describe any macro or micro molecule that can be incorporated into a fusion protein described herein. The moieties of a fusion protein are operably connected. Exemplary moieties include, but are not limited protein, polypeptides, polynucleotides (e.g., DNA, RNA), small molecules, carbohydrates, lipids, synthetic polymers (e.g., polymers of PEG). In some embodiments, the moiety is a protein.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87:2264-2268, modified as in Karlin S & Altschul S F (1993) PNAS 90:5873-5877, each of which is herein incorporated by reference in its entirety. Such an algorithm is incorporated into the NBLAST and XBLAST programs of Altschul S F et al., (1990) J Mol Biol 215:403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25:3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the terms “protein” and “polypeptide” refer to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, a protein is folded into its three-dimensional structure. Where proteins are contemplated herein, it should be understood that proteins comprising the primary structure are provided herein as well as proteins folded into their three-dimensional structure (i.e., tertiary or quaternary structure) are provided herein.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

The term “scFv” or “single chain variable fragment” as used herein refers an antigen binding protein that comprises a VH operably connected (e.g., via a peptide linker) to a VL. In some embodiments, the VH is operably connected to the VL via a peptide linker. The components of the scFv can be in any orientation, for example, the scFv can comprise from N- to C-terminus a VH, a peptide linker, and a VL; or from N- to C-terminus a VL, a peptide linker, and a VH.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “specifically binds” with reference to two proteins refers to a preferential interaction, i.e., significantly higher binding affinity, between a first protein (e.g., an antibody) and a second protein (e.g., an antigen) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human CD161 may be cross reactive with CD161 of another species (e.g., cynomolgus, murine, etc.) and still be considered herein to specifically bind human CD161. The same term can be utilized to describe the preferential binding of non-protein agents (e.g., binding of a small molecule to a protein).

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of an agent (e.g., therapeutic agent) refers to any amount of the agent (e.g., therapeutic agent) that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of an agent (e.g., therapeutic agent) to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a composition as described herein. The term treating includes prophylactic use.

As used herein, the term “modification,” with reference to a polynucleotide, refers to a polynucleotide that comprises at least one substitution, alteration, inversion, addition, or deletion of nucleotide compared to a reference polynucleotide (e.g., one or more amino acid substitutions). Modifications can include the inclusion of non-naturally occurring nucleotide residues. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues. Naturally occurring amino acid derivatives are not considered modified amino acids for purposes of determining percent identity of two amino acid sequences. For example, a naturally occurring modification of a glutamate amino acid residue to a pyroglutamate amino acid residue would not be considered an amino acid modification for purposes of determining percent identity of two amino acid sequences. Further, for example, a naturally occurring modification of a glutamate amino acid residue to a pyroglutamate amino acid residue would not be considered an amino acid “modification” as defined herein.

As used herein, the term “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂-Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

### 5.2 CD161 Binding Proteins

As described above, provided herein are, inter alia, proteins (e.g., antibodies (and functional fragments and functional variants thereof) that specifically bind CD161 (e.g., hCD161).

CD161 is a C-type lectin-like receptor, which is expressed e.g., on NK cells and subsets of both CD4+ and CD8+ T cells. CD161 binds, e.g., CLEC2D. CLEC2D is expressed, e.g., on the surface of both malignant cells and immune cells including germinal center B cells, activated T cells and tumor associated macrophages. CD161 is also expressed on pathogenic effector Th2 (peTh2 cells), TH2A cells, and ILC2 cells (innate like cell-2) that are involved in Th2 mediated diseases (including, e.g., allergies, asthma and eosinophilic diseases). See, e.g., Nakayama T, Hirahara K, Onodera A, Endo Y, Hosokawa H, Shinoda K, Tumes D J, Okamoto Y. Th2 Cells in Health and Disease. Annu Rev Immunol. 2017 Apr. 26; 35:53-84. doi:10.1146/annurev-immunol-051116-052350. Epub 2016 Nov. 28. PMID: 27912316; Wambre E, Bajzik V, DeLong J H, et al. A phenotypically and functionally distinct human TH2 cell subpopulation is associated with allergic disorders. Sci Transl Med. 2017; 9(401):eaam9171. doi:10.1126/scitranslmed.aam9171; and Morgan D M, Ruiter B, Smith N P, et al. Clonally expanded, GPR15-expressing pathogenic effector TH2 cells are associated with eosinophilic esophagitis. Sci Immunol. 2021; 6(62):eabi5586. doi:10.1126/sciimmunol.abi5586, the entire contents of each of which is incorporated herein by reference for all purposes.

The amino acid sequence of a reference hCD161 protein is set forth in SEQ ID NO: 1. Multiple isoforms of hCLEC2D are known produced by alternative splicing, with isoform 1 being the only isoform predominantly expressed at the cell surface. The amino acid sequence of a reference hCLEC2D isoform 1 protein is set forth in SEQ ID NO: 2. See Table 1, herein.

TABLE 1
The Amino Acid Sequence of a Reference hCLEC2D and hCD161 Protein.

| | | SEQ ID |
|---|---|---|
| Description | Amino Acid Sequence | NO |
| hCD161 | MDQQAIYAELNLPTDSGPESSSPSSLPRDVCQGSPWHOFALKLSCA | 1 |
| UniProt | GIILLVLVVTGLSVSVTSLIQKSSIEKCSVDIQQSRNKTTERPGLL | |
| Accession | NCPIYWQQLREKCLLFSHTVNPWNNSLADCSTKESSLLLIRDKDEL | |
| Q12918-1 | IHTQNLIRDKAILFWIGLNFSLSEKNWKWINGSFLNSNDLEIRGDA | |
| | KENSCISISQTSVYSEYCSTEIRWICQKELTPVRNKVYPDS | |
| hCLEC2D | MHDSNNVEKDITPSELPANPGCLHSKEHSIKATLIWRLFFLIMFLT | 2 |
| UniProt | IIVCGMVAALSAIRANCHQEPSVCLQAACPESWIGFORKCFYFSDD | |
| Accession | TKNWTSSQRFCDSQDADLAQVESFQELNFLLRYKGPSDHWIGLSRE | |
| Q9UHP7-1 | QGQPWKWINGTEWTRQFPILGAGECAYLNDKGASSARHYTERKWIC | |
| | SKSDIHV | |

In some embodiments, the CD161 binding protein comprises an antibody. In some embodiments, the CD161 binding protein comprises a full-length antibody, Fab, Fab′, F(ab′)2, Fab-Fc, scFv, scFv-Fc, (scFv)₂-Fc, Fv, a single domain antibody (sdAb) (e.g., a VHH), a sdAb-Fc (e.g., a VHH-Fc), (sdAb)₂ (e.g., a (VHH)₂, or a (sdAb)₂-Fc (e.g., (VHH)₂-Fc). In some embodiments, the CD161 binding protein comprises a full-length antibody, Fab, Fab′, F(ab′)₂, Fab-Fc, scFv, scFv-Fc, (scFv)₂-Fc, sdAb-Fc (e.g., a VHH-Fc), or (sdAb)₂-Fc (e.g., (VHH)₂-Fc). In some embodiments, the CD161 binding protein comprises a full-length antibody. In some embodiments, the CD161 binding protein comprises a Fab. In some embodiments, the antibody comprises a F(ab′)₂. In some embodiments, the CD161 binding protein comprises a Fab-Fc. In some embodiments, the antibody comprises a scFv-Fc. In some embodiments, the CD161 binding protein comprises a (scFv)₂-Fc. In some embodiments, the CD161 binding protein comprises a sdAb-Fc (e.g., a VHH-Fc). In some embodiments, the CD161 binding protein comprises a (sdAb)₂-Fc (e.g., (VHH)₂-Fc). In some embodiments, the CD161 binding protein comprises a single domain antibody. In some embodiments, the CD161 binding protein comprises a VHH.

In some embodiments, the CD161 binding protein comprises an antibody. In some embodiments, the antibody is an IgG1, IgG2, IgG3, or IgG4 antibody. In some embodiments, the antibody is an IgG1 or IgG4 antibody. In some embodiments, the antibody is an IgG1 antibody. In some embodiments, the antibody is an IgG4 antibody. In some embodiments, the antibody is an IgG1, IgG2, IgG3, or IgG4 antibody. In some embodiments, the antibody is an IgG1 or IgG4 antibody. In some embodiments, the antibody is an IgG1 antibody. In some embodiments, the antibody is an IgG4 antibody.

In specific preferred embodiments, the CD161 binding protein is a full length antibody. In specific preferred embodiments, the CD161 binding protein is a full length afucosylated antibody. In specific preferred embodiments, the CD161 binding protein comprises a full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

In specific preferred embodiments, the CD161 binding protein is an afucosylated full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

In specific preferred embodiments, the CD161 binding protein is a monospecific afucosylated full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

In some embodiments, the CD161 binding protein is monospecific. In some embodiments, the CD161 binding protein is multispecific (e.g., bispecific, trispecific). In some embodiments, the CD161 binding protein is multispecific comprising at least one moiety that specifically binds another antigen (i.e., not CD161). In some embodiments, the CD161 binding protein is bispecific comprising at least one moiety that specifically binds another antigen (i.e., not CD161).

In some embodiments, the CD161 binding proteins (e.g., described herein) (or conjugates or fusions comprising the same) are multimeric (e.g., dimeric, trimeric, tetrameric, etc.) proteins comprising at least two, three, or four polypeptides.

In some embodiments, the CD161 binding protein comprises at least two, three, or four polypeptides. In some embodiments, the CD161 binding protein is dimeric (i.e., comprises two polypeptides). In some embodiments, the CD161 binding protein is trimeric (i.e., comprises three polypeptides). In some embodiments, the CD161 binding protein is tetrameric (i.e., comprises four polypeptides).

In some embodiments, two of the polypeptides associate via covalent or non-covalent interactions. In some embodiments, two of the polypeptides associate via at least one covalent interaction. In some embodiments, two of the polypeptides associate via one or more disulfide bond. In some embodiments, two of the polypeptides associate via 1, 2, 3, 4, or more disulfide bonds.

The amino acid sequence of hCD161 binding proteins (e.g., anti-hCD161 antibodies) of the present disclosure is provided in Table 2. The CDRs of the antibodies in Table 2, are denoted according to Kabat, Chothia, AbM, and IMGT. A person of ordinary skill in the art would be able to determine the CDRs as defined by another scheme, e.g., contact, using ordinary methods known in the art.

TABLE 2
The Amino Acid Sequence of hCD161 Binding Proteins.

| | | | SEQ | |
|---|---|---|---|---|
| | Description | | ID NO | Amino Acid Sequence |
| Ab-1 | Kabat | CDR-H1 | 3 | HYAIA |
| | | CDR-H2 | 4 | GIIPEFGNANYAQKFQG |
| | | CDR-H3 | 5 | EGLYYRTSNYGMDV |
| | | CDR-L1 | 6 | RASQSVSSNLA |
| | | CDR-L2 | 7 | GASTRVT |
| | | CDR-L3 | 8 | QQLSPWPWT |
| | Chothia | CDR-H1 | 59 | GGTFSHY |
| | | CDR-H2 | 60 | IPEFGN |
| | | CDR-H3 | 5 | EGLYYRTSNYGMDV |
| | | CDR-L1 | 6 | RASQSVSSNLA |
| | | CDR-L2 | 7 | GASTRVT |
| | | CDR-L3 | 8 | QQLSPWPWT |
| | AbM | CDR-H1 | 61 | GGTFSHYAIA |
| | | CDR-H2 | 62 | GIIPEFGNAN |
| | | CDR-H3 | 5 | EGLYYRTSNYGMDV |
| | | CDR-L1 | 6 | RASQSVSSNLA |
| | | CDR-L2 | 7 | GASTRVT |
| | | CDR-L3 | 8 | QQLSPWPWT |
| | IMGT | CDR-H1 | 63 | GGTFSHYA |
| | | CDR-H2 | 64 | IIPEFGNA |
| | | CDR-H3 | 65 | AREGLYYRTSNYGMDV |
| | | CDR-L1 | 66 | QSVSSN |
| | | CDR-L2 | N/A | GAS |
| | | CDR-L3 | 8 | QQLSPWPWT |
| | VH | | 9 | QVQLVQSGAEVKKPGSSVKVSCKASGGTFSH |
| | | | | YAIAWVRQAPGQGLEWMGGIIPEFGNANYAQ |
| | | | | KFQGRVTITADESTSTAYMELSSLRSEDTAV |
| | | | | YYCAREGLYYRTSNYGMDVWGQGTTVTVSS |
| | VL | | 10 | EIVLTQSPATLSVSPGERATLSCRASQSVSS |
| | | | | NLAWYQQKPGQAPRLLIYGASTRVTGIPARF |
| | | | | SGSGSGTEFTLTISSLQSEDFAVYYCQQLSP |
| | | | | WPWTFGGGTKVEIK |
| | HC | | 67 | QVQLVQSGAEVKKPGSSVKVSCKASGGTFSH |
| | | | | YAIAWVRQAPGQGLEWMGGIIPEFGNANYAQ |
| | | | | KFQGRVTITADESTSTAYMELSSLRSEDTAV |
| | | | | YYCAREGLYYRTSNYGMDVWGQGTTVTVSSA |
| | | | | STKGPSVFPLAPSSKSTSGGTAALGCLVKDY |
| | | | | FPEPVTVSWNSGALTSGVHTFPAVLQSSGLY |
| | | | | SLSSVVTVPSSSLGTQTYICNVNHKPSNTKV |
| | | | | DKKVEPKSCDKTHTCPPCPAPELLGGPSVFL |
| | | | | FPPKPKDTLMISRTPEVTCVVVDVSHEDPEV |
| | | | | KFNWYVDGVEVHNAKTKPREEQYNSTYRVVS |
| | | | | VLTVLHQDWLNGKEYKCKVSNKALPAPIEKT |
| | | | | ISKAKGQPREPQVYTLPPSRDELTKNQVSLT |
| | | | | CLVKGFYPSDIAVEWESNGQPENNYKTTPPV |
| | | | | LDSDGSFFLYSKLTVDKSRWQQGNVFSCSVM |
| | | | | HEALHNHYTQKSLSLSPGK |
| | LC | | 68 | EIVLTQSPATLSVSPGERATLSCRASQSVSS |
| | | | | NLAWYQQKPGQAPRLLIYGASTRVTGIPARF |
| | | | | SGSGSGTEFTLTISSLQSEDFAVYYCQQLSP |
| | | | | WPWTFGGGTKVEIKRTVAAPSVFIFPPSDEQ |
| | | | | LKSGTASVVCLLNNFYPREAKVQWKVDNALQ |
| | | | | SGNSQESVTEQDSKDSTYSLSSTLTLSKADY |
| | | | | EKHKVYACEVTHQGLSSPVTKSFNRGEC |

In specific embodiments, the heavy chain lacks the C-terminal lysine at position 448, EU numbering according to Kabat. In some embodiments, the heavy lacks the C-terminal glycine and lysine at positions 447 and 448, respectively, EU numbering according to Kabat.

In some embodiments, the CD161 binding protein (e.g., anti-CD161 antibody) comprises a VH that comprises: a CDR-H1, a CDR-H2, and a CDR-H3. In some embodiments, the CD161 binding protein comprises a VL that comprises: CDR-L1, CDR-L2, and CDR-L3. In some embodiments, the CD161 binding protein comprises a VH that comprises: a CDR-H1, a CDR-H2, and a CDR-H3; and a VL that comprises: CDR-L1, CDR-L2, and CDR-L3.

In some embodiments, the CD161 binding protein comprises a CD161 binding protein provided in Table 2.

In some embodiments, the amino acid sequence of VH CDR 1 comprises the amino acid sequence of a VH CDR1 of a VH set forth in Table 2, or the amino acid of a VH CDR1 of a VH set forth in Table 2, comprising 1, 2, or 3 amino acid variations (e.g., substitution, deletion, addition, etc.); the amino acid sequence of VH CDR2 comprises the amino acid sequence of a VH CDR2 of a VH set forth in Table 2, or the amino acid sequence of a VH CDR2 of a VH set forth in Table 2 comprising 1, 2, or 3 amino acid variations (e.g., substitution, deletion, addition, etc.); the amino acid sequence of VH CDR3 comprises of a VH CDR3 of a VH set forth in Table 2, or the amino acid sequence of a VH CDR3 of a VH set forth in Table 2 comprising 1, 2, or 3 amino acid variations (e.g., substitution, deletion, addition, etc.); the amino acid sequence of VL CDR1 comprises the amino acid sequence of a VL CDR1 of a VL set forth in Table 2, or the amino acid sequence of a VL CDR1 of a VL set forth in Table 2 comprising 1, 2, or 3 amino acid variations (e.g., substitution, deletion, addition, etc.); the amino acid sequence of VL CDR2 comprises the amino acid sequence of a VL CDR2 of a VL set forth in Table 2, or the amino acid sequence of a VL CDR2 of a VL set forth in Table 2 comprising 1, 2, or 3 amino acid variations (e.g., substitution, deletion, addition, etc.); and the amino acid sequence of VL CDR3 comprises the amino acid sequence of a VL CDR3 of a VL set forth in Table 2, or the amino acid sequence of a VL CDR3 of a VL set forth in Table 2 comprising 1, 2, or 3 amino acid variations (e.g., substitution, deletion, addition, etc.).

In some embodiments, the amino acid sequence of VH CDR 1 comprises the amino acid sequence of a VH CDR1 of a VH set forth in Table 2, or the amino acid of a VH CDR1 of a VH set forth in Table 2, comprising no more than 1, 2, or 3 amino acid variations (e.g., substitution, deletion, addition, etc.); the amino acid sequence of VH CDR2 comprises the amino acid sequence of a VH CDR2 of a VH set forth in Table 2, or the amino acid sequence of a VH CDR2 of a VH set forth in Table 2 comprising no more than 1, 2, or 3 amino acid variations (e.g., substitution, deletion, addition, etc.); the amino acid sequence of VH CDR3 comprises of a VH CDR3 of a VH set forth in Table 2, or the amino acid sequence of a VH CDR3 of a VH set forth in Table 2 comprising no more than 1, 2, or 3 amino acid variations (e.g., substitution, deletion, addition, etc.); the amino acid sequence of VL CDR1 comprises the amino acid sequence of a VL CDR1 of a VL set forth in Table 2, or the amino acid sequence of a VL CDR1 of a VL set forth in Table 2 comprising no more than 1, 2, or 3 amino acid variations (e.g., substitution, deletion, addition, etc.); the amino acid sequence of VL CDR2 comprises the amino acid sequence of a VL CDR2 of a VL set forth in Table 2, or the amino acid sequence of a VL CDR2 of a VL set forth in Table 2 comprising no more than 1, 2, or 3 amino acid variations (e.g., substitution, deletion, addition, etc.); and the amino acid sequence of VL CDR3 comprises the amino acid sequence of a VL CDR3 of a VL set forth in Table 2, or the amino acid sequence of a VL CDR3 of a VL set forth in Table 2 comprising no more than 1, 2, or 3 amino acid variations (e.g., substitution, deletion, addition, etc.).

In some embodiments, the amino acid sequence of VH CDR 1 comprises the amino acid sequence of a VH CDR1 of a VH set forth in Table 2; the amino acid sequence of VH CDR2 comprises the amino acid sequence of a VH CDR2 of a VH set forth in Table 2; the amino acid sequence of VH CDR3 comprises of a VH CDR3 of a VH set forth in Table 2; the amino acid sequence of VL CDR1 comprises the amino acid sequence of a VL CDR1 of a VL set forth in Table 2; the amino acid sequence of VL CDR2 comprises the amino acid sequence of a VL CDR2 of a VL set forth in Table 2; and the amino acid sequence of VL CDR3 comprises the amino acid sequence of a VL CDR3 of a VL set forth in Table 2.

In some embodiments, the amino acid sequence of VH CDR1 comprises the amino acid of a VH CDR1 of a VH set forth in Table 2, comprising no more than 1, 2, or 3 amino acid variations (e.g., substitution, deletion, addition, etc.); the amino acid sequence of a VH CDR2 of a VH set forth in Table 2 comprising no more than 1, 2, or 3 amino acid variations (e.g., substitution, deletion, addition, etc.); the amino acid sequence of a VH CDR3 of a VH set forth in Table 2 comprising no more than 1, 2, or 3 amino acid variations (e.g., substitution, deletion, addition, etc.); the amino acid sequence of a VL CDR1 of a VL set forth in Table 2 comprising no more than 1, 2, or 3 amino acid variations (e.g., substitution, deletion, addition, etc.); the amino acid sequence of a VL CDR2 of a VL set forth in Table 2 comprising no more than 1, 2, or 3 amino acid variations (e.g., substitution, deletion, addition, etc.); and the amino acid sequence of a VL CDR3 of a VL set forth in Table 2 comprising no more than 1, 2, or 3 amino acid variations (e.g., substitution, deletion, addition, etc.).

In some embodiments, the amino acid sequence of VH CDR1 consists of the amino acid sequence of a VH CDR1 of a VH set forth in Table 2, or the amino acid of a VH CDR1 of a VH set forth in Table 2, comprising 1, 2, or 3 amino acid variations (e.g., substitution, deletion, addition, etc.); the amino acid sequence of VH CDR2 consists of the amino acid sequence of a VH CDR2 of a VH set forth in Table 2, or the amino acid sequence of a VH CDR2 of a VH set forth in Table 2 comprising 1, 2, or 3 amino acid variations (e.g., substitution, deletion, addition, etc.); the amino acid sequence of VH CDR3 consists of a VH CDR3 of a VH set forth in Table 2, or the amino acid sequence of a VH CDR3 of a VH set forth in Table 2 comprising 1, 2, or 3 amino acid variations (e.g., substitution, deletion, addition, etc.); the amino acid sequence of VL CDR1 consists of the amino acid sequence of a VL CDR1 of a VL set forth in Table 2, or the amino acid sequence of a VL CDR1 of a VL set forth in Table 2 comprising 1, 2, or 3 amino acid variations (e.g., substitution, deletion, addition, etc.); the amino acid sequence of VL CDR2 consists of the amino acid sequence of a VL CDR2 of a VL set forth in Table 2, or the amino acid sequence of a VL CDR2 of a VL set forth in Table 2 comprising 1, 2, or 3 amino acid variations (e.g., substitution, deletion, addition, etc.); and the amino acid sequence of VL CDR3 consists of the amino acid sequence of a VL CDR3 of a VL set forth in Table 2, or the amino acid sequence of a VL CDR3 of a VL set forth in Table 2 comprising 1, 2, or 3 amino acid variations (e.g., substitution, deletion, addition, etc.).

In some embodiments, the amino acid sequence of VH CDR1 consists of the amino acid sequence of a VH CDR1 of a VH set forth in Table 2, or the amino acid of a VH CDR1 of a VH set forth in Table 2, comprising no more than 1, 2, or 3 amino acid variations (e.g., substitution, deletion, addition, etc.); the amino acid sequence of VH CDR2 consists of the amino acid sequence of a VH CDR2 of a VH set forth in Table 2, or the amino acid sequence of a VH CDR2 of a VH set forth in Table 2 comprising no more than 1, 2, or 3 amino acid variations (e.g., substitution, deletion, addition, etc.); the amino acid sequence of VH CDR3 consists of a VH CDR3 of a VH set forth in Table 2, or the amino acid sequence of a VH CDR3 of a VH set forth in Table 2 comprising no more than 1, 2, or 3 amino acid variations (e.g., substitution, deletion, addition, etc.); the amino acid sequence of VL CDR1 consists of the amino acid sequence of a VL CDR1 of a VL set forth in Table 2, or the amino acid sequence of a VL CDR1 of a VL set forth in Table 2 comprising no more than 1, 2, or 3 amino acid variations (e.g., substitution, deletion, addition, etc.); the amino acid sequence of VL CDR2 consists of the amino acid sequence of a VL CDR2 of a VL set forth in Table 2, or the amino acid sequence of a VL CDR2 of a VL set forth in Table 2 comprising no more than 1, 2, or 3 amino acid variations (e.g., substitution, deletion, addition, etc.); and the amino acid sequence of VL CDR3 consists of the amino acid sequence of a VL CDR3 of a VL set forth in Table 2, or the amino acid sequence of a VL CDR3 of a VL set forth in Table 2 comprising no more than 1, 2, or 3 amino acid variations (e.g., substitution, deletion, addition, etc.).

In some embodiments, the amino acid sequence of VH CDR1 consists of the amino acid sequence of a VH CDR1 of a VH set forth in Table 2; the amino acid sequence of VH CDR2 consists of the amino acid sequence of a VH CDR2 of a VH set forth in Table 2; the amino acid sequence of VH CDR3 consists of a VH CDR3 of a VH set forth in Table 2; the amino acid sequence of VL CDR1 consists of the amino acid sequence of a VL CDR1 of a VL set forth in Table 2; the amino acid sequence of VL CDR2 consists of the amino acid sequence of a VL CDR2 of a VL set forth in Table 2; and the amino acid sequence of VL CDR3 consists of the amino acid sequence of a VL CDR3 of a VL set forth in Table 2.

In some embodiments, the amino acid sequence of VH CDR1 consists of the amino acid of a VH CDR1 of a VH set forth in Table 2, comprising no more than 1, 2, or 3 amino acid variations (e.g., substitution, deletion, addition, etc.); the amino acid sequence of a VH CDR2 of a VH set forth in Table 2 comprising no more than 1, 2, or 3 amino acid variations (e.g., substitution, deletion, addition, etc.); the amino acid sequence of a VH CDR3 of a VH set forth in Table 2 comprising no more than 1, 2, or 3 amino acid variations (e.g., substitution, deletion, addition, etc.); the amino acid sequence of a VL CDR1 of a VL set forth in Table 2 comprising no more than 1, 2, or 3 amino acid variations (e.g., substitution, deletion, addition, etc.); the amino acid sequence of a VL CDR2 of a VL set forth in Table 2 comprising no more than 1, 2, or 3 amino acid variations (e.g., substitution, deletion, addition, etc.); and the amino acid sequence of a VL CDR3 of a VL set forth in Table 2 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 2; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 2. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH set forth in Table 2; and the amino acid sequence of the VL comprises an amino acid sequence at least 90%, 91%, 92%, 93%, 94%, 95%, 96%, 97%, 98%, 99%, or 100% identical to the amino acid sequence of a VL set forth in Table 2. In some embodiments, the amino acid sequence of the VH comprises an amino acid sequence at least 95%, 96%, 97%, 98%, 99%, or 100% identical to the amino acid sequence of a VH set forth in Table 2; and the amino acid sequence of the VL comprises an amino acid sequence at least 95%, 96%, 97%, 98%, 99%, or 100% identical to the amino acid sequence of a VL set forth in Table 2. In some embodiments, the amino acid sequence of the VH comprises the amino acid sequence of a VH set forth in Table 2; and the amino acid sequence of the VL comprises the amino acid sequence of a VL set forth in Table 2.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 2; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 2. In some embodiments, the amino acid sequence of the VH consists of an amino acid sequence at least 90%, 91%, 92%, 93%, 94%, 95%, 96%, 97%, 98%, 99%, or 100% identical to the amino acid sequence of a VH set forth in Table 2; and the amino acid sequence of the VL consists of an amino acid sequence at least 90%, 91%, 92%, 93%, 94%, 95%, 96%, 97%, 98%, 99%, or 100% identical to the amino acid sequence of a VL set forth in Table 2. In some embodiments, the amino acid sequence of the VH consists of an amino acid sequence at least 95%, 96%, 97%, 98%, 99%, or 100% identical to the amino acid sequence of a VH set forth in Table 2; and the amino acid sequence of the VL consists of an amino acid sequence at least 95%, 96%, 97%, 98%, 99%, or 100% identical to the amino acid sequence of a VL set forth in Table 2. In some embodiments, the amino acid sequence of the VH consists of the amino acid sequence of a VH set forth in Table 2; and the amino acid sequence of the VL consists of the amino acid sequence of a VL set forth in Table 2.

In some embodiments, the CD161 binding protein comprises a light chain (LC) and a heavy chain (HC).

In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 2; and the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 2. In some embodiments, the amino acid sequence of the HC comprises an amino acid sequence at least 90%, 91%, 92%, 93%, 94%, 95%, 96%, 97%, 98%, 99%, or 100% identical to the amino acid sequence of a HC set forth in Table 2; and the amino acid sequence of the LC comprises an amino acid sequence at least 90%, 91%, 92%, 93%, 94%, 95%, 96%, 97%, 98%, 99%, or 100% identical to the amino acid sequence of a LC set forth in Table 2. In some embodiments, the amino acid sequence of the HC comprises an amino acid sequence at least 95%, 96%, 97%, 98%, 99%, or 100% identical to the amino acid sequence of a HC set forth in Table 2; and the amino acid sequence of the LC comprises an amino acid sequence at least 95%, 96%, 97%, 98%, 99%, or 100% identical to the amino acid sequence of a LC set forth in Table 2. In some embodiments, the amino acid sequence of the HC comprises the amino acid sequence of a HC set forth in Table 2; and the amino acid sequence of the LC comprises the amino acid sequence of a LC set forth in Table 2.

In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 2; and the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 2. In some embodiments, the amino acid sequence of the HC consists of an amino acid sequence at least 90%, 91%, 92%, 93%, 94%, 95%, 96%, 97%, 98%, 99%, or 100% identical to the amino acid sequence of a HC set forth in Table 2; and the amino acid sequence of the LC consists of an amino acid sequence at least 90%, 91%, 92%, 93%, 94%, 95%, 96%, 97%, 98%, 99%, or 100% identical to the amino acid sequence of a LC set forth in Table 2. In some embodiments, the amino acid sequence of the HC consists of an amino acid sequence at least 95%, 96%, 97%, 98%, 99%, or 100% identical to the amino acid sequence of a HC set forth in Table 2; and the amino acid sequence of the LC consists of an amino acid sequence at least 95%, 96%, 97%, 98%, 99%, or 100% identical to the amino acid sequence of a LC set forth in Table 2. In some embodiments, the amino acid sequence of the HC consists of the amino acid sequence of a HC set forth in Table 2; and the amino acid sequence of the LC consists of the amino acid sequence of a LC set forth in Table 2.

In some embodiments, the amino acid sequence of VH CDR 1 comprises the amino acid sequence of a VH CDR1 set forth in Table 2, or the amino acid of a VH CDR1 set forth in Table 2, comprising 1, 2, or 3 amino acid variations (e.g., substitution, deletion, addition, etc.); the amino acid sequence of VH CDR2 comprises the amino acid sequence of a VH CDR2 set forth in Table 2, or the amino acid sequence of a VH CDR2 set forth in Table 2 comprising 1, 2, or 3 amino acid variations (e.g., substitution, deletion, addition, etc.); the amino acid sequence of VH CDR3 comprises of a VH CDR3 set forth in Table 2, or the amino acid sequence of a VH CDR3 set forth in Table 2 comprising 1, 2, or 3 amino acid variations (e.g., substitution, deletion, addition, etc.); the amino acid sequence of VL CDR1 comprises the amino acid sequence of a VL CDR1 set forth in Table 2, or the amino acid sequence of a VL CDR1 set forth in Table 2 comprising 1, 2, or 3 amino acid variations (e.g., substitution, deletion, addition, etc.); the amino acid sequence of VL CDR2 comprises the amino acid sequence of a VL CDR2 set forth in Table 2, or the amino acid sequence of a VL CDR2 set forth in Table 2 comprising 1, 2, or 3 amino acid variations (e.g., substitution, deletion, addition, etc.); and the amino acid sequence of VL CDR3 comprises the amino acid sequence of a VL CDR3 set forth in Table 2, or the amino acid sequence of a VL CDR3 set forth in Table 2 comprising 1, 2, or 3 amino acid variations (e.g., substitution, deletion, addition, etc.).

In some embodiments, the amino acid sequence of VH CDR1 comprises the amino acid sequence of a VH CDR1 set forth in Table 2, or the amino acid of a VH CDR1 set forth in Table 2, comprising no more than 1, 2, or 3 amino acid variations (e.g., substitution, deletion, addition, etc.); the amino acid sequence of VH CDR2 comprises the amino acid sequence of a VH CDR2 set forth in Table 2, or the amino acid sequence of a VH CDR2 set forth in Table 2 comprising no more than 1, 2, or 3 amino acid variations (e.g., substitution, deletion, addition, etc.); the amino acid sequence of VH CDR3 comprises of a VH CDR3 set forth in Table 2, or the amino acid sequence of a VH CDR3 set forth in Table 2 comprising no more than 1, 2, or 3 amino acid variations (e.g., substitution, deletion, addition, etc.); the amino acid sequence of VL CDR1 comprises the amino acid sequence of a VL CDR1 set forth in Table 2, or the amino acid sequence of a VL CDR1 set forth in Table 2 comprising no more than 1, 2, or 3 amino acid variations (e.g., substitution, deletion, addition, etc.); the amino acid sequence of VL CDR2 comprises the amino acid sequence of a VL CDR2 set forth in Table 2, or the amino acid sequence of a VL CDR2 set forth in Table 2 comprising no more than 1, 2, or 3 amino acid variations (e.g., substitution, deletion, addition, etc.); and the amino acid sequence of VL CDR3 comprises the amino acid sequence of a VL CDR3 set forth in Table 2, or the amino acid sequence of a VL CDR3 set forth in Table 2 comprising no more than 1, 2, or 3 amino acid variations (e.g., substitution, deletion, addition, etc.).

In some embodiments, the amino acid sequence of VH CDR1 comprises the amino acid sequence of a VH CDR1 set forth in Table 2; the amino acid sequence of VH CDR2 comprises the amino acid sequence of a VH CDR2 set forth in Table 2; the amino acid sequence of VH CDR3 comprises of a VH CDR3 set forth in Table 2; the amino acid sequence of VL CDR1 comprises the amino acid sequence of a VL CDR1 set forth in Table 2; the amino acid sequence of VL CDR2 comprises the amino acid sequence of a VL CDR2 set forth in Table 2; and the amino acid sequence of VL CDR3 comprises the amino acid sequence of a VL CDR3 set forth in Table 2.

In some embodiments, the amino acid sequence of VH CDR 1 comprises the amino acid of a VH CDR1 set forth in Table 2, comprising no more than 1, 2, or 3 amino acid variations (e.g., substitution, deletion, addition, etc.); the amino acid sequence of a VH CDR2 set forth in Table 2 comprising no more than 1, 2, or 3 amino acid variations (e.g., substitution, deletion, addition, etc.); the amino acid sequence of a VH CDR3 set forth in Table 2 comprising no more than 1, 2, or 3 amino acid variations (e.g., substitution, deletion, addition, etc.); the amino acid sequence of a VL CDR1 set forth in Table 2 comprising no more than 1, 2, or 3 amino acid variations (e.g., substitution, deletion, addition, etc.); the amino acid sequence of a VL CDR2 set forth in Table 2 comprising no more than 1, 2, or 3 amino acid variations (e.g., substitution, deletion, addition, etc.); and the amino acid sequence of a VL CDR3 set forth in Table 2 comprising no more than 1, 2, or 3 amino acid variations (e.g., substitution, deletion, addition, etc.).

In some embodiments, the amino acid sequence of VH CDR1 consists of the amino acid sequence of a VH CDR1 set forth in Table 2, or the amino acid of a VH CDR1 set forth in Table 2, comprising 1, 2, or 3 amino acid variations (e.g., substitution, deletion, addition, etc.); the amino acid sequence of VH CDR2 consists of the amino acid sequence of a VH CDR2 set forth in Table 2, or the amino acid sequence of a VH CDR2 set forth in Table 2 comprising 1, 2, or 3 amino acid variations (e.g., substitution, deletion, addition, etc.); the amino acid sequence of VH CDR3 consists of a VH CDR3 set forth in Table 2, or the amino acid sequence of a VH CDR3 set forth in Table 2 comprising 1, 2, or 3 amino acid variations (e.g., substitution, deletion, addition, etc.); the amino acid sequence of VL CDR1 consists of the amino acid sequence of a VL CDR1 set forth in Table 2, or the amino acid sequence of a VL CDR1 set forth in Table 2 comprising 1, 2, or 3 amino acid variations (e.g., substitution, deletion, addition, etc.); the amino acid sequence of VL CDR2 consists of the amino acid sequence of a VL CDR2 set forth in Table 2, or the amino acid sequence of a VL CDR2 set forth in Table 2 comprising 1, 2, or 3 amino acid variations (e.g., substitution, deletion, addition, etc.); and the amino acid sequence of VL CDR3 consists of the amino acid sequence of a VL CDR3 set forth in Table 2, or the amino acid sequence of a VL CDR3 set forth in Table 2 comprising 1, 2, or 3 amino acid variations (e.g., substitution, deletion, addition, etc.).

In some embodiments, the amino acid sequence of VH CDR1 consists of the amino acid sequence of a VH CDR1 set forth in Table 2, or the amino acid of a VH CDR1 set forth in Table 2, comprising no more than 1, 2, or 3 amino acid variations (e.g., substitution, deletion, addition, etc.); the amino acid sequence of VH CDR2 consists of the amino acid sequence of a VH CDR2 set forth in Table 2, or the amino acid sequence of a VH CDR2 set forth in Table 2 comprising no more than 1, 2, or 3 amino acid variations (e.g., substitution, deletion, addition, etc.); the amino acid sequence of VH CDR3 consists of a VH CDR3 set forth in Table 2, or the amino acid sequence of a VH CDR3 set forth in Table 2 comprising no more than 1, 2, or 3 amino acid variations (e.g., substitution, deletion, addition, etc.); the amino acid sequence of VL CDR1 consists of the amino acid sequence of a VL CDR 1 set forth in Table 2, or the amino acid sequence of a VL CDR1 set forth in Table 2 comprising no more than 1, 2, or 3 amino acid variations (e.g., substitution, deletion, addition, etc.); the amino acid sequence of VL CDR2 consists of the amino acid sequence of a VL CDR2 set forth in Table 2, or the amino acid sequence of a VL CDR2 set forth in Table 2 comprising no more than 1, 2, or 3 amino acid variations (e.g., substitution, deletion, addition, etc.); and the amino acid sequence of VL CDR3 consists of the amino acid sequence of a VL CDR3 set forth in Table 2, or the amino acid sequence of a VL CDR3 set forth in Table 2 comprising no more than 1, 2, or 3 amino acid variations (e.g., substitution, deletion, addition, etc.).

In some embodiments, the amino acid sequence of VH CDR1 consists of the amino acid sequence of a VH CDR1 set forth in Table 2; the amino acid sequence of VH CDR2 consists of the amino acid sequence of a VH CDR2 set forth in Table 2; the amino acid sequence of VH CDR3 consists of a VH CDR3 set forth in Table 2; the amino acid sequence of VL CDR1 consists of the amino acid sequence of a VL CDR1 set forth in Table 2; the amino acid sequence of VL CDR2 consists of the amino acid sequence of a VL CDR2 set forth in Table 2; and the amino acid sequence of VL CDR3 consists of the amino acid sequence of a VL CDR3 set forth in Table 2.

In some embodiments, the amino acid sequence of VH CDR1 consists of the amino acid of a VH CDR1 set forth in Table 2, comprising no more than 1, 2, or 3 amino acid variations (e.g., substitution, deletion, addition, etc.); the amino acid sequence of a VH CDR2 set forth in Table 2 comprising no more than 1, 2, or 3 amino acid variations (e.g., substitution, deletion, addition, etc.); the amino acid sequence of a VH CDR3 set forth in Table 2 comprising no more than 1, 2, or 3 amino acid variations (e.g., substitution, deletion, addition, etc.); the amino acid sequence of a VL CDR1 set forth in Table 2 comprising no more than 1, 2, or 3 amino acid variations (e.g., substitution, deletion, addition, etc.); the amino acid sequence of a VL CDR2 set forth in Table 2 comprising no more than 1, 2, or 3 amino acid variations (e.g., substitution, deletion, addition, etc.); and the amino acid sequence of a VL CDR3 set forth in Table 2 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 2; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 2. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH set forth in Table 2; and the amino acid sequence of the VL comprises an amino acid sequence at least 90%, 91%, 92%, 93%, 94%, 95%, 96%, 97%, 98%, 99%, or 100% identical to the amino acid sequence of a VL set forth in Table 2. In some embodiments, the amino acid sequence of the VH comprises an amino acid sequence at least 95%, 96%, 97%, 98%, 99%, or 100% identical to the amino acid sequence of a VH set forth in Table 2; and the amino acid sequence of the VL comprises an amino acid sequence at least 95%, 96%, 97%, 98%, 99%, or 100% identical to the amino acid sequence of a VL set forth in Table 2. In some embodiments, the amino acid sequence of the VH comprises the amino acid sequence of a VH set forth in Table 2; and the amino acid sequence of the VL comprises the amino acid sequence of a VL set forth in Table 2.

In some embodiments, the amino acid sequence of VH CDR 1 comprises the amino acid sequence set forth in SEQ ID NO: 3, or the amino acid sequence set forth in SEQ ID NO: 3 comprising 1, 2, or 3 amino acid variations (e.g., substitution, deletion, addition, etc.); the amino acid sequence of VH CDR2 comprises the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR 1 comprises the amino acid sequence set forth in SEQ ID NO: 3, or the amino acid sequence set forth in SEQ ID NO: 3 comprising no more than 1, 2, or 3 amino acid variations (e.g., substitution, deletion, addition, etc.); the amino acid sequence of VH CDR2 comprises the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 comprises the amino acid sequence set forth in SEQ ID NO: 3; the amino acid sequence of VH CDR2 comprises the amino acid sequence set forth in SEQ ID NO: 4; the amino acid sequence of VH CDR3 comprises the amino acid sequence set forth in SEQ ID NO: 5; the amino acid sequence of VL CDR1 comprises the amino acid sequence set forth in SEQ ID NO: 6; the amino acid sequence of VL CDR2 comprises the amino acid sequence set forth in SEQ ID NO: 7; and the amino acid sequence of VL CDR3 comprises the amino acid sequence set forth in SEQ ID NO: 8.

In some embodiments, the amino acid sequence of VH CDR1 the amino acid sequence set forth in SEQ ID NO: 3 comprising no more than 1, 2, or 3 amino acid variations (e.g., substitution, deletion, addition, etc.); the amino acid sequence of VH CDR2 comprises the amino acid sequence set forth in SEQ ID NO: 4 comprising no more than 1, 2, or 3 amino acid variations (e.g., substitution, deletion, addition, etc.); the amino acid sequence of VH CDR3 comprises the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 3, or the amino acid sequence set forth in SEQ ID NO: 3 comprising 1, 2, or 3 amino acid variations (e.g., substitution, deletion, addition, etc.); the amino acid sequence of VH CDR2 consists of the amino acid sequence set forth in SEQ ID NO: 4, or the amino acid sequence set forth in SEQ ID NO: 4 comprising 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR1 consists of the amino acid sequence set forth in SEQ ID NO: 3, or the amino acid sequence set forth in SEQ ID NO: 3 comprising no more than 1, 2, or 3 amino acid variations (e.g., substitution, deletion, addition, etc.); the amino acid sequence of VH CDR2 consists of the amino acid sequence set forth in SEQ ID NO: 4, or the amino acid sequence set forth in SEQ ID NO: 4 comprising no more than 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 3; the amino acid sequence of VH CDR2 consists of the amino acid sequence set forth in SEQ ID NO: 4; the amino acid sequence of VH CDR3 consists of the amino acid sequence set forth in SEQ ID NO: 5; the amino acid sequence of VL CDR1 consists of the amino acid sequence set forth in SEQ ID NO: 6; the amino acid sequence of VL CDR2 consists of the amino acid sequence set forth in SEQ ID NO: 7; and the amino acid sequence of VL CDR3 consists of the amino acid sequence set forth in SEQ ID NO: 8.

In some embodiments, the amino acid sequence of VH CDR1 consists of the amino acid sequence set forth in SEQ ID NO: 3 comprising no more than 1, 2, or 3 amino acid variations (e.g., substitution, deletion, addition, etc.); the amino acid sequence of VH CDR2 consists of the amino acid sequence set forth in SEQ ID NO: 4 comprising no more than 1, 2, or 3 amino acid variations (e.g., substitution, deletion, addition, etc.); the amino acid sequence of VH CDR3 consists of the amino acid sequence set forth in SEQ ID NO: 5 comprising no more than 1, 2, or 3 amino acid variations (e.g., substitution, deletion, addition, etc.); the amino acid sequence of VL CDR 1 consists of the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8 comprising no more than 1, 2, or 3 amino acid variations (e.g., substitution, deletion, addition, etc.).

In some embodiments, the amino acid sequence of VH CDR 1 comprises the amino acid sequence set forth in SEQ ID NO: 59, or the amino acid sequence set forth in SEQ ID NO: 59 comprising 1, 2, or 3 amino acid variations (e.g., substitution, deletion, addition, etc.); the amino acid sequence of VH CDR2 comprises the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR 1 comprises the amino acid sequence set forth in SEQ ID NO: 59, or the amino acid sequence set forth in SEQ ID NO: 59 comprising no more than 1, 2, or 3 amino acid variations (e.g., substitution, deletion, addition, etc.); the amino acid sequence of VH CDR2 comprises the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR 1 comprises the amino acid sequence set forth in SEQ ID NO: 59; the amino acid sequence of VH CDR2 comprises the amino acid sequence set forth in SEQ ID NO: 60; the amino acid sequence of VH CDR3 comprises the amino acid sequence set forth in SEQ ID NO: 5; the amino acid sequence of VL CDR1 comprises the amino acid sequence set forth in SEQ ID NO: 6; the amino acid sequence of VL CDR2 comprises the amino acid sequence set forth in SEQ ID NO: 7; and the amino acid sequence of VL CDR3 comprises the amino acid sequence set forth in SEQ ID NO: 8.

In some embodiments, the amino acid sequence of VH CDR1 the amino acid sequence set forth in SEQ ID NO: 59 comprising no more than 1, 2, or 3 amino acid variations (e.g., substitution, deletion, addition, etc.); the amino acid sequence of VH CDR2 comprises the amino acid sequence set forth in SEQ ID NO: 60 comprising no more than 1, 2, or 3 amino acid variations (e.g., substitution, deletion, addition, etc.); the amino acid sequence of VH CDR3 comprises the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 59, or the amino acid sequence set forth in SEQ ID NO: 59 comprising 1, 2, or 3 amino acid variations (e.g., substitution, deletion, addition, etc.); the amino acid sequence of VH CDR2 consists of the amino acid sequence set forth in SEQ ID NO: 60, or the amino acid sequence set forth in SEQ ID NO: 60 comprising 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR1 consists of the amino acid sequence set forth in SEQ ID NO: 59, or the amino acid sequence set forth in SEQ ID NO: 59 comprising no more than 1, 2, or 3 amino acid variations (e.g., substitution, deletion, addition, etc.); the amino acid sequence of VH CDR2 consists of the amino acid sequence set forth in SEQ ID NO: 60, or the amino acid sequence set forth in SEQ ID NO: 60 comprising no more than 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 59; the amino acid sequence of VH CDR2 consists of the amino acid sequence set forth in SEQ ID NO: 60; the amino acid sequence of VH CDR3 consists of the amino acid sequence set forth in SEQ ID NO: 5; the amino acid sequence of VL CDR1 consists of the amino acid sequence set forth in SEQ ID NO: 6; the amino acid sequence of VL CDR2 consists of the amino acid sequence set forth in SEQ ID NO: 7; and the amino acid sequence of VL CDR3 consists of the amino acid sequence set forth in SEQ ID NO: 8.

In some embodiments, the amino acid sequence of VH CDR1 consists of the amino acid sequence set forth in SEQ ID NO: 59 comprising no more than 1, 2, or 3 amino acid variations (e.g., substitution, deletion, addition, etc.); the amino acid sequence of VH CDR2 consists of the amino acid sequence set forth in SEQ ID NO: 60 comprising no more than 1, 2, or 3 amino acid variations (e.g., substitution, deletion, addition, etc.); the amino acid sequence of VH CDR3 consists of the amino acid sequence set forth in SEQ ID NO: 5 comprising no more than 1, 2, or 3 amino acid variations (e.g., substitution, deletion, addition, etc.); the amino acid sequence of VL CDR1 consists of the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8 comprising no more than 1, 2, or 3 amino acid variations (e.g., substitution, deletion, addition, etc.).

In some embodiments, the amino acid sequence of VH CDR 1 comprises the amino acid sequence set forth in SEQ ID NO: 61, or the amino acid sequence set forth in SEQ ID NO: 61 comprising 1, 2, or 3 amino acid variations (e.g., substitution, deletion, addition, etc.); the amino acid sequence of VH CDR2 comprises the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR1 comprises the amino acid sequence set forth in SEQ ID NO: 61, or the amino acid sequence set forth in SEQ ID NO: 61 comprising no more than 1, 2, or 3 amino acid variations (e.g., substitution, deletion, addition, etc.); the amino acid sequence of VH CDR2 comprises the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of VH CDR3 comprises the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 comprises the amino acid sequence set forth in SEQ ID NO: 61; the amino acid sequence of VH CDR2 comprises the amino acid sequence set forth in SEQ ID NO: 62; the amino acid sequence of VH CDR3 comprises the amino acid sequence set forth in SEQ ID NO: 5; the amino acid sequence of VL CDR1 comprises the amino acid sequence set forth in SEQ ID NO: 6; the amino acid sequence of VL CDR2 comprises the amino acid sequence set forth in SEQ ID NO: 7; and the amino acid sequence of VL CDR3 comprises the amino acid sequence set forth in SEQ ID NO: 8.

In some embodiments, the amino acid sequence of VH CDR1 the amino acid sequence set forth in SEQ ID NO: 61 comprising no more than 1, 2, or 3 amino acid variations (e.g., substitution, deletion, addition, etc.); the amino acid sequence of VH CDR2 comprises the amino acid sequence set forth in SEQ ID NO: 62 comprising no more than 1, 2, or 3 amino acid variations (e.g., substitution, deletion, addition, etc.); the amino acid sequence of VH CDR3 comprises the amino acid sequence set forth in SEQ ID NO: 5 comprising no more than 1, 2, or 3 amino acid variations (e.g., substitution, deletion, addition, etc.); the amino acid sequence of VL CDR1 comprises the amino acid sequence set forth in SEQ ID NO: 6 comprising no more than 1, 2, or 3 amino acid variations (e.g., substitution, deletion, addition, etc.); the amino acid sequence of VL CDR2 comprises the amino acid sequence set forth in SEQ ID NO: 7 comprising no more than 1, 2, or 3 amino acid variations (e.g., substitution, deletion, addition, etc.); and the amino acid sequence of VL CDR3 comprises the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 61, or the amino acid sequence set forth in SEQ ID NO: 61 comprising 1, 2, or 3 amino acid variations (e.g., substitution, deletion, addition, etc.); the amino acid sequence of VH CDR2 consists of the amino acid sequence set forth in SEQ ID NO: 62, or the amino acid sequence set forth in SEQ ID NO: 62 comprising 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR1 consists of the amino acid sequence set forth in SEQ ID NO: 61, or the amino acid sequence set forth in SEQ ID NO: 61 comprising no more than 1, 2, or 3 amino acid variations (e.g., substitution, deletion, addition, etc.); the amino acid sequence of VH CDR2 consists of the amino acid sequence set forth in SEQ ID NO: 62, or the amino acid sequence set forth in SEQ ID NO: 62 comprising no more than 1, 2, or 3 amino acid variations (e.g., substitution, deletion, addition, etc.); the amino acid sequence of VH CDR3 consists of the amino acid sequence set forth in SEQ ID NO: 5, or the amino acid sequence set forth in SEQ ID NO: 5 comprising no more than 1, 2, or 3 amino acid variations (e.g., substitution, deletion, addition, etc.); the amino acid sequence of VL CDR1 consists of the amino acid sequence set forth in SEQ ID NO: 6, or the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7, or the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 61; the amino acid sequence of VH CDR2 consists of the amino acid sequence set forth in SEQ ID NO: 62; the amino acid sequence of VH CDR3 consists of the amino acid sequence set forth in SEQ ID NO: 5; the amino acid sequence of VL CDR1 consists of the amino acid sequence set forth in SEQ ID NO: 6; the amino acid sequence of VL CDR2 consists of the amino acid sequence set forth in SEQ ID NO: 7; and the amino acid sequence of VL CDR3 consists of the amino acid sequence set forth in SEQ ID NO: 8.

In some embodiments, the amino acid sequence of VH CDR1 consists of the amino acid sequence set forth in SEQ ID NO: 61 comprising no more than 1, 2, or 3 amino acid variations (e.g., substitution, deletion, addition, etc.); the amino acid sequence of VH CDR2 consists of the amino acid sequence set forth in SEQ ID NO: 62 comprising no more than 1, 2, or 3 amino acid variations (e.g., substitution, deletion, addition, etc.); the amino acid sequence of VH CDR3 consists of the amino acid sequence set forth in SEQ ID NO: 5 comprising no more than 1, 2, or 3 amino acid variations (e.g., substitution, deletion, addition, etc.); the amino acid sequence of VL CDR1 consists of the amino acid sequence set forth in SEQ ID NO: 6 comprising no more than 1, 2, or 3 amino acid variations (e.g., substitution, deletion, addition, etc.); the amino acid sequence of VL CDR2 consists of the amino acid sequence set forth in SEQ ID NO: 7 comprising no more than 1, 2, or 3 amino acid variations (e.g., substitution, deletion, addition, etc.); and the amino acid sequence of VL CDR3 consists of the amino acid sequence set forth in SEQ ID NO: 8 comprising no more than 1, 2, or 3 amino acid variations (e.g., substitution, deletion, addition, etc.).

In some embodiments, the amino acid sequence of VH CDR 1 comprises the amino acid sequence set forth in SEQ ID NO: 63, or the amino acid sequence set forth in SEQ ID NO: 63 comprising 1, 2, or 3 amino acid variations (e.g., substitution, deletion, addition, etc.); the amino acid sequence of VH CDR2 comprises the amino acid sequence set forth in SEQ ID NO: 64, or the amino acid sequence set forth in SEQ ID NO: 64 comprising 1, 2, or 3 amino acid variations (e.g., substitution, deletion, addition, etc.); the amino acid sequence of VH CDR3 comprises the amino acid sequence set forth in SEQ ID NO: 65, or the amino acid sequence set forth in SEQ ID NO: 65 comprising 1, 2, or 3 amino acid variations (e.g., substitution, deletion, addition, etc.); the amino acid sequence of VL CDR1 comprises the amino acid sequence set forth in SEQ ID NO: 66, or the amino acid sequence set forth in SEQ ID NO: 66 comprising 1, 2, or 3 amino acid variations (e.g., substitution, deletion, addition, etc.); the amino acid sequence of VL CDR2 comprises amino acid sequence GAS, or amino acid sequence amino acid sequence GAS comprising 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR 1 comprises the amino acid sequence set forth in SEQ ID NO: 63, or the amino acid sequence set forth in SEQ ID NO: 63 comprising no more than 1, 2, or 3 amino acid variations (e.g., substitution, deletion, addition, etc.); the amino acid sequence of VH CDR2 comprises the amino acid sequence set forth in SEQ ID NO: 64, or the amino acid sequence set forth in SEQ ID NO: 64 comprising no more than 1, 2, or 3 amino acid variations (e.g., substitution, deletion, addition, etc.); the amino acid sequence of VH CDR3 comprises the amino acid sequence set forth in SEQ ID NO: 65, or the amino acid sequence set forth in SEQ ID NO: 65 comprising no more than 1, 2, or 3 amino acid variations (e.g., substitution, deletion, addition, etc.); the amino acid sequence of VL CDR1 comprises the amino acid sequence set forth in SEQ ID NO: 66, or the amino acid sequence set forth in SEQ ID NO: 66 comprising no more than 1, 2, or 3 amino acid variations (e.g., substitution, deletion, addition, etc.); the amino acid sequence of VL CDR2 comprises amino acid sequence GAS, or amino acid sequence GAS comprising no more than 1, 2, or 3 amino acid variations (e.g., substitution, deletion, addition, etc.); and the amino acid sequence of VL CDR3 comprises the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR 1 comprises the amino acid sequence set forth in SEQ ID NO: 63; the amino acid sequence of VH CDR2 comprises the amino acid sequence set forth in SEQ ID NO: 64; the amino acid sequence of VH CDR3 comprises the amino acid sequence set forth in SEQ ID NO: 65; the amino acid sequence of VL CDR1 comprises the amino acid sequence set forth in SEQ ID NO: 66; the amino acid sequence of VL CDR2 comprises amino acid sequence GAS; and the amino acid sequence of VL CDR3 comprises the amino acid sequence set forth in SEQ ID NO: 8.

In some embodiments, the amino acid sequence of VH CDR1 the amino acid sequence set forth in SEQ ID NO: 63 comprising no more than 1, 2, or 3 amino acid variations (e.g., substitution, deletion, addition, etc.); the amino acid sequence of VH CDR2 comprises the amino acid sequence set forth in SEQ ID NO: 64 comprising no more than 1, 2, or 3 amino acid variations (e.g., substitution, deletion, addition, etc.); the amino acid sequence of VH CDR3 comprises the amino acid sequence set forth in SEQ ID NO: 65 comprising no more than 1, 2, or 3 amino acid variations (e.g., substitution, deletion, addition, etc.); the amino acid sequence of VL CDR1 comprises the amino acid sequence set forth in SEQ ID NO: 66 comprising no more than 1, 2, or 3 amino acid variations (e.g., substitution, deletion, addition, etc.); the amino acid sequence of VL CDR2 comprises amino acid sequence GAS comprising no more than 1, 2, or 3 amino acid variations (e.g., substitution, deletion, addition, etc.); and the amino acid sequence of VL CDR3 comprises the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 63, or the amino acid sequence set forth in SEQ ID NO: 63 comprising 1, 2, or 3 amino acid variations (e.g., substitution, deletion, addition, etc.); the amino acid sequence of VH CDR2 consists of the amino acid sequence set forth in SEQ ID NO: 64, or the amino acid sequence set forth in SEQ ID NO: 64 comprising 1, 2, or 3 amino acid variations (e.g., substitution, deletion, addition, etc.); the amino acid sequence of VH CDR3 consists of the amino acid sequence set forth in SEQ ID NO: 65, or the amino acid sequence set forth in SEQ ID NO: 65 comprising 1, 2, or 3 amino acid variations (e.g., substitution, deletion, addition, etc.); the amino acid sequence of VL CDR1 consists of the amino acid sequence set forth in SEQ ID NO: 66, or the amino acid sequence set forth in SEQ ID NO: 66 comprising 1, 2, or 3 amino acid variations (e.g., substitution, deletion, addition, etc.); the amino acid sequence of VL CDR2 consists of amino acid sequence GAS, or amino acid sequence GAS comprising 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising 1, 2, or 3 amino acid variations (e.g., substitution, deletion, addition, etc.).

In some embodiments, the amino acid sequence of VH CDR1 consists of the amino acid sequence set forth in SEQ ID NO: 63, or the amino acid sequence set forth in SEQ ID NO: 63 comprising no more than 1, 2, or 3 amino acid variations (e.g., substitution, deletion, addition, etc.); the amino acid sequence of VH CDR2 consists of the amino acid sequence set forth in SEQ ID NO: 64, or the amino acid sequence set forth in SEQ ID NO: 64 comprising no more than 1, 2, or 3 amino acid variations (e.g., substitution, deletion, addition, etc.); the amino acid sequence of VH CDR3 consists of the amino acid sequence set forth in SEQ ID NO: 65, or the amino acid sequence set forth in SEQ ID NO: 65 comprising no more than 1, 2, or 3 amino acid variations (e.g., substitution, deletion, addition, etc.); the amino acid sequence of VL CDR1 consists of the amino acid sequence set forth in SEQ ID NO: 66, or the amino acid sequence set forth in SEQ ID NO: 66 comprising no more than 1, 2, or 3 amino acid variations (e.g., substitution, deletion, addition, etc.); the amino acid sequence of VL CDR2 consists of amino acid sequence GAS, or amino acid sequence GAS comprising no more than 1, 2, or 3 amino acid variations (e.g., substitution, deletion, addition, etc.); and the amino acid sequence of VL CDR3 consists of the amino acid sequence set forth in SEQ ID NO: 8, or the amino acid sequence set forth in SEQ ID NO: 8 comprising no more than 1, 2, or 3 amino acid variations (e.g., substitution, deletion, addition, etc.).

In some embodiments, the amino acid sequence of VH CDR1 consists of the amino acid sequence set forth in SEQ ID NO: 63; the amino acid sequence of VH CDR2 consists of the amino acid sequence set forth in SEQ ID NO: 64; the amino acid sequence of VH CDR3 consists of the amino acid sequence set forth in SEQ ID NO: 65; the amino acid sequence of VL CDR1 consists of the amino acid sequence set forth in SEQ ID NO: 66; the amino acid sequence of VL CDR2 consists of amino acid sequence GAS; and the amino acid sequence of VL CDR3 consists of the amino acid sequence set forth in SEQ ID NO: 8.

In some embodiments, the amino acid sequence of VH CDR1 consists of the amino acid sequence set forth in SEQ ID NO: 63 comprising no more than 1, 2, or 3 amino acid variations (e.g., substitution, deletion, addition, etc.); the amino acid sequence of VH CDR2 consists of the amino acid sequence set forth in SEQ ID NO: 64 comprising no more than 1, 2, or 3 amino acid variations (e.g., substitution, deletion, addition, etc.); the amino acid sequence of VH CDR3 consists of the amino acid sequence set forth in SEQ ID NO: 65 comprising no more than 1, 2, or 3 amino acid variations (e.g., substitution, deletion, addition, etc.); the amino acid sequence of VL CDR1 consists of the amino acid sequence set forth in SEQ ID NO: 66 comprising no more than 1, 2, or 3 amino acid variations (e.g., substitution, deletion, addition, etc.); the amino acid sequence of VL CDR2 consists of amino acid sequence GAS comprising no more than 1, 2, or 3 amino acid variations (e.g., substitution, deletion, addition, etc.); and the amino acid sequence of VL CDR3 consists of the amino acid sequence set forth in SEQ ID NO: 8 comprising no more than 1, 2, or 3 amino acid variations (e.g., substitution, deletion, addition, etc.).

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 9; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH comprises an amino acid sequence at least 95%, 96%, 97%, 98%, 99%, or 100% identical to the amino acid sequence set forth in SEQ ID NO: 9; and the amino acid sequence of the VL comprises an amino acid sequence at least 95%, 96%, 97%, 98%, 99%, or 100% identical to the amino acid sequence set forth in SEQ ID NO: 10. In some embodiments, the amino acid sequence of the VH comprises the amino acid sequence set forth in SEQ ID NO: 9; and the amino acid sequence of the VL comprises the amino acid sequence set forth in SEQ ID NO: 10.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH consists of an amino acid sequence at least 90%, 91%, 92%, 93%, 94%, 95%, 96%, 97%, 98%, 99%, or 100% identical to the amino acid sequence set forth in SEQ ID NO: 9; and the amino acid sequence of the VL consists of an amino acid sequence at least 90%, 91%, 92%, 93%, 94%, 95%, 96%, 97%, 98%, 99%, or 100% identical to the amino acid sequence set forth in SEQ ID NO: 10. In some embodiments, the amino acid sequence of the VH consists of an amino acid sequence at least 95%, 96%, 97%, 98%, 99%, or 100% identical to the amino acid sequence set forth in SEQ ID NO: 9; and the amino acid sequence of the VL consists of an amino acid sequence at least 95%, 96%, 97%, 98%, 99%, or 100% identical to the amino acid sequence set forth in SEQ ID NO: 10. In some embodiments, the amino acid sequence of the VH consists of the amino acid sequence set forth in SEQ ID NO: 9; and the amino acid sequence of the VL consists of the amino acid sequence set forth in SEQ ID NO: 10.

In some embodiments, the CD161 binding protein comprises a light chain (LC) and a heavy chain (HC).

In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67; and the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68. In some embodiments, the amino acid sequence of the HC comprises an amino acid sequence at least 90%, 91%, 92%, 93%, 94%, 95%, 96%, 97%, 98%, 99%, or 100% identical to the amino acid sequence set forth in SEQ ID NO: 67; and the amino acid sequence of the LC comprises an amino acid sequence at least 90%, 91%, 92%, 93%, 94%, 95%, 96%, 97%, 98%, 99%, or 100% identical to the amino acid sequence set forth in SEQ ID NO: 68. In some embodiments, the amino acid sequence of the HC comprises an amino acid sequence at least 95%, 96%, 97%, 98%, 99%, or 100% identical to the amino acid sequence set forth in SEQ ID NO: 67; and the amino acid sequence of the LC comprises an amino acid sequence at least 95%, 96%, 97%, 98%, 99%, or 100% identical to the amino acid sequence set forth in SEQ ID NO: 68. In some embodiments, the amino acid sequence of the HC comprises the amino acid sequence set forth in SEQ ID NO: 67; and the amino acid sequence of the LC comprises the amino acid sequence set forth in SEQ ID NO: 68.

In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67; and the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68. In some embodiments, the amino acid sequence of the HC consists of an amino acid sequence at least 90%, 91%, 92%, 93%, 94%, 95%, 96%, 97%, 98%, 99%, or 100% identical to the amino acid sequence set forth in SEQ ID NO: 67; and the amino acid sequence of the LC consists of an amino acid sequence at least 90%, 91%, 92%, 93%, 94%, 95%, 96%, 97%, 98%, 99%, or 100% identical to the amino acid sequence set forth in SEQ ID NO: 68. In some embodiments, the amino acid sequence of the HC consists of an amino acid sequence at least 95%, 96%, 97%, 98%, 99%, or 100% identical to the amino acid sequence set forth in SEQ ID NO: 67; and the amino acid sequence of the LC consists of an amino acid sequence at least 95%, 96%, 97%, 98%, 99%, or 100% identical to the amino acid sequence set forth in SEQ ID NO: 68. In some embodiments, the amino acid sequence of the HC consists of the amino acid sequence set forth in SEQ ID NO: 67; and the amino acid sequence of the LC consists of the amino acid sequence set forth in SEQ ID NO: 68.

### 5.3 Ig Constant Regions

In some embodiments, a CD161 binding protein described herein (e.g., an anti-CD161 antibody (e.g., described herein)) comprises one or more Ig (e.g., human Ig (hIg)) constant region (e.g., a CH1 region, a hinge region, a CH2 region, a CH3 region, an Fc region, λ CL, κ CL).

In some embodiments, the CD161 binding protein (e.g., described herein) (e.g., an anti-CD161 antibody (e.g., described herein)) comprises one or more Ig (e.g., hIg) heavy chain constant region (e.g., a CH1 region, a hinge region, a CH2 region, a CH3 region, an Fc region). For example, a full-length antibody CD161 binding protein comprises a CH1 region, a hinge region, a CH2 region, and a CH3 region. Or for example, the CD161 binding protein may be fused to one or more Ig (e.g., hIg) heavy chain constant regions (e.g., a CH1 region, a hinge region, a CH2 region, a CH3 region, an Fc region).

In some embodiments, the Ig is a hIg. In some embodiments, the Ig is a human IgG (IgG). In some embodiments, the hIgG is hIgG1, hIgG2 (e.g., hIgG2a or hIgG2b), hIgG3, or hIgG4. In some embodiments, the hIgG is hIgG1 or hIgG4. In some embodiments, the hIgG is hIgG1. In some embodiments, the hIgG is hIgG4.

In some embodiments, the CD161 binding protein (e.g., described herein) (e.g., an anti-CD161 antibody (e.g., described herein)) comprises an Fc region. In some embodiments, the Fc region is part of a full-length antibody. In some embodiments, the Fc region comprises or consists of at least a portion of a hinge region, a CH2 region, and a CH3 region. In some embodiments, the Fc region comprises or consists of a hinge region, a CH2 region, and a CH3 region. In some embodiments, the Fc region comprises or consists of at least a portion of an IgG hinge region, an IgG CH2 region, and an IgG CH3 region. In some embodiments, the Fc region comprises or consists of an IgG hinge region, an IgG CH2 region, and an IgG CH3 region. In some embodiments, the Fc region comprises or consists of at least a portion of an IgG1 hinge region, an IgG1 CH2 region, and an IgG1 CH3 region. In some embodiments, the Fc region comprises or consists of an IgG1 hinge region, an IgG1 CH2 region, and an IgG1 CH3 region. In some embodiments, the Fc region comprises or consists of at least a portion of an IgG4 hinge region, an IgG4 CH2 region, and an IgG4 CH3 region. In some embodiments, the Fc region comprises or consists of an IgG4 hinge region, an IgG4 CH2 region, and an IgG4 CH3 region. In some embodiments, the Fc region comprises or consists of at least a portion of an hIgG hinge region, a hIgG CH2 region, and an hIgG CH3 region. In some embodiments, the Fc region comprises or consists of a hIgG hinge region, an hIgG CH2 region, and a hIgG CH3 region. In some embodiments, the Fc region comprises or consists of at least a portion of an hIgG1 hinge region, an hIgG1 CH2 region, and an hIgG1 CH3 region. In some embodiments, the Fc region comprises or consists of an hIgG1 hinge region, an hIgG1 CH2 region, and an hIgG1 CH3 region. In some embodiments, the Fc region comprises or consists of at least a portion of a hIgG4 hinge region, an hIgG4 CH2 region, and an hIgG4 CH3 region. In some embodiments, the Fc region comprises or consists of a hIgG4 hinge region, an hIgG4 CH2 region, and a hIgG4 CH3 region.

In some embodiments, the CD161 binding protein (e.g., described herein) (e.g., an anti-CD161 antibody (e.g., described herein)) comprises one or more Ig (e.g., hIg) light chain constant regions (e.g., ΔCL, KCL). In some embodiments, the one or more Ig light chain constant regions are hIg. For example, a full-length antibody comprises two light chains each comprising a light chain constant region (e.g., ACL, KCL).

The amino acid sequence of exemplary reference IgG1 and IgG4 heavy chain constant regions and Ig light chain constant regions, which can be incorporated in one or more of the embodiments described herein (e.g., CD161 binding proteins, conjugates, and fusion proteins), is provided in Table 3.

TABLE 3
The Amino Acid Sequence of Exemplary hIg heavy and light chain constant
regions & components.

| | | SEQ |
|---|---|---|
| Description | Amino Acid Sequence | ID NO |
| IgG1 CH1 Region | ASTKGPSVFPLAPSSKSTSGGTAALGCLVKDYFPEPVTV | 11 |
| | SWNSGALTSGVHTFPAVLOSSGLYSLSSVVTVPSSSLGT | |
| | QTYICNVNHKPSNTKVDKKV | |
| IgG1 Hinge Region | EPKSCDKTHTCP | 12 |
| IgG1 CH2 Region | PCPAPELLGGPSVFLFPPKPKDTLMISRTPEVTCVVVDV | 13 |
| | SHEDPEVKFNWYVDGVEVHNAKTKPREEQYNSTYRVVSV | |
| | LTVLHQDWLNGKEYKCKVSNKALPAPIEKTISKAK | |
| IgG1 CH3 Region | GQPREPQVYTLPPSRDELTKNQVSLTCLVKGFYPSDIAV | 14 |
| With C-terminal Lysine | EWESNGQPENNYKTTPPVLDSDGSFFLYSKLTVDKSRWQ | |
| | QGNVFSCSVMHEALHNHYTQKSLSLSPGK | |
| IgG1 CH3 Region | GQPREPQVYTLPPSRDELTKNQVSLTCLVKGFYPSDIAV | 15 |
| Without C-terminal | EWESNGQPENNYKTTPPVLDSDGSFFLYSKLTVDKSRWQ | |
| Lysine | QGNVFSCSVMHEALHNHYTQKSLSLSPG | |
| IgG1 CH2 Region + | PCPAPELLGGPSVFLFPPKPKDTLMISRTPEVTCVVVDV | 16 |
| CH3 Region | SHEDPEVKFNWYVDGVEVHNAKTKPREEQYNSTYRVVSV | |
| With C-terminal Lysine | LTVLHQDWLNGKEYKCKVSNKALPAPIEKTISKAKGQPR | |
| | EPQVYTLPPSRDELTKNQVSLTCLVKGFYPSDIAVEWES | |
| | NGQPENNYKTTPPVLDSDGSFFLYSKLTVDKSRWQQGNV | |
| | FSCSVMHEALHNHYTQKSLSLSPGK | |
| IgG1 CH2 Region + | PCPAPELLGGPSVFLFPPKPKDTLMISRTPEVTCVVVDV | 17 |
| CH3 Region | SHEDPEVKFNWYVDGVEVHNAKTKPREEQYNSTYRVVSV | |
| Without C-terminal | LTVLHQDWLNGKEYKCKVSNKALPAPIEKTISKAKGQPR | |
| Lysine | EPQVYTLPPSRDELTKNQVSLTCLVKGFYPSDIAVEWES | |
| | NGQPENNYKTTPPVLDSDGSFFLYSKLTVDKSRWQQGNV | |
| | FSCSVMHEALHNHYTQKSLSLSPG | |
| IgG1 Partial Hinge | TCPPCPAPELLGGPSVFLFPPKPKDTLMISRTPEVTCVV | 18 |
| Region + CH2 Region + | VDVSHEDPEVKFNWYVDGVEVHNAKTKPREEQYNSTYRV | |
| CH3 Region | VSVLTVLHQDWLNGKEYKCKVSNKALPAPIEKTISKAKG | |
| With C-terminal Lysine | QPREPQVYTLPPSRDELTKNQVSLTCLVKGFYPSDIAVE | |
| | WESNGQPENNYKTTPPVLDSDGSFFLYSKLTVDKSRWQQ | |
| | GNVFSCSVMHEALHNHYTQKSLSLSPGK | |
| IgG1 Partial Hinge | TCPPCPAPELLGGPSVFLFPPKPKDTLMISRTPEVTCVV | 19 |
| Region + CH2 Region + | VDVSHEDPEVKFNWYVDGVEVHNAKTKPREEQYNSTYRV | |
| CH3 Region | VSVLTVLHQDWLNGKEYKCKVSNKALPAPIEKTISKAKG | |
| Without C-terminal | QPREPQVYTLPPSRDELTKNQVSLTCLVKGFYPSDIAVE | |
| Lysine | WESNGQPENNYKTTPPVLDSDGSFFLYSKLTVDKSRWQQ | |
| | GNVFSCSVMHEALHNHYTQKSLSLSPG | |
| IgG1 Hinge Region + | EPKSCDKTHTCPPCPAPELLGGPSVFLFPPKPKDTLMIS | 20 |
| CH2 Region + CH3 | RTPEVTCVVVDVSHEDPEVKFNWYVDGVEVHNAKTKPRE | |
| Region | EQYNSTYRVVSVLTVLHQDWLNGKEYKCKVSNKALPAPI | |
| With C-terminal Lysine | EKTISKAKGQPREPQVYTLPPSRDELTKNQVSLTCLVKG | |
| | FYPSDIAVEWESNGQPENNYKTTPPVLDSDGSFFLYSKL | |
| | TVDKSRWQQGNVFSCSVMHEALHNHYTQKSLSLSPGK | |
| IgG1 Hinge Region + | EPKSCDKTHTCPPCPAPELLGGPSVFLFPPKPKDTLMIS | 21 |
| CH2 Region + CH3 | RTPEVTCVVVDVSHEDPEVKFNWYVDGVEVHNAKTKPRE | |
| Region | EQYNSTYRVVSVLTVLHQDWLNGKEYKCKVSNKALPAPI | |
| Without C-terminal | EKTISKAKGQPREPQVYTLPPSRDELTKNQVSLTCLVKG | |
| Lysine | FYPSDIAVEWESNGQPENNYKTTPPVLDSDGSFFLYSKL | |
| | TVDKSRWQQGNVFSCSVMHEALHNHYTQKSLSLSPG | |
| IgG1 CH1+ Hinge | ASTKGPSVFPLAPSSKSTSGGTAALGCLVKDYFPEPVTV | 22 |
| Region + CH2 Region + | SWNSGALTSGVHTFPAVLQSSGLYSLSSVVTVPSSSLGT | |
| CH3 Region | QTYICNVNHKPSNTKVDKKVEPKSCDKTHTCPPCPAPEL | |
| With C-terminal Lysine | LGGPSVFLFPPKPKDTLMISRTPEVTCVVVDVSHEDPEV | |
| | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPGK | |
| IgG1 CH1 + Hinge | ASTKGPSVFPLAPSSKSTSGGTAALGCLVKDYFPEPVTV | 23 |
| Region + CH2 Region + | SWNSGALTSGVHTFPAVLQSSGLYSLSSVVTVPSSSLGT | |
| CH3 Region | QTYICNVNHKPSNTKVDKKVEPKSCDKTHTCPPCPAPEL | |
| Without C-terminal | LGGPSVFLFPPKPKDTLMISRTPEVTCVVVDVSHEDPEV | |
| Lysine | KFNWYVDGVEVHNAKTKPREEQYNSTYRVVSVLTVLHQD | |
| | WLNGKEYKCKVSNKALPAPIEKTISKAKGQPREPQVYTL | |
| | PPSRDELTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| IgG4 CH1 Region | ASTKGPSVFPLAPCSRSTSESTAALGCLVKDYFPEPVTV | 24 |
| | SWNSGALTSGVHTFPAVLOSSGLYSLSSVVTVPSSSLGT | |
| | KTYTCNVDHKPSNTKVDKRV | |
| IgG4 Hinge Region | ESKYGPPCPSCP | 25 |
| IgG4 CH2 Region | APEFLGGPSVFLFPPKPKDTLMISRTPEVTCVVVDVSQE | 26 |
| | DPEVQFNWYVDGVEVHNAKTKPREEQFNSTYRVVSVLTV | |
| | LHQDWLNGKEYKCKVSNKGLPSSIEKTISKAK | |
| IgG4 CH3 Region | GQPREPQVYTLPPSQEEMTKNQVSLTCLVKGFYPSDIAV | 27 |
| With C-terminal Lysine | EWESNGQPENNYKTTPPVLDSDGSFFLYSRLTVDKSRWQ | |
| | EGNVFSCSVMHEALHNHYTQKSLSLSLGK | |
| IgG4 CH3 Region | GQPREPQVYTLPPSQEEMTKNQVSLTCLVKGFYPSDIAV | 28 |
| Without C-terminal | EWESNGQPENNYKTTPPVLDSDGSFFLYSRLTVDKSRWQ | |
| Lysine | EGNVFSCSVMHEALHNHYTQKSLSLSLG | |
| IgG4 CH2 Region + | APEFLGGPSVFLFPPKPKDTLMISRTPEVTCVVVDVSQE | 29 |
| CH3 Region | DPEVQFNWYVDGVEVHNAKTKPREEQFNSTYRVVSVLTV | |
| With C-terminal Lysine | LHQDWLNGKEYKCKVSNKGLPSSIEKTISKAKGQPREPQ | |
| | VYTLPPSQEEMTKNQVSLTCLVKGFYPSDIAVEWESNGQ | |
| | PENNYKTTPPVLDSDGSFFLYSRLTVDKSRWQEGNVFSC | |
| | SVMHEALHNHYTQKSLSLSLGK | |
| IgG4 CH2 Region + | APEFLGGPSVFLFPPKPKDTLMISRTPEVTCVVVDVSQE | 30 |
| CH3 Region | DPEVQFNWYVDGVEVHNAKTKPREEQFNSTYRVVSVLTV | |
| Without C-terminal | LHQDWLNGKEYKCKVSNKGLPSSIEKTISKAKGQPREPQ | |
| Lysine | VYTLPPSQEEMTKNQVSLTCLVKGFYPSDIAVEWESNGQ | |
| | PENNYKTTPPVLDSDGSFFLYSRLTVDKSRWQEGNVFSC | |
| | SVMHEALHNHYTQKSLSLSLG | |
| IgG4 Partial Hinge | PCPSCPAPEFLGGPSVFLFPPKPKDTLMISRTPEVTCVV | 31 |
| Region + CH2 Region + | VDVSQEDPEVQFNWYVDGVEVHNAKTKPREEQFNSTYRV | |
| CH3 Region | VSVLTVLHQDWLNGKEYKCKVSNKGLPSSIEKTISKAKG | |
| With C-terminal Lysine | QPREPQVYTLPPSQEEMTKNQVSLTCLVKGFYPSDIAVE | |
| | WESNGQPENNYKTTPPVLDSDGSFFLYSRLTVDKSRWQE | |
| | GNVFSCSVMHEALHNHYTQKSLSLSLGK | |
| IgG4 Partial Hinge | PCPSCPAPEFLGGPSVFLFPPKPKDTLMISRTPEVTCVV | 32 |
| Region + CH2 Region + | VDVSQEDPEVQFNWYVDGVEVHNAKTKPREEQFNSTYRV | |
| CH3 Region | VSVLTVLHQDWLNGKEYKCKVSNKGLPSSIEKTISKAKG | |
| Without C-terminal | QPREPQVYTLPPSQEEMTKNQVSLTCLVKGFYPSDIAVE | |
| Lysine | WESNGQPENNYKTTPPVLDSDGSFFLYSRLTVDKSRWQE | |
| | GNVFSCSVMHEALHNHYTQKSLSLSLG | |
| IgG4 Hinge Region + | ESKYGPPCPSCPAPEFLGGPSVFLFPPKPKDTLMISRTP | 33 |
| CH2 Region + CH3 | EVTCVVVDVSQEDPEVQFNWYVDGVEVHNAKTKPREEQF | |
| Region | NSTYRVVSVLTVLHQDWLNGKEYKCKVSNKGLPSSIEKT | |
| With C-terminal Lysine | ISKAKGQPREPQVYTLPPSQEEMTKNQVSLTCLVKGFYP | |
| | SDIAVEWESNGQPENNYKTTPPVLDSDGSFFLYSRLTVD | |
| | KSRWQEGNVFSCSVMHEALHNHYTQKSLSLSLGK | |
| IgG4 Hinge Region + | ESKYGPPCPSCPAPEFLGGPSVFLFPPKPKDTLMISRTP | 34 |
| CH2 Region + CH3 | EVTCVVVDVSQEDPEVQFNWYVDGVEVHNAKTKPREEQF | |
| Region | NSTYRVVSVLTVLHQDWLNGKEYKCKVSNKGLPSSIEKT | |
| Without C-terminal | ISKAKGQPREPQVYTLPPSQEEMTKNQVSLTCLVKGFYP | |
| Lysine | SDIAVEWESNGQPENNYKTTPPVLDSDGSFFLYSRLTVD | |
| | KSRWQEGNVFSCSVMHEALHNHYTQKSLSLSLG | |
| IgG4 CH1 + Hinge | ASTKGPSVFPLAPCSRSTSESTAALGCLVKDYFPEPVTV | 35 |
| Region + CH2 Region + | SWNSGALTSGVHTFPAVLOSSGLYSLSSVVTVPSSSLGT | |
| CH3 Region | KTYTCNVDHKPSNTKVDKRVESKYGPPCPSCPAPEFLGG | |
| With C-terminal Lysine | PSVFLFPPKPKDTLMISRTPEVTCVVVDVSQEDPEVQFN | |
| | WYVDGVEVHNAKTKPREEQFNSTYRVVSVLTVLHQDWLN | |
| | GKEYKCKVSNKGLPSSIEKTISKAKGOPREPQVYTLPPS | |
| | QEEMTKNQVSLTCLVKGFYPSDIAVEWESNGQPENNYKT | |
| | TPPVLDSDGSFFLYSRLTVDKSRWQEGNVFSCSVMHEAL | |
| | HNHYTQKSLSLSLGK | |
| IgG4 CH1 + Hinge | ASTKGPSVFPLAPCSRSTSESTAALGCLVKDYFPEPVTV | 36 |
| Region + CH2 Region + | SWNSGALTSGVHTFPAVLQSSGLYSLSSVVTVPSSSLGT | |
| CH3 Region | KTYTCNVDHKPSNTKVDKRVESKYGPPCPSCPAPEFLGG | |
| Without C-terminal | PSVFLFPPKPKDTLMISRTPEVTCVVVDVSQEDPEVQFN | |
| Lysine | WYVDGVEVHNAKTKPREEQFNSTYRVVSVLTVLHQDWLN | |
| | GKEYKCKVSNKGLPSSIEKTISKAKGQPREPQVYTLPPS | |
| | QEEMTKNQVSLTCLVKGFYPSDIAVEWESNGQPENNYKT | |
| | TPPVLDSDGSFFLYSRLTVDKSRWQEGNVFSCSVMHEAL | |
| | HNHYTQKSLSLSLG | |
| Ig light chain kappa | RTVAAPSVFIFPPSDEQLKSGTASVVCLLNNFYPREAKV | 37 |
| constant region (KCL) | QWKVDNALQSGNSQESVTEQDSKDSTYSLSSTLTLSKAD | |
| | YEKHKVYACEVTHQGLSSPVTKSFNRGEC | |
| Ig light chain kappa | GQPKANPTVTLFPPSSEELQANKATLVCLISDFYPGAVT | 38 |
| constant region (ACL) | VAWKADGSPVKAGVETTKPSKQSNNKYAASSYLSLTPEQ | |
| | WKSHRSYSCQVTHEGSTVEKTVAPTECS | |

In some embodiments, the CD161 binding protein (e.g., described herein) (e.g., an anti-CD161 antibody (e.g., described herein)) comprises an Ig constant region comprising an amino acid sequence set forth in Table 3. In some embodiments, the CD161 binding protein (e.g., described herein) (e.g., an anti-CD161 antibody (e.g., described herein)) comprises an Ig constant region comprising an amino acid sequence set forth in Table 3, and further comprises 1 or more but less than 15% (less than 12%, less than 10%, less than 8%), amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising an amino acid sequence set forth in Table 3, comprising or consisting of at least about 1, 2, 3, 4, 5, 6, 7, 8, 9, 10, or more amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising an amino acid sequence set forth in Table 3, comprising or consisting of about 1, 2, 3, 4, 5, 6, 7, 8, 9, 10, or more amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising an amino acid sequence set forth in Table 3, comprising or consisting of about no more than 1, 2, 3, 4, 5, 6, 7, 8, 9, 10, or more amino acid variations (e.g., amino acid substitutions, deletions, or additions).

In some embodiments, the CD161 binding protein (e.g., described herein) (e.g., an anti-CD161 antibody (e.g., described herein)) comprises an Ig constant region comprising an amino acid sequence set forth in Table 3, and further comprises 1 or more but less than 15% (less than 12%, less than 10%, less than 8%), amino acid substitutions. In some embodiments, the CD161 binding protein (e.g., described herein) (e.g., an anti-CD161 antibody (e.g., described herein)) comprises an Ig constant region comprising an amino acid sequence set forth in Table 3, comprising or consisting of at least about 1, 2, 3, 4, 5, 6, 7, 8, 9, 10, or more amino acid substitutions. In some embodiments, the CD161 binding protein (e.g., described herein) (e.g., an anti-CD161 antibody (e.g., described herein)) comprises an Ig constant region comprising an amino acid sequence set forth in Table 3, comprising or consisting of about 1, 2, 3, 4, 5, 6, 7, 8, 9, 10, or more amino acid substitutions. In some embodiments, the CD161 binding protein (e.g., described herein) (e.g., an anti-CD161 antibody (e.g., described herein)) comprises an Ig constant region comprising an amino acid sequence set forth in Table 3, comprising or consisting of about no more than about 1, 2, 3, 4, 5, 6, 7, 8, 9, 10, or more amino acid substitutions.

In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38. In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, and further comprises 1 or more but less than 15% (less than 12%, less than 10%, less than 8%), amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising at least about 1, 2, 3, 4, 5, 6, 7, 8, 9, 10, or more amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising or consisting about 1, 2, 3, 4, 5, 6, 7, 8, 9, or 10 amino acid variations (e.g., amino acid substitutions, deletions, or additions). In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising or consisting of no more than about 1, 2, 3, 4, 5, 6, 7, 8, 9, or 10 amino acid variations (e.g., amino acid substitutions, deletions, or additions).

In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, and further comprises 1 or more but less than 15% (less than 12%, less than 10%, less than 8%), amino acid substitutions. In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38. In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising or consisting at least about 1, 2, 3, 4, 5, 6, 7, 8, 9, 10, or more amino acid substitutions. In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising or consisting about 1, 2, 3, 4, 5, 6, 7, 8, 9, or 10 amino acid substitutions. In some embodiments, the CD161 binding protein (e.g., described herein) (e.g., an anti-CD161 antibody (e.g., described herein)) comprises an Ig constant region comprising the amino acid sequence of any one or more of SEQ ID NOS: 11-38, comprising or consisting of no more than about 1, 2, 3, 4, 5, 6, 7, 8, 9, or 10 amino acid substitutions.

### 5.3.1 Ig Effector Function

In some embodiments, the CD161 binding protein (e.g., described herein) (e.g., an anti-CD161 antibody (e.g., described herein)) comprises an Ig (e.g., hIg) Fc region. In some embodiments, the Ig Fc region is part of a full-length antibody. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region. In some embodiments, the Ig Fc region comprises or consists of at least a portion of an IgG1 hinge region, an IgG1 CH2 region, and an IgG1 CH3 region. In some embodiments, the Ig Fc region comprises or consists of an IgG1 hinge region, an IgG1 CH2 region, and an IgG1 CH3 region. In some embodiments, the Ig Fc region comprises or consists of at least a portion of an IgG4 hinge region, an IgG4 CH2 region, and an IgG4 CH3 region. In some embodiments, the Ig Fc region comprises or consists of an IgG4 hinge region, an IgG4 CH2 region, and an IgG4 CH3 region.

In some embodiments, the CD161 binding protein (e.g., described herein) (e.g., an anti-CD161 antibody (e.g., described herein)) comprises a hIg Fc region. In some embodiments, the hIg Fc region is part of a full-length antibody. In some embodiments, the hIg Fc region comprises or consists of at least a portion of a hinge region, a CH2 region, and a CH3 region. In some embodiments, the hIg Fc region comprises or consists of a hinge region, a CH2 region, and a CH3 region. In some embodiments, the hIg Fc region comprises or consists of at least a portion of an hIgG hinge region, an hIgG CH2 region, and an hIgG CH3 region. In some embodiments, the hIg Fc region comprises or consists of an hIgG hinge region, an hIgG CH2 region, and an hIgG CH3 region. In some embodiments, the hIg Fc region comprises or consists of at least a portion of a hIgG1 hinge region, a hIgG1 CH2 region, and a hIgG1 CH3 region. In some embodiments, the Ig Fc region comprises or consists of a hIgG1 hinge region, a hIgG1 CH2 region, and a hIgG1 CH3 region. In some embodiments, the hIg Fc region comprises or consists of at least a portion of a hIgG4 hinge region, a hIgG4 CH2 region, and a hIgG4 CH3 region. In some embodiments, the hIg Fc region comprises or consists of a hIgG4 hinge region, a hIgG4 CH2 region, and a hIgG4 CH3 region.

In some embodiments, the Ig (e.g., hIg) Fc region exhibits an alteration (e.g., enhancement) in one or more Fc effector function relative to a reference (e.g., wild type) Ig Fc region. Exemplary Ig Fc effector functions include, but are not limited to, antibody dependent cellular cytotoxicity (ADCC), antibody dependent cellular phagocytosis (ADCP), complement dependent cytotoxicity (CDC), binding affinity to C1q, and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))).

Standard in vitro and/or in vivo assays known in the art can be conducted to evaluate Ig (e.g., hIg) Fc effector function, including, any one or more of ADCC, CDC, ADCP, Fc receptor (e.g., Fcγ receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (51Cr) assay. Briefly, 51Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide fluorophore (Europium). see, e.g., Parekh, Bhavin S et al. “Development and validation of an antibody-dependent cell-mediated cytotoxicity-reporter gene assay.” mAbs vol. 4, 3 (2012):310-8. Doi: 10.4161/mabs. 19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a CD161 binding protein (e.g., an anti-CD161 antibody) described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a CD161 binding protein (e.g., an anti-CD161 antibody) described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a CD161 binding protein (e.g., an anti-CD161 antibody) described herein to bind C1q (or bind with less affinity than a reference a CD161 binding protein (e.g., an anti-CD161 antibody)) and hence lack (or have decreased) CDC activity. The binding of a CD161 binding protein (e.g., an anti-CD161 antibody) described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4th Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a CD161 binding protein (e.g., an anti-CD161 antibody) described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi: 10.3791/3588; the entire contents of each of which is incorporated by reference herein.

Binding of an Ig (e.g., hIg) (e.g., an antibody comprising the foregoing) to an Ig Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated by reference herein for all purposes.

### 5.3.1.1 Enhanced Ig Effector Function

In some embodiments, the Ig Fc region of the CD161 binding protein (e.g., an anti-CD161 antibody) exhibits an enhancement (e.g., an increase) in one or more Fc effector function (e.g., relative to a reference (e.g., wild type) Ig Fc region). Exemplary Ig Fc effector functions include, but are not limited to, ADCC, ADCP, CDC, binding affinity to C1q, and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))).

In some embodiments, the Ig Fc region of the CD161 binding protein (e.g., an anti-CD161 antibody) is modified (e.g., comprises one or more variation (e.g., one or more amino acid substitution, deletion, addition, etc.); altered glycosylation (e.g., afucosylation))) (referred to herein as a “modified Ig Fc”). In some embodiments, the modification (e.g., the variation (e.g., one or more amino acid substitution, deletion, addition, etc.); altered glycosylation (e.g., afucosylation))) enhances (e.g., increases) one or more Fc effector function, relative to a reference Ig Fc that does not comprise the modification (e.g., the one or more variation (e.g., the one or more amino acid substitution, deletion, addition, etc.; the altered glycosylation (e.g., afucosylation))).

In some embodiments, the CD161 binding protein (e.g., an anti-CD161 antibody) comprises a modified Ig Fc and exhibits enhanced (e.g., increased) ADCC compared to a reference CD161 binding protein (e.g., an anti-CD161 antibody) that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein comprises a modified Ig Fc and exhibits enhanced (e.g., increased) CDC compared to a reference CD161 binding protein (e.g., an anti-CD161 antibody) that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein (e.g., an anti-CD161 antibody) comprises a modified Ig Fc and exhibits enhanced (e.g., increased) ADCP compared to a reference CD161 binding protein that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein (e.g., an anti-CD161 antibody) comprises a modified Ig Fc and exhibits enhanced (e.g., increased) binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))) compared to a reference CD161 binding protein that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein (e.g., an anti-CD161 antibody) comprises a modified Ig Fc and exhibits enhanced (e.g., increased) binding affinity to FcγRI, FcγRIIa, and/or FcγRIIIa compared to a reference CD161 binding protein that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein (e.g., an anti-CD161 antibody) comprises a modified Ig Fc and exhibits enhanced (e.g., increased) binding affinity to FcγRI compared to a reference CD161 binding protein that does not comprise the Ig Fc modification (e.g., the one or more variation (e.g., the one or more amino acid substitution, deletion, addition, etc.; the altered glycosylation (e.g., afucosylation))). In some embodiments, the a CD161 binding protein (e.g., an anti-CD161 antibody) comprises a modified Ig Fc and exhibits enhanced (e.g., increased) to FcγRIIa compared to a reference CD161 binding protein that does not comprise the Ig Fc modification (e.g., the one or more variation (e.g., the one or more amino acid substitution, deletion, addition, etc.; the altered glycosylation (e.g., afucosylation))). In some embodiments, the CD161 binding protein comprises a modified Ig Fc and exhibits enhanced (e.g., increased) binding affinity to FcγRIIIa compared to a reference CD161 binding protein that does not comprise the Ig Fc modification (e.g., the one or more variation (e.g., the one or more variation (e.g., the one or more amino acid substitution, deletion, addition, etc.; the altered glycosylation (e.g., afucosylation))). In some embodiments, the CD161 binding protein (e.g., an anti-CD161 antibody) comprises a modified Ig Fc and exhibits enhanced (e.g., increased) binding affinity to C1q compared to a reference CD161 binding protein that does not comprise the Ig Fc modification (e.g., the one or more variation (e.g., the one or more amino acid substitution, deletion, addition, etc.; the altered glycosylation (e.g., afucosylation)

Amino acid substitutions that enhance (e.g., increase) one or more Ig Fc effector function are known in the art. See for example, Liu R, Oldham R J, Teal E, Beers S A, Cragg M S. Fc-Engineering for Modulated Effector Functions-Improving Antibodies for Cancer Treatment. Antibodies (Basel). 2020; 9(4):64. Published 2020 Nov. 17. doi:10.3390/antib9040064; van der Horst H J, Nijhof I S, Mutis T, Chamuleau M E D. Fc-Engineered Antibodies with Enhanced Fc-Effector Function for the Treatment of B-Cell Malignancies. Cancers (Basel). 2020; 12(10):3041. Published 2020 Oct. 19. Doi: 10.3390/cancers12103041; and Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of each of which is incorporated by reference herein for all purposes.

Table 4 below, provides exemplary amino acid substitutions (and combinations thereof) and alterations in glycosylation (e.g., afucosylation) that can be utilized to increase one or more Ig Fc effector function. Amino acids in Table 4 are numbered according to the EU numbering scheme. The effects on effector function set forth in Table 4 are exemplary only and not intended to be limiting. The amino acid substitutions set forth in Table 4 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 4
Exemplary Ig Fc Variations and Glycoengineering
to Increase Effector Function.

| | Exemplary Effects on |
|---|---|
| Variation/Glycoengineering | Effector Function |

Amino Acid Substitutions

| S298A/E333A/K334A | | Increases ADCC |
|---|---|---|
| S239D/I332E | | Increases ADCC |
| P247I/A339Q | | Increases ADCC |
| S239D/A330L/I332E | | Increases ADCC |
| G236A/S239D/I332E | | Increases ADCC |
| F243L/R292P/Y300L/V305I/P396L | | Increases ADCC |
| L235V/F243L/R292P/Y300L/P396L | | Increases ADCC |
| One Heavy | Opposing Heavy | Increases ADCC |
| Chain: | Chain: | |
| L234Y/L235Q/ | D270E/K326D/ | |
| G236W/S239M/ | A330M/K334E | |
| H268D/D270E/ | | |
| S298A | | |

| | |
|---|---|
| F243L/R292P/Y300L/V305I/P396L | Increases ADCP |
| S239D/I332E/A330L | Increases ADCP |
| S239D/I332E/A330L/ G236A | Increases ADCP |
| S239D/I332E/G326A | Increases ADCP |
| G236A | Increases ADCP |
| G236A/S239D/I332E | Increases ADCP |
| S239D/I332E | Increases ADCP |
| K326W/E333S | Increases C1q Binding and CDC |
| S267E/H268E/S324T | Increases C1q Binding and CDC |
| S298A/E333A/K334A | Enhances FcγRIIIa binding |
| S239D/I332E | Enhances FcγRIIIa binding |
| P247I/A339Q | Enhances FcγRIIIa binding |
| F243L/R292P/Y300L/V305I/P396L | Enhances FcγRIIa binding; |
| | decreases FcβRIIb binding |
| G236A | Enhances FcγRIIa binding |

Glycoengineering

| | |
|---|---|
| Afucosylation | Increases ADCC, Increases ADCP |
| Galactosylation | Increases CDC |

In some embodiments, the Ig Fc (e.g., IgG1 Fc) region of a CD161 binding protein (e.g., an anti-CD161 antibody) comprises any one or more of the amino acid substitutions set forth in Table 4 (i.e., any one or more amino acid substitution set forth in any set of amino acid substitutions set forth in Table 4). In some embodiments, the Ig Fc (e.g., IgG1 Fc) region of a CD161 binding protein (e.g., an anti-CD161 antibody) comprises any one or more of the sets of amino acid substitutions set forth in Table 4. In some embodiments, the Ig Fc (e.g., IgG1 Fc) region of a CD161 binding protein (e.g., an anti-CD161 antibody) comprises any one or more of the glycosylation changes set forth in Table 4.

In some embodiments, the Ig Fc (e.g., IgG1 Fc) region of a CD161 binding protein (e.g., an anti-CD161 antibody) comprises an amino acid substitution at any one or more (e.g., 1, 2, 3, 4, 5, 6, 7, 8, 9, or 10, or more) of amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334. In some embodiments, the Ig Fc (e.g., IgG1 Fc) region of a CD161 binding protein (e.g., an anti-CD161 antibody) comprises an amino acid substitution at from about 1-10 (e.g., 1-9, 1-8, 1-7, 1-6, 1-5, 1-4, 1-3, or 1-2) of the following amino acid positions S298, E333, K334, S239, 1332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334.

In some embodiments, the Ig Fc (e.g., IgG1 Fc) region of a CD161 binding protein (e.g., an anti-CD161 antibody) comprises one or more (e.g., 1, 2, 3, 4, 5, 6, 7, 8, 9, or 10, or more) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the Ig Fc (e.g., IgG1 Fc) region of a CD161 binding protein (e.g., an anti-CD161 antibody) comprises from about 1-10 (e.g., 1-9, 1-8, 1-7, 1-6, 1-5, 1-4, 1-3, or 1-2) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the Ig Fc region of a CD161 binding protein (e.g., an anti-CD161 antibody) comprises an IgG1 Fc region comprising one or more amino acid variation relative to a reference IgG1 Fc region.

In some embodiments, the IgG1 Fc region comprises an amino acid substitution at one or more (e.g., 1, 2, or 3) of amino acid positions S298, E333, K334, EU numbering according to Kabat. In some embodiments, the IgG1 Fc region comprises one or more (e.g., 1, 2, or 3) of the following amino acid substitutions S298A, E333A, and/or K334A, EU numbering according to Kabat.

In some embodiments, the IgG1 Fc region comprises an amino acid substitution at one or more (e.g., 1 or 2) of amino acid positions S239 and/or I332, EU numbering according to Kabat. In some embodiments, the IgG1 Fc region comprises one or more (e.g., 1 or 2) of the following amino acid substitutions S239D and/or I332E, EU numbering according to Kabat.

In some embodiments, the IgG1 Fc region comprises an amino acid substitution at one or more (e.g., 1 or 2) of amino acid positions P247 and/or A339, EU numbering according to Kabat. In some embodiments, the IgG1 Fc region comprises one or more (e.g., 1 or 2) of the following amino acid substitutions P247I and/or A339Q, EU numbering according to Kabat.

In some embodiments, the IgG1 Fc region comprises an amino acid substitution at one or more (e.g., 1, 2, or 3) of amino acid positions S239, A330, and/or I332, EU numbering according to Kabat. In some embodiments, the IgG1 Fc region comprises one or more (e.g., 1, 2, or 3) of the following amino acid substitutions S239D, A330L, and/or I332E, EU numbering according to Kabat.

In some embodiments, the IgG1 Fc region comprises an amino acid substitution at one or more (e.g., 1, 2, or 3) of amino acid positions G236, S239, and/or I332, EU numbering according to Kabat. In some embodiments, the IgG1 Fc region comprises one or more (e.g., 1, 2, or 3) of the following amino acid substitutions G236A, S239D, and/or I332E, EU numbering according to Kabat.

In some embodiments, the IgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the IgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the IgG1 Fc region comprises an amino acid substitution at one or more (e.g., 1, 2, 3, 4, or 5) of amino acid positions L235, F243, R292, Y300, and P396, EU numbering according to Kabat. In some embodiments, the IgG1 Fc region comprises one or more (e.g., 1, 2, 3, 4, or 5) of the following amino acid substitutions L235V, F243L, R292P, Y300L, and/or P396L, EU numbering according to Kabat.

In some embodiments, the IgG1 Fc region comprises an amino acid substitution at one or more (e.g., 1, 2, 3, 4, 5, 6, or 7) of amino acid positions L234, L235, G236, S239, H268, D270, and/or S298, EU numbering according to Kabat. In some embodiments, the IgG1 Fc region comprises one or more (e.g., 1, 2, 3, 4, 5, 6, or 7) of the following amino acid substitutions L234Y, L235Q, G236W, S239M, H268D, D270E, and/or S298A, EU numbering according to Kabat.

In some embodiments, the IgG1 Fc region comprises an amino acid substitution at one or more (e.g., 1, 2, 3, or 4) of amino acid positions D270, K326, A330, and/or K334, EU numbering according to Kabat. In some embodiments, the IgG1 Fc region comprises one or more (e.g., 1, 2, 3, or 4) of the following amino acid substitutions D270E, K326D, A330M, and/or K334E, EU numbering according to Kabat.

In some embodiments, the IgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the IgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the IgG1 Fc region comprises an amino acid substitution at one or more (e.g., 1, 2, or 3) of amino acid positions S239, 1332, and/or A330, EU numbering according to Kabat. In some embodiments, the IgG1 Fc region comprises one or more (e.g., 1, 2, or 3) of the following amino acid substitutions S239D, I332E, and/or A330L, EU numbering according to Kabat.

In some embodiments, the IgG1 Fc region comprises an amino acid substitution at one or more (e.g., 1, 2, 3, or 4) of amino acid positions S239, 1332, A330, and/or G236, EU numbering according to Kabat. In some embodiments, the IgG1 Fc region comprises one or more (e.g., 1, 2, 3, or 4) of the following amino acid substitutions S239D, I332E, A330L and/or G236A, EU numbering according to Kabat.

In some embodiments, the IgG1 Fc region comprises an amino acid substitution at one or more (e.g., 1, 2, or 3) of amino acid positions S239, 1332, and/or G326, EU numbering according to Kabat. In some embodiments, the IgG1 Fc region comprises one or more (e.g., 1, 2, or 3) of the following amino acid substitutions S239D, I332E, and/or G326A, EU numbering according to Kabat.

In some embodiments, the IgG1 Fc region comprises an amino acid substitution at amino acid position G326, EU numbering according to Kabat. In some embodiments, the IgG1 Fc region comprises a G326A amino acid substitution, EU numbering according to Kabat.

In some embodiments, the IgG1 Fc region comprises an amino acid substitution at one or more (e.g., 1, 2, or 3) of amino acid positions G236, S239, and/or I332, EU numbering according to Kabat. In some embodiments, the IgG1 Fc region comprises one or more (e.g., 1, 2, or 3) of the following amino acid substitutions G236A, S239D, and/or I332E, EU numbering according to Kabat.

In some embodiments, the IgG1 Fc region comprises an amino acid substitution at one or more (e.g., 1 or 2) of amino acid positions S239 and/or I332, EU numbering according to Kabat. In some embodiments, the IgG1 Fc region comprises one or more (e.g., 1 or 2) of the following amino acid substitutions S239D and/or I332E, EU numbering according to Kabat.

In some embodiments, the IgG1 Fc region comprises an amino acid substitution at one or more (e.g., 1 or 2) of amino acid positions K326 and/or E333, EU numbering according to Kabat. In some embodiments, the IgG1 Fc region comprises one or more (e.g., 1 or 2) of the following amino acid substitutions K326W and/or E333S, EU numbering according to Kabat.

In some embodiments, the IgG1 Fc region comprises an amino acid substitution at one or more (e.g., 1, 2, or 3) of amino acid positions S267, H268, and/or S324, EU numbering according to Kabat. In some embodiments, the IgG1 Fc region comprises one or more (e.g., 1, 2, or 3) of the following amino acid substitutions S267E, H268E, and/or S324T, EU numbering according to Kabat.

In some embodiments, the IgG1 Fc region comprises an amino acid substitution at one or more (e.g., 1, 2, or 3) of amino acid positions S298, E333, and/or K334, EU numbering according to Kabat. In some embodiments, the IgG1 Fc region comprises one or more (e.g., 1, 2, or 3) of the following amino acid substitutions S298A, E333A, and/or K334A, EU numbering according to Kabat.

In some embodiments, the IgG1 Fc region comprises an amino acid substitution at one or more (e.g., 1 or 2) of amino acid positions S239 and/or I332, EU numbering according to Kabat. In some embodiments, the IgG1 Fc region comprises one or more (e.g., 1 or 2) of the following amino acid substitutions S239D and/or I332E, EU numbering according to Kabat.

In some embodiments, the IgG1 Fc region comprises an amino acid substitution at one or more (e.g., 1 or 2) of amino acid positions P247 and/or A339, EU numbering according to Kabat. In some embodiments, the IgG1 Fc region comprises one or more (e.g., 1 or 2) of the following amino acid substitutions P247I and/or A339Q, EU numbering according to Kabat.

In some embodiments, the Ig Fc region of a CD161 binding protein (e.g., an anti-CD161 antibody) comprises one or more changes to the glycosylation.

In some embodiments, the Ig Fc region of a CD161 binding protein (e.g., an anti-CD161 antibody) is afucosylated. In some embodiments, the Ig Fc region of a CD161 binding protein (e.g., an anti-CD161 antibody) is afucosylated and a CD161 binding protein (e.g., an anti-CD161 antibody) comprising the afucosylated Ig Fc exhibits enhanced (e.g., increased) ADCC compared to a reference CD161 binding protein that is not afucosylated. Methods of producing afucosylated antibodies are known in the art. See, e.g., Pereira, Natasha A et al. “The “less-is-more” in therapeutic antibodies: Afucosylated anti-cancer antibodies with enhanced antibody-dependent cellular cytotoxicity.” mAbs vol. 10, 5 (2018): 693-711. doi:10.1080/19420862.2018.1466767, the entire contents of which is incorporated herein by reference for all purposes.

### 5.3.1.2 Reduced Ig Effector Function

In some embodiments, the Ig Fc region of a CD161 binding protein (e.g., described herein) exhibits a decrease in one or more Fc effector function relative to a reference (e.g., wild type) Ig Fc region. Exemplary Ig Fc effector functions include, but are not limited to, ADCC, ADCP, CDC, binding affinity to C1q, and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))).

In some embodiments, the Ig Fc region of a CD161 binding protein (e.g., described herein) is modified (e.g., comprises one or more variation (e.g., one or more amino acid substitution, deletion, addition, etc.); altered glycosylation)) (referred to herein as a “modified Ig Fc”). In some embodiments, the one or more variation (e.g., the one or more variation (e.g., the one or more amino acid substitution, deletion, addition, etc.; the altered glycosylation)) decreases or abolishes one or more Fc effector function, relative to a reference Ig Fc that does not comprise the modification (e.g., the one or more variation (e.g., the one or more amino acid substitution, deletion, addition, etc.; the altered glycosylation)).

In some embodiments, the modified Ig Fc fusion protein exhibits no detectable or decreased ADCC compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits no detectable or decreased CDC compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits no detectable or decreased ADCP compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγRIIa, and/or FcγRIIIa))) compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRI, FcγRIIa, and/or FcγRIIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRI compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to FcγRIIIa compared to a reference fusion protein that does not comprise the Ig Fc modification (e.g., the one or more variation (e.g., the one or more amino acid substitution, deletion, addition, etc.; the altered glycosylation)). In some embodiments, the modified Ig Fc fusion protein exhibits decreased or no binding affinity to C1q compared to a reference fusion protein that does not comprise the Ig Fc modification (e.g., the one or more variation (e.g., the one or more amino acid substitution, deletion, addition, etc.; the altered glycosylation)).

Amino acid substitutions that decrease or abolish one or more Ig Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

Table 5 below, provides exemplary amino acid substitutions (and combinations thereof) that can be utilized to increase one or more Fc effector function. Amino acids in Table 5 are numbered according to the EU numbering scheme. The effects on effector function set forth in Table 5 are exemplary only and not intended to be limiting. The amino acid substitutions set forth in Table 5 (except where noted) are with reference to an IgG1 Fc region. However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 5
Exemplary Ig Fc Variations and Glycoengineering
to Decrease Effector Function.

| | |
|---|---|
| | Exemplary Effects on |
| Change | Effector Function |

Amino Acid Substitutions

| | |
|---|---|
| L235E | Reduces ADCC |
| L234A/L235A | Reduces ADCC, ADCP, CDC |
| L234A/L235A/P329G | Reduces ADCC, ADCP |
| L234A/L235A/P329A | Reduces ADCC, ADCP |
| P331S/L234G/L235F | Reduces CDC |
| D265A | Reduces ADCC, ADCP |
| G237A | Reduces ADCP |
| E318A | Reduces ADCP |
| E233P | Reduces FcγRI, |
| | II and III binding |
| G236R/L328R | Reduces ADCC |
| D270A | Reduces CDC |
| K322A | Reduces CDC |
| P329A | Reduces CDC |
| P331A | Reduces CDC |
| V264A | Reduces CDC |
| F241A | Reduces CDC |
| N297A | Reduces CDC |
| N297G | Reduces CDC |
| N297Q | Reduces CDC |
| IgG4 - S228P/L235E | Reduces FcγRI binding |
| IgG4 - S228P/F234A/L235A | Reduces ADCC, CDC |
| IgG4 - S228P/F234A/L235G | Reduces ADCC, CDC |
| IgG4 - S228P/F234A/L235E | Reduces ADCC, CDC |
| IgG2 - H268Q/V309L/A330S/P331S | Reduces ADCC, ADCP, CDC |

Glycoengineering

| | |
|---|---|
| High Mannose | Reduces CDC |

In some embodiments, the Ig Fc (e.g., IgG1 Fc) region of a CD161 binding protein (e.g., described herein) comprises any one or more of the amino acid substitutions set forth in Table 5 (set forth in any set). In some embodiments, the Ig Fc (e.g., IgG1 Fc) region of a CD161 binding protein (e.g., described herein) comprises any one or more of the sets of amino acid substitutions set forth in Table 5. In some embodiments, the Ig Fc (e.g., IgG1 Fc) region of a CD161 binding protein (e.g., described herein) comprises any one or more of the glycosylation changes set forth in Table 5.

In some embodiments, the Ig Fc (e.g., IgG1 Fc) region of a CD161 binding protein (e.g., described herein) comprises an amino acid substitution at any one or more (e.g., 1, 2, 3, 4, 5, 6, 7, 8, 9, or 10, or more) of amino acid positions L234, L235, P329, P331, D265, G237, E318, E233, G236, L328, D270, K322, V264, F241, and/or N297. In some embodiments, the Ig Fc (e.g., IgG1 Fc) region of a CD161 binding protein (e.g., described herein) comprises an amino acid substitution at from about 1-10 (e.g., 1-9, 1-8, 1-7, 1-6, 1-5, 1-4, 1-3, or 1-2) of the following amino acid positions L234, L235, P329, P331, D265, G237, E318, E233, G236, L328, D270, K322, V264, F241, and/or N297.

In some embodiments, the Ig Fc (e.g., IgG1 Fc) region of a CD161 binding protein (e.g., described herein) comprises one or more (e.g., 1, 2, 3, 4, 5, 6, 7, 8, 9, or 10, or more) of the following amino acid substitutions L234A, L234G, L235F, L235E, L235A, P329G, P329A, P331S, D265A, G237A, E318A, E233P, G236R, L328R, D270A, K322A, V264A, F241A, N297A, N297G, and/or N297Q. In some embodiments, the Ig Fc (e.g., IgG1 Fc) region of a CD161 binding protein (e.g., described herein) comprises from about 1-10 (e.g., 1-9, 1-8, 1-7, 1-6, 1-5, 1-4, 1-3, or 1-2) of the following amino acid substitutions L234A, L234G, L235F, L235E, L235A, P329G, P329A, P331S, D265A, G237A, E318A, E233P, G236R, L328R, D270A, K322A, V264A, F241A, N297A, N297G, or N297Q.

In some embodiments, the Ig Fc region of a CD161 binding protein (e.g., described herein) comprises an IgG1 Fc region comprising one or more amino acid variation.

In some embodiments, the IgG1 Fc region comprises an amino acid substitution at amino acid position L234, EU numbering according to Kabat. In some embodiments, the IgG1 Fc region comprises a L234A or L234G amino acid substitution, EU numbering according to Kabat.

In some embodiments, the IgG1 Fc region comprises an amino acid substitution at amino acid position L235, EU numbering according to Kabat. In some embodiments, the IgG1 Fc region comprises a L235A, L235G, L235E, or L235F amino acid substitution, EU numbering according to Kabat.

In some embodiments, the IgG1 Fc region comprises an amino acid substitution at amino acid position P329, EU numbering according to Kabat. In some embodiments, the IgG1 Fc region comprises a P329A or P329G amino acid substitution, EU numbering according to Kabat.

In some embodiments, the IgG1 Fc region comprises an amino acid substitution at one or more (e.g., 1 or 2) of amino acid positions L234 and/or L235, EU numbering according to Kabat. In some embodiments, the IgG1 Fc region comprises one or more (e.g., 1, 2, or 3) of the following amino acid substitutions L234A and/or L235A, EU numbering according to Kabat.

In some embodiments, the IgG1 Fc region comprises an amino acid substitution at one or more (e.g., 1, 2, or 3) of amino acid positions L234, L235, and/or P329, EU numbering according to Kabat. In some embodiments, the IgG1 Fc region comprises one or more (e.g., 1, 2, or 3) of the following amino acid substitutions L234A, L235A, and/or P329G, EU numbering according to Kabat. In some embodiments, the IgG1 Fc region comprises one or more (e.g., 1, 2, or 3) of the following amino acid substitutions L234A, L235A, and/or P329A, EU numbering according to Kabat.

In some embodiments, the IgG1 Fc region comprises an amino acid substitution at one or more (e.g., 1, 2, or 3) of amino acid positions P331, L234, and/or L235, EU numbering according to Kabat. In some embodiments, the IgG1 Fc region comprises one or more (e.g., 1, 2, or 3) of the following amino acid substitutions P331S, L234G, and/or L235F, EU numbering according to Kabat.

In some embodiments, the IgG1 Fc region comprises an amino acid substitution at amino acid position D265, EU numbering according to Kabat. In some embodiments, the IgG1 Fc region comprises a D235A amino acid substitution, EU numbering according to Kabat.

In some embodiments, the IgG1 Fc region comprises an amino acid substitution at amino acid position G237, EU numbering according to Kabat. In some embodiments, the IgG1 Fc region comprises a G237A amino acid substitution, EU numbering according to Kabat.

In some embodiments, the IgG1 Fc region comprises an amino acid substitution at amino acid position E318, EU numbering according to Kabat. In some embodiments, the IgG1 Fc region comprises a E318A amino acid substitution, EU numbering according to Kabat.

In some embodiments, the IgG1 Fc region comprises an amino acid substitution at amino acid position E233, EU numbering according to Kabat. In some embodiments, the IgG1 Fc region comprises a E233P amino acid substitution, EU numbering according to Kabat.

In some embodiments, the IgG1 Fc region comprises an amino acid substitution at amino acid position D270, EU numbering according to Kabat. In some embodiments, the IgG1 Fc region comprises a D270A amino acid substitution, EU numbering according to Kabat.

In some embodiments, the IgG1 Fc region comprises an amino acid substitution at amino acid position K322, EU numbering according to Kabat. In some embodiments, the IgG1 Fc region comprises a K322A amino acid substitution, EU numbering according to Kabat.

In some embodiments, the IgG1 Fc region comprises an amino acid substitution at amino acid position P331, EU numbering according to Kabat. In some embodiments, the IgG1 Fc region comprises a P331A amino acid substitution, EU numbering according to Kabat.

In some embodiments, the IgG1 Fc region comprises an amino acid substitution at amino acid position F241, EU numbering according to Kabat. In some embodiments, the IgG1 Fc region comprises a F241A amino acid substitution, EU numbering according to Kabat.

In some embodiments, the IgG1 Fc region comprises an amino acid substitution at amino acid position N297, EU numbering according to Kabat. In some embodiments, the IgG1 Fc region comprises a N297A, N297G, or N297Q amino acid substitution, EU numbering according to Kabat.

In some embodiments, the IgG1 Fc region comprises an amino acid substitution at one or more (e.g., 1 or 2) of amino acid positions G236 and/or L328, EU numbering according to Kabat. In some embodiments, the IgG1 Fc region comprises one or more (e.g., 1, 2, or 3) of the following amino acid substitutions G236R and/or L328R, EU numbering according to Kabat.

In some embodiments, the IgG4 Fc region comprises an amino acid substitution at amino acid position S228, EU numbering according to Kabat. In some embodiments, the IgG1 Fc region comprises an S228P amino acid substitution, EU numbering according to Kabat.

In some embodiments, the IgG4 Fc region comprises an amino acid substitution at amino acid position F234, EU numbering according to Kabat. In some embodiments, the IgG1 Fc region comprises an F234A amino acid substitution, EU numbering according to Kabat.

In some embodiments, the IgG4 Fc region comprises an amino acid substitution at amino acid position L235A, EU numbering according to Kabat. In some embodiments, the IgG1 Fc region comprises an L235A, L235G, or L235E amino acid substitution, EU numbering according to Kabat.

In some embodiments, the IgG4 Fc region comprises an amino acid substitution at one or more (e.g., 1 or 2) of amino acid positions S228 and/or L235, EU numbering according to Kabat. In some embodiments, the IgG4 Fc region comprises one or more (e.g., 1, 2, or 3) of the following amino acid substitutions S228P and/or L235E, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235A, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235G, EU numbering according to Kabat.

In some embodiments, the IgG4 Fc region comprises an amino acid substitution at one or more (e.g., 1, 2, or 3) of amino acid positions S228, F234, and/or L235, EU numbering according to Kabat. In some embodiments, the IgG4 Fc region comprises one or more (e.g., 1, 2, or 3) of the following amino acid substitutions S228P, F234A, and/or L235E, EU numbering according to Kabat.

In some embodiments, the IgG2 Fc region comprises an amino acid substitution at one or more (e.g., 1, 2, 3, or 4) of amino acid positions H268, V309, A330, and/or P331, EU numbering according to Kabat. In some embodiments, the IgG2 Fc region comprises one or more (e.g., 1, 2, 3, or 4) of the following amino acid substitutions H268Q, V309L, A330S, and/or P331S, EU numbering according to Kabat.

In some embodiments, the Ig Fc region of a CD161 binding protein (e.g., described herein) comprises one or more changes to the glycosylation. In some embodiments, the Ig Fc region of a CD161 binding protein (e.g., described herein) has increased high mannose glycosylation.

### 5.3.2 Promotion of Heterodimerization

As described herein, in some embodiments, the antibody comprises a first and second Fc region. In some embodiments, the first Ig Fc region and the second Ig Fc region each comprise one or more amino acid modifications relative to each other to promote heterodimerization. IgG derived heterodimeric formats can be generated by methods known in the art, e.g., by forced heavy chain heterodimerization. Forced heavy chain heterodimerization can be obtained using known methods in the art, e.g., knob-in-hole or strand exchange engineered domains (SEED), see, e.g., Ji-Hee et al., “Immunoglobulin Fc Heterodimer Platform Technology: From Design to Applications in Therapeutic Antibodies and Proteins” Frontiers in Immunology, v7 (article 394) (2016) DOI=10.3389/fimmu.2016.00394 (hereinafter “Ji-Hee 2016”), the entire contents of which is incorporated by reference herein for all purposes.

In some embodiments, an interface of the first and the second Ig Fc regions is varied, e.g., introduction of an amino acid substitution, to increase heterodimerization, e.g., relative to a non-modified interface, e.g., a naturally occurring interface. For example, dimerization of the first and second Ig Fc regions can be enhanced by providing an Ig Fc interface of a first and a second Fc region with one or more of: a paired protuberance-cavity (“knob-in-hole”), an electrostatic interaction, or a strand-exchange, such that a greater ratio of heteromultimer to homomultimer forms, e.g., relative to a non-modified interface.

Knob-in-Hole amino acid pairing modifications are known in the art, and described in e.g., U.S. Pat. Nos. 5,731,116; 7,476,724; Ji-Hee 2016; and Ridgway, J. “‘Knobs-into-holes’ engineering of antibody CH3 domains for heavy chain heterodimerization” et al. Prot. Engineering 9(7):617-621 (1996), the full contents of each of which is incorporated by reference herein. Generally, Knob-in-Hole comprises 1) introducing one or more amino acid substitutions in the CH3 domain of one or both of the first and second subject Ig Fc regions to promote heterodimerization; and 2) combining the modified Ig Fc regions under conditions that promote heterodimerization. “Knobs” are typically created by substituting a small amino acid in a parental Ig Fc region with a larger amino acid (e.g., T366Y or T366W); “holes” are created by substituting a larger residue in a parental Ig Fc region with a smaller amino acid (e.g., Y407T, T366S, 11368A, or Y407V). Exemplary Knob-in-Hole mutations include S354C, T366W in the “knob” Ig Fc region and Y349C, T366S, L368A, Y407V in the “hole” Ig Fc region. Other exemplary Knob-in-Hole mutations, which can be incorporated into any one or more of the embodiments, are provided in Table 6, with additional exemplary optional stabilizing Ig Fc cysteine mutations.

TABLE 6
Exemplary Knob-in-hole and Stabilizing Cysteine Modifications.

| | | |
|---|---|---|
| Amino Acid Position | Knob Ig Fc | Hole Ig Fc |
| (EU numbering according | Amino Acid | Amino Acid |
| to Kabat) | Substitution | Substitution |

Knob-in Hole Amino Acid Substitutions

| | | |
|---|---|---|
| T366 | T366W | T366S |
| L368 | — | L368A |
| Y407 | — | Y407V |

Stabilizing Cysteine Amino Acid Substitutions

| | | |
|---|---|---|
| S354 | S354C | — |
| Y349 | — | Y349C |

As described herein, in some embodiments, the antibody comprises a first Fc region and a second Ig Fc region.

In some embodiments, the amino acid sequence of the first Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 3). In some embodiments, the amino acid sequence of the first hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 3).

In some embodiments, the amino acid sequence of the first Fc region comprises each of the following amino acid substitutions: T366W and a S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 3).

In some embodiments, the amino acid sequence of the second Fc region comprises a T366W amino acid substitution, EU numbering according to Kabat; and the second the amino acid sequence of the Fc region comprises each of the following amino acid substitutions: T366S, L368A, and Y407V, EU numbering according to Kabat; each relative to the amino acid sequence of an exemplary reference Ig Fc region (e.g., a reference Ig Fc region set forth in Table 3). In some embodiments, the amino acid sequence of the second hIg further comprises a S354C amino acid substitution, EU numbering according to Kabat; and the amino acid sequence of the second Fc region comprises a Y349C amino acid substitution, EU numbering according to Kabat; each relative to the amino acid sequence of an exemplary reference Ig Fc region (e.g., a reference Ig Fc region set forth in Table 3).

In some embodiments, the amino acid sequence of the second Fc region comprises each of the following amino acid substitutions: T366W and S354C, EU numbering according to Kabat; and the second the amino acid sequence of the Fc region comprises each of the following amino acid substitutions: T366S, L368A, Y407V, and Y349C, EU numbering according to Kabat; each relative to the amino acid sequence of an exemplary reference Ig Fc region (e.g., a reference Ig Fc region set forth in Table 3).

In some embodiments, the amino acid sequence of the first Ig Fc region comprises a W amino acid at position T366, EU numbering according to Kabat; and the amino acid sequence of the second Ig Fc region comprises a S amino acid at position T366, an A amino acid at position L368, and a V amino acid at position Y407, EU numbering according to Kabat.

In some embodiments, the amino acid sequence of the first Ig Fc region comprises a W amino acid at position T366 and a C amino acid at position S354, EU numbering according to Kabat; and the amino acid sequence of the second Ig Fc region comprises a S amino acid at position T366, an A amino acid at position L368, a V amino acid at position Y407, and a C amino acid at position Y349, EU numbering according to Kabat.

### 5.4 Conjugates and Fusion Proteins

In some embodiments, a CD161 binding protein described herein (e.g., an anti-CD161 antibody) is conjugated or fused to one or more heterologous moiety. As such, further provided herein are fusion proteins comprising a CD161 binding protein described herein (e.g., an anti-CD161 antibody) fused (directly or indirectly (via a linker)) to one or more heterologous protein. Further provided herein are conjugates comprising a CD161 binding protein described herein (e.g., an anti-CD161 antibody) conjugated (directly or indirectly (via a linker)) to one or more heterologous moiety.

In some embodiments, the heterologous moiety is a protein, peptide, polynucleotide (e.g., DNA, RNA, DNA/RNA hybrid), small molecule, carbohydrate, lipid, or synthetic polymer.

In some embodiments, the heterologous protein comprises an IgG (e.g., IgG1, IgG2 (e.g., IgG2a or IgG2b), IgG3, IgG4), IgE, IgM, IgD, or IgA (e.g., IgAQ1 or IgA2) antibody. In some embodiments, the heterologous polypeptide is an antibody (e.g., a full-length antibody, scFv, Fab, F(ab′)2, Fab′, Fv, single domain antibody (e.g., a VHH), scFv-Fc, Fab-Fc, and/or single domain antibody-Fc (e.g., VHH-Fc)). In some embodiments, the heterologous protein comprises one or more of a monoclonal antibody, monospecific antibody, multispecific antibody, human antibody, humanized antibody, chimeric antibody, and/or murine antibody, or a functional fragment or functional variant of any of the foregoing. In some embodiments, the antibody is a chimeric antigen receptor (e.g., described herein). In some embodiments, the antibody is a T-cell engager (e.g., described herein).

In some embodiments, the heterologous moiety is a therapeutic agent. In some embodiments, the heterologous moiety is a chemotherapeutic agent, a cytotoxic agent, an anti-cancer agent, or a radioactive isotope, or any combination thereof. In some embodiments, the heterologous moiety is a targeting moiety. In some embodiments, the heterologous moiety is a detectable moiety. In some embodiments, the heterologous moiety is a diagnostic moiety. In some embodiments, the heterologous moiety is a fluorescent moiety. Fluorescent or chemiluminescent labels include fluorophores such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

In some embodiments, one or more polypeptide of a CD161 binding protein (or one or more polypeptide thereof) or polypeptide (e.g., described herein) comprises a signal peptide operably connected to the N-terminus. Commonly utilized signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art.

### 5.4.1 Chimeric Antigen Receptors

In some embodiments, a CD161 binding protein described herein is part of a chimeric antigen receptor (CAR). In some embodiments, a CD161 binding protein described herein is the extracellular antigen-binding domain of a CAR. Standard CAR domains are known in art, including, e.g., transmembrane domains and intracellular signaling domains. See, e.g., WO2024056809, WO2023240064A1, and WO2023205148A1, WO2023133092A1, the entire contents of each of which is incorporated herein by reference for all purposes.

Exemplary transmembrane domains include, e.g., the alpha, beta or zeta chain of T-cell receptor, CD28, CD3 epsilon, CD45, CD4, CD5, CD8 (for example, CD8 alpha, CD8 beta), CD9, CD 16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CDlla/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDlld, ITGAE, CD103, ITGAL, CDlla, LFA-1, ITGAM, CDllb, ITGAX, CDllc, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD 150, IPO-3), BLAME (SLAMF8), SELPLG (CD 162), LTBR, LAT, GADS, SLP-76, PAG/Cbp, CD 19a, and a ligand that specifically binds with CD83. In some instances, the transmembrane domain can be attached to the extracellular region of the CAR, for example, the antigen-binding domain of the CAR, via a hinge, for example, a hinge from a human protein. For example, in some embodiments, the hinge can be a human Ig (immunoglobulin) hinge, for example, an IgG4 hinge, or a CD8a hinge.

Exemplary intracellular signaling domains include, e.g., the cytoplasmic sequences of the T cell receptor (TCR) and co-receptors that act in concert to initiate signal transduction following antigen receptor engagement, as well as any derivative or variant of these sequences and any recombinant sequence that has the same functional capability. In some embodiments, the intracellular signaling domain comprises a primary signaling domain and one or more costimulatory signaling domain. Exemplary primary signaling domains, include, e.g., intracellular signaling domains of TCR zeta, FcR gamma, FcR beta, CD3 gamma, CD3 delta, CD3 epsilon, CD5, CD22, CD79a, CD79b, CD278 (also known as “ICOS”), FccRI, DAP10, DAP12, CD32, and CD66d. Exemplary of proteins with costimulatory domains suitable for use in CAR described herein include, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, 0X40, CD2, CD7, CD27, CD28, CD30, CD40, CDS, ICAM-1, LFA-1 (CD1 la/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRFl), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD lid, ITGAE, CD 103, ITGAL, CDlla, LFA-1, ITGAM, CDllb, ITGAX, CDIIc, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, CD 19a, and a ligand that specifically binds with CD83, and the like.

### 5.4.2 T-Cell Engagers

In some embodiments, a CD161 binding protein described herein is part of a T cell engager (i.e., an agent (e.g., antibody) that binds at one molecule (e.g., protein) expressed on the surface of a T-cell). In some embodiments, the T-cell engager is multispecific (e.g., bispecific, trispecific, etc). In some embodiments, the T-cell engager is bispecific. In some embodiments, the T-cell engager is trispecific. In some embodiments, the T-cell engager binds to least one molecule (e.g., protein) expressed on the surface of a T cell and at least one molecule expressed on the surface of a non-T-cell. In some embodiments, the CD161 binding protein acts as the T-cell engager.

### 5.4.3 Half-Life Extension Moieties

In some embodiments, the heterologous moiety (e.g., protein) is a half-life extension moiety (e.g., protein). Various half-life extension moieties are known in the art. See, e.g., Ko S, Jo M, Jung S T. Recent Achievements and Challenges in Prolonging the Serum Half-Lives of Therapeutic IgG Antibodies Through Fc Engineering. BioDrugs. 2021; 35(2):147-157. doi:10.1007/s40259-021-00471-0 (hereinafter “Ko 2021”); Bech, E. M., Pedersen, S. L., & Jensen, K. J. (2018). Chemical Strategies for Half-Life Extension of Biopharmaceuticals: Lipidation and Its Alternatives. ACS medicinal chemistry letters, 9(7), 577-580. https://doi.org/10.1021/acsmedchemlett.8b00226 (hereinafter “Bech 2018”); Mester S, Evers M, Meyer S, et al. Extended plasma half-life of albumin-binding domain fused human IgA upon pH-dependent albumin engagement of human FcRn in vitro and in vivo. MAbs. 2021; 13(1):1893888. doi:10.1080/19420862.2021.1893888 (hereinafter “Mester 2021”); Kontermann R E. Strategies for extended serum half-life of protein therapeutics. Curr Opin Biotechnol. 2011; 22(6):868-876. doi:10.1016/j.copbio.2011.06.012 (hereinafter “Kontermann 2011”); Strohl W. R. (2015). Fusion Proteins for Half-Life Extension of Biologics as a Strategy to Make Biobetters. BioDrugs: clinical immunotherapeutics, biopharmaceuticals and gene therapy, 29(4), 215-239. https://doi.org/10.1007/s40259-015-0133-6; Zaman R, Islam R A, Ibnat N, et al. Current strategies in extending half-lives of therapeutic proteins. J Control Release. 2019; 301:176-189. doi:10.1016/j.jconrel.2019.02.016; Chen C, Constantinou A, Chester K A, et al. Glycoengineering approach to half-life extension of recombinant biotherapeutics. Bioconjug Chem. 2012; 23(8):1524-1533. doi:10.1021/bc200624a; Gupta, Vijayalaxmi et al. “Protein PEGylation for cancer therapy: bench to bedside.” Journal of cell communication and signaling vol. 13, 3 (2019): 319-330. doi:10.1007/s12079-018-0492-0; Martin Schlapschy, et al, PASylation: a biological alternative to PEGylation for extending the plasma half-life of pharmaceutically active proteins, Protein Engineering, Design and Selection, Volume 26, Issue 8, August 2013, Pages 489-501, https://doi.org/10.1093/protein/gzt023; Strohl, William R. “Fusion Proteins for Half-Life Extension of Biologics as a Strategy to Make Biobetters.” BioDrugs: clinical immunotherapeutics, biopharmaceuticals and gene therapy vol. 29, 4 (2015): 215-39. doi:10.1007/s40259-015-0133-6; the entire contents of each of which are incorporated by reference herein for all purposes.

Exemplary half-life extension moieties include, but are not limited to, an immunoglobulin (e.g., human Ig (hIg), murine Ig (mIg)), a fragment of an Ig (e.g., hIg, mIg), an Ig (e.g., hIg, mIg) constant region, a fragment of an Ig (e.g., hIg, mIg) constant region, an Ig (e.g., hIg, mIg) Fc region, human transferrin, a human transferrin binding moiety (e.g., small molecule, lipid, protein, peptide, etc.), human serum albumin (HSA), a fragment of HSA, an HSA binding moiety (e.g., small molecule, lipid, protein, peptide, etc.) (e.g., an antibody, a Streptococcal protein G (see, e.g., Mester 2021), polyethylene glycol (PEG) (and polymers thereof) (e.g., pegylation), lipids, small molecules, carbohydrates (e.g., glycosylation, polysialic acid (polysialylation), hydroxyethyl starch (HES) (HESylation), heparosan (HEPylation)).

In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, a mIg, a fragment of a mIg, one or more mIg heavy chain constant region, a fragment of a mIg constant region, a mIg Fc region, human transferrin, a fragment of human transferrin, a human transferrin binding protein (e.g., an antibody) HSA, and HSA binding proteins (e.g., an antibody, a Streptococcal protein G). In some embodiments, the half-life extension polypeptide comprises an Ig Fc region (e.g., hIg Fc region). In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)). See, e.g., § 5.3.

In some embodiments, half-life extension is mediated through one or more of lipidation, glycosylation, polysialylation, HESylation, HEPylation, and/or pegylation. In some embodiments, half-life extension is mediated through one or more of lipidation, HESylation, HEPylation, and/or pegylation. In some embodiments, half-life extension is mediated through glycosylation. In some embodiments, half-life extension is mediated through polysialylation.

In some embodiments, the half-life extension moiety comprises one or more lipids. See, e.g., Bech 2018. In some embodiments, the half-life extension moiety comprises one or more post translational modifications (e.g., glycosylation, polysialylation, etc.).

In some embodiments, the half-life extension moiety (e.g., protein) is altered (e.g., compared to a reference half-life extension moiety (e.g., protein)) to further enhance half-life of the fusion protein or conjugate. Various alterations to known half-life extension moieties (e.g., proteins) are known in the art. See, e.g., Ko 2021, Bech 2018, Mester 2021, and Kontermann 2011. Modifications include, e.g., amino acid variations (e.g., substitutions, additions, deletions) and post translational modifications (e.g., altered lipidation, glycosylation, polysialylation, HESylation, HEPylation, pegylation, etc.).

The immunoreceptor inhibitory protein described herein fused or conjugated to a half-life extending moiety or a half-life extending moiety can be evaluated for their pharmacokinetic properties utilizing standard in vivo methods known in the art. See, e.g., Avery, Lindsay B et al. “Utility of a human FcRn transgenic mouse model in drug discovery for early assessment and prediction of human pharmacokinetics of monoclonal antibodies.” mAbs vol. 8, 6 (2016):1064-78. doi:10.1080/19420862.2016.1193660; Conner, Christopher M et al. “A precisely humanized FCRN transgenic mouse for preclinical pharmacokinetics studies.” Biochemical pharmacology vol. 210 (2023):115470. doi:10.1016/j.bcp.2023.115470; and Kathryn Ball et al., PK and Biodistribution of Therapeutic Proteins, Drug Metabolism and Disposition Jun. 1, 2022, 50 (6) 858-866; DOI: https://doi.org/10.1124/dmd.121.000463 (hereinafter “Ball 2022”), the entire contents of each of which are incorporated herein by reference for all purposes.

### 5.4.4 Linkers

As described herein, the heterologous moiety of conjugates and fusion proteins and polypeptides described herein can be directly operably connected or indirectly operably connected to the CD161 binding protein (e.g., described herein).

In some embodiments, the heterologous moiety of conjugates and fusion proteins and polypeptides described herein is directly operably connected to the CD161 binding protein (e.g., described herein). In some embodiments, the heterologous moiety of conjugates and fusion proteins and polypeptides described herein is indirectly operably connected to the CD161 binding protein (e.g., described herein). In some embodiments, the heterologous moiety of conjugates and fusion proteins and polypeptides described herein is indirectly operably connected to the CD161 binding protein (e.g., described herein) through a linker.

Linkers can be peptide linkers or non-peptide linkers. In some embodiments, the linker is derived from a crosslinking reagent. In some embodiments, the linker is cleavable. In some embodiments, the linker is non-cleavable. In some embodiments, the linker is synthetic.

### 5.4.4.1 Non-Peptide Linkers

In some embodiments, the linker is a non-peptide linker. In some embodiments, the linker is a chemical linker. In some embodiments, the linker is synthetic. In some embodiments, the linker is derived from a crosslinking reagent. In some embodiments, the linker is cleavable. In some embodiments, the linker is non-cleavable.

A variety of non-peptide linkers are known in the art that can be utilized to conjugate a heterologous moiety to a CD161 binding protein described herein. Suitable linkers have two reactive termini, one for conjugation to a CD161 binding protein described herein and one for conjugation to the heterologous moiety.

The terminus of the linker that is conjugated to a protein CD161 binding protein described herein can be a site that is capable of conjugation to the protein through a cysteine thiol or lysine amine group on the protein, and as such comprises a thiol reactive group (e.g., as in maleimide, acrylamide, vinyl sulfone, bromide and tosylate linkers) or an amine-reactive group. The cysteine thiol or lysine amine group can be naturally occurring or engineered (e.g., proteins comprising engineered cysteine or lysine amino acid residues).

In some embodiments, the linker comprises one or more of polyethylene glycol (PEG), maleimide, acrylamide, vinyl sulfone, bromide or tosylate. In some embodiments, the linker is derived from a crosslinking reagent such as N-succinimidyl-3-(2-pyridyldithio) propionate (SPDP), N-succinimidyl 4-(2-pyridyldithio) pentanoate (SPP), N-succinimidyl 4-(2-pyridyldithio) butanoate (SPDB), N-succinimidyl-4-(2-pyridyldithio)-2-sulfo-butanoate (sulfo-SPDB), N-succinimidyl iodoacetate (SIA), N-succinimidyl (4-iodoacetyl)aminobenzoate (SIAB), maleimide PEG NHS, N-succinimidyl 4-(maleimidomethyl)cyclohexanecarboxylate (SMCC), N-sulfosuccinimidyl 4-(maleimidomethyl)cyclohexanecarboxylate (sulfo-SMCC) or 2,5-dioxopyrrolidin-1-yl 17-(2,5-dioxo-2,5-dihydro-1H-pyrrol-1-yl)-5,8,11,14-tetraoxo-4,7,10,13-tetraazaheptadecan-1-oate (CX1-1).

Exemplary linkers are for example described in e.g., Zheng Su et al., Antibody-drug conjugates: Recent advances in linker chemistry, Acta Pharmaceutica Sinica B, Volume 11, Issue 12, 3889-3907 (2021); and Sheyi, R.; de la Torre, B. G.; Albericio, F. Linkers: An Assurance for Controlled Delivery of Antibody-Drug Conjugate. Pharmaceutics 2022, 14, 396. https://doi.org/10.3390/pharmaceutics14020396, the entire contents of each of which is incorporated herein by reference for all purposes.

### 5.4.4.2 Peptide Linkers

In some embodiments, the heterologous moiety (e.g., heterologous protein) is directly operably connected to the CD161 binding protein (e.g., described herein) via a peptide bond. In some embodiments, the heterologous moiety is indirectly operably connected to the CD161 binding protein (e.g., described herein) via a peptide linker.

In some embodiments, the linker is a peptide linker. In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

The amino acid sequence of exemplary peptide linkers, which can be incorporated in one or more of the embodiments described herein (e.g., fusion proteins and polypeptides, conjugates), is set provided in Table 7.

TABLE 7
The Amino Acid Sequence of Exemplary Peptide
Linkers

| | | SEQ |
|---|---|---|
| | | ID |
| Description | Amino Acid Sequence | NO |
| Linker A | GGGS | 39 |
| Linker B | GGGSGGGS | 40 |
| Linker C | GGGSGGGSGGGS | 41 |
| Linker D | GGGSGGGSGGGSGGGS | 42 |
| Linker E | GGGGS | 43 |
| Linker F | GGGGSGGGGS | 44 |
| Linker G | GGGGSGGGGSGGGGS | 45 |
| Linker H | GGGGSGGGGSGGGGSGGGGS | 46 |
| Linker I | GGGGGGGS | 47 |
| Linker J | GGGGGGGSGGGGGGGS | 48 |
| Linker K | GGGGGGGSGGGGGGGSGGGGGGGS | 49 |
| Linker L | GGGGGGGSGGGGGGGSGGGGGGGSGGGGGGGS | 50 |
| Linker M | SGGGG | 51 |
| Linker N | SGGGGSGGGG | 52 |
| Linker O | SGGGGSGGGGSGGGG | 53 |
| Linker P | SGGGGSGGGGSGGGGSGGGG | 54 |
| Linker Q | GGGGGGS | 55 |
| Linker R | GGGGGGSGGGGGGS | 56 |
| Linker S | GGGGGGSGGGGGGSGGGGGGS | 57 |
| Linker T | GGGGGGSGGGGGGSGGGGGGSGGGGGGS | 58 |

In some embodiments, the amino acid sequence of the peptide linker comprises or consists of the amino acid sequence of any one of the linkers set forth in Table 7. In some embodiments, the amino acid sequence of the peptide linker comprises or consists of the amino acid sequence of any one of the linkers set forth in Table 7, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 7, comprising 1, 2, or 3 amino acid variations (e.g., substitutions, deletions, additions). In some embodiments, the amino acid sequence of the peptide linker comprises or consists of the amino acid sequence of any one of the linkers set forth in Table 7, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 7, comprising 1, 2, or 3 amino acid substitutions.

In some embodiments, the amino acid sequence of the peptide linker comprises or consists of the amino acid sequence of any one of SEQ ID NOS: 39-58. In some embodiments, the amino acid sequence of the peptide linker comprises or consists of the amino acid sequence of any one of SEQ ID NOS: 39-58, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 39-58, comprising 1, 2, or 3 amino acid variations (e.g., substitutions, deletions, additions). In some embodiments, the amino acid sequence of the peptide linker comprises or consists of the amino acid sequence of any one of SEQ ID NOS: 39-58, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 39-58, comprising 1, 2, or 3 amino acid substitutions.

### 5.4.5 Orientation

The heterologous moiety (e.g., heterologous polypeptide) and the CD161 binding protein (e.g., described herein) of a conjugate or fusion (e.g., described herein) can be arranged in any configuration or order as long as the CD161 binding protein (e.g., described herein) maintains the ability to mediate its function and the heterologous moiety (e.g., heterologous polypeptide) can mediate its function.

The heterologous moiety can be attached (e.g., conjugated or fused) to the N-terminus, C-terminus, or at an internal site (i.e., between the N- and C-terminus) of the CD161 binding protein (e.g., one or more polypeptide of a CD161 binding protein).

In some embodiments, the heterologous moiety is attached (e.g., conjugated or fused) to the N-terminus of the CD161 binding protein (e.g., one or more polypeptide of the CD161 binding protein) or polypeptide. In some embodiments, the heterologous moiety is attached (e.g., conjugated or fused) to the C-terminus of the CD161 binding protein (e.g., one or more polypeptide of the CD161 binding protein). In some embodiments, the heterologous moiety is attached (e.g., conjugated or fused) at an internal site (i.e., between the N- and C-terminus) of the CD161 binding protein (e.g., one or more polypeptide of the CD161 binding protein).

### 5.5 Multimeric CD161 Binding Proteins

In some embodiments, the CD161 binding proteins (e.g., described herein) (or conjugates or fusions comprising the same) are multimeric (e.g., dimeric, trimeric, tetrameric, etc.) proteins comprising at least two, three, or four polypeptides.

In some embodiments, the CD161 binding protein comprises at least two, three, or four polypeptides. In some embodiments, the CD161 binding protein is dimeric (i.e., comprises two polypeptides). In some embodiments, the CD161 binding protein is trimeric (i.e., comprises three polypeptides). In some embodiments, the CD161 binding protein is tetrameric (i.e., comprises four polypeptides).

In some embodiments, two of the polypeptides associate via covalent or non-covalent interactions. In some embodiments, two of the polypeptides associate via at least one covalent interaction. In some embodiments, two of the polypeptides associate via one or more disulfide bond. In some embodiments, two of the polypeptides associate via 1, 2, 3, 4, or more disulfide bonds.

In some embodiments, the CD161 binding protein comprises a full-length antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the amino acid sequence of the first heavy chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the first light chain is at least 85%, 86%, 87%, 88%, 89%, 90%, 91%, 92%, 93%, 94%, 95%, 96%, 97%, 98%, 99%, or 100% identical to the amino acid sequence of the second heavy chain. In some embodiments, the amino acid sequence of the VH region of the first heavy chain is 100% identical to the amino acid sequence of the VH region of the second heavy chain; and the amino acid sequence of the first heavy chain outside of the VH region is at least 85%, 86%, 87%, 88%, 89%, 90%, 91%, 92%, 93%, 94%, 95%, 96%, 97%, 98%, 99%, or 100% identical to the amino acid sequence of the second heavy chain outside of the VH region of the second heavy chain. In some embodiments, the amino acid sequence of the VL region of the first light chain is 100% identical to the amino acid sequence of the VL region of the second light chain; and the amino acid sequence of the first light chain outside of the VL region is at least 85%, 86%, 87%, 88%, 89%, 90%, 91%, 92%, 93%, 94%, 95%, 96%, 97%, 98%, 99%, or 100% identical to the amino acid sequence of the second light chain outside of the VL region of the second light chain.

### 5.5.1 Exemplary Properties of CD161 Binding Proteins

### 5.5.1.1 Affinity of CD161 Binding Proteins for CD161

In some embodiments, the CD161 binding protein (e.g., an anti-CD161 antibody) (e.g., described herein)) exhibits a dissociation constant (Kd) for CD161 (e.g., hCD161) binding of <10μ, <100μ, <10μ, <1μ, <100 nM, <10 nM, <1 nM, <0.1 nM, <0.01 nM, or <0.001 nM, and/or >0.01 μM, 0.1 μM, or 1 μM (e.g., 10-5 M or less, 10-6 M or less, 10-8 M or less, e.g., from 1 μM to 10 μM, e.g., from 0.1 μM to 10 μM, e.g., from 10-6 M to 10-9 M, e.g., from 10-8 M to 10-13 M, e.g., from 10-9 M to 10-13 M) (e.g., as measured by SPR).

In some embodiments, the CD161 binding protein (e.g., an anti-CD161 antibody) (e.g., described herein)) exhibits a Kd for CD161 (e.g., hCD161) binding of <1 nM (e.g., as measured by SPR). In some embodiments, the CD161 binding protein (e.g., hCD161 binding protein) (e.g., described herein)) exhibits a Kd for CD161 (e.g., hCD161) binding of less than 1 nM (e.g., as measured by SPR). In some embodiments, the CD161 binding protein (e.g., hCD161 binding protein) (e.g., described herein)) exhibits a Kd for CD161 (e.g., hCD161) binding of from about 0.5 nM-1 nM (e.g., as measured by SPR).

In some embodiments, the CD161 binding protein (e.g., an anti-CD161 antibody) (e.g., described herein) exhibits a Kd for CD161 (e.g., hCD161) binding of <1 nM. In some embodiments, the CD161 binding protein (e.g., hCD161 binding protein) (e.g., described herein)) exhibits a Kd for CD161 (e.g., hCD161) binding of less than 1 nM. In some embodiments, the CD161 binding protein (e.g., an anti-CD161 antibody) (e.g., described herein)) exhibits a Kd for CD161 (e.g., hCD161) binding of from about 0.5 nM-1 nM.

Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip. In some embodiments, the affinity of a CD161 binding protein for CD161 (e.g., KD) is measured by SPR.

Other suitable assays for measuring the binding affinity include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.5.1.2 Inhibition of CD161 Binding to CLEC2D

In some embodiments, the CD161 binding protein (e.g., an anti-CD161 antibody) (e.g., described herein) inhibits (blocks) the binding of CD161 to CLEC2D. In some embodiments, the CD161 binding protein (e.g., an anti-CD161 antibody) (e.g., described herein) substantially inhibits (blocks) the binding of CD161 to CLEC2D. In some embodiments, the CD161 binding protein (e.g., an anti-CD161 antibody) (e.g., described herein) inhibits (blocks) detectable the binding of CD161 to CLEC2D. In some embodiments, the CD161 binding protein (e.g., an anti-CD161 antibody) (e.g., described herein) does not inhibit (block) the binding of CD161 to CLEC2D. In some embodiments, the CD161 binding protein (e.g., an anti-CD161 antibody) (e.g., described herein) does not substantially inhibit (block) the binding of CD161 to CLEC2D. In some embodiments, the CD161 binding protein (e.g., an anti-CD161 antibody) (e.g., described herein) does not inhibit (block) detectable the binding of CD161 to CLEC2D.

In some embodiments, the CD161 binding protein (e.g., hCD161 binding protein) (e.g., described herein)) inhibits (e.g., partially, substantially, or fully) binding of CD161 to CLEC2D. In some embodiments, the CD161 binding protein (e.g., hCD161 binding protein) (e.g., described herein)) partially inhibits binding of CD161 to CLEC2D. In some embodiments, the CD161 binding protein (e.g., hCD161 binding protein) (e.g., described herein)) substantially inhibits binding of CD161 to CLEC2D. In some embodiments, the CD161 binding protein (e.g., hCD161 binding protein) (e.g., described herein)) fully inhibits binding of CD161 to CLEC2D.

In some embodiments, the CD161 binding protein (e.g., hCD161 binding protein) (e.g., described herein)) inhibits (e.g., partially, substantially, or fully) binding of hCD161 to hCLEC2D. In some embodiments, the CD161 binding protein (e.g., hCD161 binding protein) (e.g., described herein)) partially inhibits binding of hCD161 to hCLEC2D. In some embodiments, the CD161 binding protein (e.g., hCD161 binding protein) (e.g., described herein)) substantially inhibits binding of hCD161 to hCLEC2D. In some embodiments, the CD161 binding protein (e.g., hCD161 binding protein) (e.g., described herein)) fully inhibits binding of hCD161 to hCLEC2D.

In some embodiments, the CD161 binding protein (e.g., hCD161 binding protein) (e.g., described herein)) inhibits (e.g., partially, substantially, or fully) binding of CD161 to CLEC2D and exhibits an IC50 of <10 μM, <100 μM, <10 μM, <1 μM, <100 nM, <10 nM, <1 nM, <0.1 nM, <0.01 nM, or <0.001 nM, and/or >0.01 μM, 0.1 μM, or 1 μM (e.g., 10-5M or less, 10-6 M or less, 10-8 M or less, e.g., from 1 μM to 10 μM, e.g., from 0.1 μM to 10 μM, e.g., from 10-6 M to 10-9 M, e.g., from 10-8 M to 10-13 M, e.g., from 10-9 M to 10-13 M) (e.g., as measured by SPR).

In some embodiments, the (e.g., hCD161 binding protein) (e.g., described herein)) inhibits (e.g., partially, substantially, or fully) binding of CD161 to CLEC2D and exhibits an IC50 of <10 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 5 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 2 nM. In some embodiments, the CD161 binding protein (e.g., hCD161 binding protein) (e.g., described herein)) exhibits a Kd for CD161 (e.g., hCD161) binding of from about 0.5 nM-2 nM.

In some embodiments, the CD161 binding protein (e.g., hCD161 binding protein) (e.g., described herein)) inhibits (e.g., partially, substantially, or fully) binding of CD161 to CLEC2D and exhibits an IC50 of <10 nM. In some embodiments, the CD161 binding protein (e.g., hCD161 binding protein) (e.g., described herein)) inhibits (e.g., partially, substantially, or fully) binding of CD161 to CLEC2D and exhibits an IC50 of less than 5 nM (e.g., as measured by SPR). In some embodiments, the CD161 binding protein (e.g., hCD161 binding protein) (e.g., described herein)) inhibits (e.g., partially, substantially, or fully) binding of CD161 to CLEC2D and exhibits an IC50 of less than 2 nM. In some embodiments, the CD161 binding protein (e.g., hCD161 binding protein) (e.g., described herein)) exhibits a Kd for CD161 (e.g., hCD161) binding of from about 0.5 nM-2 nM.

The inhibition (blocking) (or lack thereof) of CD161 binding to CLEC2D can be determined by standard methods known in the art. For example, an enzyme linked immunosorbent assays (ELISA). Standard binding assays to measure binding of CD161 to CLEC2D are known in the art and described herein. See, e.g., Wade M, Mendez J, Coussens N P, et al. Inhibition of Protein-Protein Interactions: Cell-Based Assays. 2017 Nov. 20. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK464632/; Arkin M R, Glicksman M A, Fu H, et al. Inhibition of Protein-Protein Interactions: Non-Cellular Assay Formats. 2012 Mar. 18 [Updated 2012 Oct. 1]. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK92000/; the entire contents of each of which are incorporated herein by reference for all purposes. For example, non-cell-based assays include, but are not limited to, ELISA. For further example, cell-based assays include, but are not limited to, energy transfer (Förster resonance energy transfer and bioluminescence resonance energy transfer) and protein complementation (fluorescence or enzymatic, e.g., luciferase).

### 5.5.1.3 Depletion of Populations of CD161 Expressing Cells and Cytokines

In some embodiments, the CD161 binding protein (e.g., the anti-CD161 antibody) is capable of mediating depletion of a population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, B cells, MAIT cells (Mucosal Associated Innate T cells), gamma delta T cells (gd T cells), Tregs (Regulatory T cells), and/or ILCs (innate lymphoid cells))) (e.g., upon binding to CD161 expressed on the surface of the population of cells). In some embodiments, the population of CD161 expressing cells comprises one or more subpopulations of immune cells. In some embodiments, the population of CD161 expressing cells comprises T cells (e.g., activated T cells) (e.g., CD4+ T cells and/or CD8+ T cells). In some embodiments, the population of CD161 expressing cells comprises CD4+ T cells and/or CD8+ T cells (e.g., activated CD4+ T cells and/or CD8+ T cells). In some embodiments, the population of CD161 expressing cells comprises Th2 cells, peTh2 cells, TH2A cells, and/or ILC2 cells. In some embodiments, the population of CD161 expressing cells comprises TH2A cells.

In some embodiments, the depletion of the population of CD161 expressing cells (e.g., immune cells (e.g., T cells (e.g., activated T cells) (e.g., CD4+ T cells, CD8+ T cells, Th2 cells, peTh2 cells, TH2A cells, ILC2 cells), NK cells, monocytes, B cells, MAIT cells (Mucosal Associated Innate T cells), gamma delta T cells (gd T cells), Tregs (Regulatory T cells), and/or ILCs (innate lymphoid cells))) mediates a reduction in the level of one or more proinflammatory cytokine (e.g., IFNγ, TNFα, IL-17, IL-22, IL-21, GM-CSF, IL-4, IL-13, IL-5 and IL-9). In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9.

In some embodiments, the population of CD161 expressing cells comprises CD8+ T cells and mediates a reduction in the level of one or more of IFNγ, TNFα, IL-17, IL-22, IL-21, or GM-CSF. In some embodiments, the population of CD161 expressing cells comprises TH2A cells and mediates a reduction in the level of one or more of IL-4, IL-13, IL-5 and IL-9.

In some embodiments, the CD161 binding protein (e.g., the anti-CD161 antibody) is capable of mediating a reduction in the level of one or more proinflammatory cytokine (e.g., IFNγ, TNFα, IL-17, IL-22, IL-21, GM-CSF, IL-4, IL-13, IL-5 or IL-9) (e.g., produced by a population of cells (e.g., immune cells (e.g., T cells (e.g., activated T cells) (e.g., CD4+ T cells, CD8+ T cells, Th2 cells, peTh2 cells, TH2A cells, ILC2 cells), NK cells, monocytes, B cells, MAIT cells (Mucosal Associated Innate T cells), gamma delta T cells (gd T cells), Tregs (Regulatory T cells), and/or ILCs (innate lymphoid cells)))). In some embodiments, the CD161 binding protein (e.g., the anti-CD161 antibody) is capable of mediating a reduction in the level of one or more proinflammatory cytokine (e.g., IFNγ, TNFα, IL-17, IL-22, IL-21, GM-CSF, IL-4, IL-13, IL-5 or IL-9) produced a population of cells (e.g., immune cells (e.g., T cells (e.g., activated T cells) (e.g., CD4+ T cells, CD8+ T cells, Th2 cells, peTh2 cells, TH2A cells, ILC2 cells), NK cells, monocytes, B cells, MAIT cells (Mucosal Associated Innate T cells), gamma delta T cells (gd T cells), Tregs (Regulatory T cells), and/or ILCs (innate lymphoid cells))). In some embodiments, the reduction in the level of one or more proinflammatory cytokine (e.g., IFNγ, TNFα, IL-17, IL-22, IL-21, GM-CSF, IL-4, IL-13, IL-5 or IL-9) is mediated by the depletion of the population of CD161 expressing cells (e.g., immune cells (e.g., T cells (e.g., CD4+ T cells, CD8+ T cells, Th2 cells, peTh2 cells, TH2A cells, ILC2 cells), NK cells, monocytes, B cells, MAIT cells (Mucosal Associated Innate T cells), gamma delta T cells (gd T cells), Tregs (Regulatory T cells), and/or ILCs (innate lymphoid cells))). In some embodiments, the population of CD161 expressing cells comprises one or more subpopulations of immune cells. In some embodiments, the population of CD161 expressing cells comprises T cells (e.g., activated T cells) (e.g., CD4+ T cells and/or CD8+ T cells). In some embodiments, the population of CD161 expressing cells comprises CD4+ T cells and/or CD8+ T cells (e.g., activated CD4+ T cells and/or CD8+ T cells). In some embodiments, the population of CD161 expressing cells comprises Th2 cells, peTh2 cells, TH2A cells, and/or ILC2 cells. In some embodiments, the one or more proinflammatory cytokine is IFNγ, TNFα, IL-17, IL-22, IL-21, or GM-CSF. In some embodiments, the one or more proinflammatory cytokine is IL-4, IL-13, IL-5 or IL-9. In some embodiments, the population of CD161 expressing cells comprises CD8+ T cells (e.g., activated CD8+ T cells) and the one or more proinflammatory cytokine is IFNγ, TNFα, IL-17, IL-22, IL-21, or GM-CSF. In some embodiments, the population of CD161 expressing cells comprises TH2A cells and the one or more proinflammatory cytokine is IL-4, IL-13, IL-5 or IL-9.

In some embodiments, the CD161 binding protein (e.g., an anti-CD161 antibody) mediates depletion of the population of cells through one or more hIg Fc effector function. In some embodiments, the CD161 binding protein (e.g., an anti-CD161 antibody) mediates depletion of the population of cells through any one or more of antibody dependent cellular cytotoxicity (ADCC), antibody dependent cellular phagocytosis (ADCP), or complement dependent cytotoxicity (CDC). In some embodiments, the CD161 binding protein (e.g., an anti-CD161 antibody) mediates depletion of the population of cells, at least in part, through ADCC.

### 5.6 Methods of Making Proteins

The CD161 binding proteins (e.g., anti-CD161 antibodies) (and polypeptides thereof) (and the fusion proteins thereof) described herein may be produced using standard methods known in the art.

For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the CD161 binding protein (or polypeptide or fragment thereof) (or the fusion protein). Such general methods are common knowledge in the art. The expression vector typically contains an expression cassette that includes nucleic acid sequences capable of bringing about expression of the nucleic acid molecule encoding the protein of interest (e.g., encoding the CD161 binding protein (or polypeptide thereof) (or the fusion protein)), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein of interest. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. see, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes. The CD161 binding proteins (and polypeptides thereof) (and fusion proteins) described herein may be produced synthetically.

As such, in one aspect, described herein is a method of manufacturing a CD161 binding protein (or a polypeptide thereof) described herein (or a fusion protein described herein), the method comprising introducing into a cell a nucleic acid molecule described herein (e.g., a nucleic acid molecule encoding a CD161 binding protein described herein, a fusion protein described herein), a vector described herein (e.g., a vector comprising a nucleic acid molecule described herein), or a carrier described herein (e.g., a carrier comprising a nucleic acid molecule or a vector described herein); culturing the cell under conditions that allow for expression of the CD161 binding protein (or polypeptide thereof) (or the fusion protein); and optionally recovering the expressed the CD161 binding protein (or polypeptide thereof) (or the fusion protein) from the culture; and optionally purifying the expressed the CD161 binding protein (or polypeptide thereof) (or the fusion protein) from the culture.

In some embodiments, the cell cannot mediate fucosylation of produced CD161 binding protein. Exemplary cells that cannot mediate fucosylation are known in the art. See, e.g., Pereira, Natasha A et al. “The “less-is-more” in therapeutic antibodies: Afucosylated anti-cancer antibodies with enhanced antibody-dependent cellular cytotoxicity.” mAbs vol. 10, 5 (2018):693-711. doi:10.1080/19420862.2018.1466767, the entire contents of which is incorporated herein by reference for all purposes.

In some embodiments, the disclosure features methods of making the CD161 binding proteins described herein (and fusion proteins described herein). The method includes (a) recombinantly expressing a CD161 binding protein (or a fusion protein comprising the same) described herein; (b) enriching, e.g., purifying, the CD161 binding protein (or the fusion protein comprising the same); (c) evaluating the CD161 binding protein (or the fusion protein comprising the same) described herein for the presence of a process impurity or contaminant, and (d) formulating the CD161 binding protein (or the fusion protein comprising the same) as a pharmaceutical composition if the CD161 binding protein (or the fusion protein comprising the same) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.6.1 Methods of Making Afucosylated Antibodies

As described herein, in specific preferred embodiments, the antibody is afucosylated. In one embodiments, the afucosylation is mediated through the expression of the antibody in a recombinant cell line with decreased or no expression of fucosyltransferase 8 (FUT8). The disruption of the FUT8 gene can be mediated, e.g., through the use of a CRISPR/Cas based system, a zinc finger nuclease (ZNF) based system, or a transcription activator-like effector nuclease based system (TALEN). For example, see, e.g., U.S. Pat. No. 8,679,491B2, U.S. Pat. No. 8,067,232B2, U.S. Pat. No. 8,313,913B2, U.S. Pat. No. 7,163,824B2, EP1504092B2, U.S. Pat. No. 9,145,565B2, U.S. Pat. No. 9,322,036B2, and WO2025140609A1, the entire contents of each of which is incorporated herein by reference for all purposes.

As such, in one aspect, provided herein are methods of manufacturing an afucosylated CD161 binding protein described herein, the method comprising introducing into a cell comprising a functional disruption in the FUT8 gene a nucleic acid molecule encoding a CD161 binding protein described herein; culturing the cell under conditions that allow for expression of the CD161 binding protein; and optionally recovering the expressed the CD161 binding protein from the culture; and optionally purifying the expressed the CD161 binding protein from the culture, wherein the CD161 binding protein expressed is afucosylated.

As such, in one aspect, provided herein are methods of manufacturing an afucosylated full length anti-CD161 antibody described herein, the method comprising introducing into a cell comprising a functional disruption in the FUT8 gene a nucleic acid molecule encoding the full length anti-CD161 antibody; culturing the cell under conditions that allow for expression of the anti-CD161 antibody; and optionally recovering the expressed the anti-CD161 antibody from the culture; and optionally purifying the expressed the anti-CD161 antibody from the culture, wherein the expressed anti-CD161 antibody is afucosylated.

### 5.7 Polynucleotides, Vectors, Carriers, & Cells

In one aspect, provided herein are nucleic acid molecules (e.g., DNA, RNA) encoding a CD161 binding protein (or a polypeptide thereof) described herein, a fusion protein described herein, or a conjugate described herein. In some embodiments, the nucleic acid molecule is a DNA molecule. In some embodiments, the nucleic acid molecule is an RNA nucleic acid molecule. In some embodiments, the nucleic acid molecule is an mRNA molecule.

In some embodiments, the nucleic acid molecule is a linear coding nucleic acid construct. In some embodiments, the nucleic acid molecule is contained within a vector non-viral vector (e.g., a plasmid), viral vector). In some embodiments, the nucleic acid molecule is contained within a non-viral vector (e.g., a plasmid). In some embodiments, the nucleic acid molecule is contained within a plasmid. In some embodiments, the nucleic acid molecule is contained within a viral vector.

In some embodiments, the nucleic acid molecule is modified (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the nucleic acid molecule is codon optimized. Codon optimization, may be used to match codon frequencies in target and host organisms to ensure proper folding; bias guanosine (G) and/or cytosine I content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In one aspect, provided herein are vectors comprising a nucleic acid molecule (e.g., DNA, RNA (e.g., mRNA)) described herein. In some embodiments, the vector is a viral vector. In some embodiments, the vector is a non-viral vector (e.g., a plasmid, minicircle). In some embodiments, the vector is a plasmid. In some embodiments, the vector is a minicircle.

In one aspect, provided herein a cell (e.g., a host cell, a therapeutic cell) or a population of cells (e.g., a population of host cells, a population of therapeutic cells) comprising a CD161 binding protein (or a polypeptide thereof) described herein, a fusion protein described herein, a conjugate described herein, a nucleic acid molecule described herein, or a vector described herein (e.g., a vector comprising a nucleic acid molecule described herein).

In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo.

Standard methods known in the art can be utilized to deliver any one of the foregoing (e.g., immunoreceptor targeting protein, fusion protein, vector, nucleic acid molecule, carrier, etc.) into a cell (e.g., a host cell). Standard methods known in the art can be utilized to culture cells (e.g., host cells) in vitro or ex vivo.

In some embodiments, the cell (or population of cells) expresses a protein comprising a CD161 binding protein described herein or a fusion protein described herein. In some embodiments, the cell (or population of cells) has been genetically engineered to comprise (e.g., within the cell's genome) a nucleic acid molecule (e.g., described herein) that encodes a CD161 binding protein described herein or a fusion protein described herein. In some embodiments, the cell (or population of cells) expresses a protein comprising a CD161 binding protein described herein or a fusion protein described herein on the surface of the cell.

In some embodiments, the cell (or population of cells) is a therapeutic cell. In some embodiments, the therapeutic cell (or population of cells) has been genetically engineered to comprise (e.g., within the cell's genome) a nucleic acid molecule (e.g., described herein) that encodes a CD161 binding protein described herein or a fusion protein described herein. In some embodiments, the therapeutic cell (or population of cells) expresses a protein comprising a CD161 binding protein described herein or a fusion protein described herein on the surface of the cell. In some embodiments, an immunoreceptor targeting protein described herein or a fusion protein described herein on the surface of the cell acts as a targeting moiety. In some embodiments, the therapeutic cell is an immune cell. In some embodiments, the therapeutic cell is a T cell (e.g., a CD8+ T cell, a CD4+ T cell). In some embodiments, the therapeutic cell is a natural killer cell.

In some embodiments, the cell express and/or encodes a chimeric antigen receptor comprising a CD161 binding protein (e.g., described herein) (also referred to herein as a CAR cell). As such, in one aspect, provided herein are cells expressing and/or genetically encoding a chimeric antigen receptor comprising a CD161 binding protein (e.g., utilized as the antigen binding domain of the extracellular domain of the chimeric antigen receptor). Exemplary chimeric antigen receptors are described herein (see, e.g., § 5.4.1). In some embodiments, the cell is a T cell (e.g., a CD8+ T cell, a CD4+ T cell). In some embodiments, the cell is a natural killer cell.

In one aspect, provided herein are carriers comprising a CD161 binding protein (or a polypeptide thereof) or polypeptide described herein, a fusion protein described herein, or a conjugate described herein, a nucleic acid molecule described herein, or a vector described herein (e.g., a vector comprising a nucleic acid molecule described herein). Exemplary carriers include, but are not limited to, nanoparticles, polymers, viruses (e.g., a recombinant viruses), virus like particles, virosomes, fusosomes, vesicles, or lipid-based carriers (e.g., lipid nanoparticles (LNPs), liposomes, lipoplexes, nanoliposomes, exosomes, or micelles). In some embodiments, the carrier is a lipid-based carrier such as an LNP, liposome, lipoplex, or nanoliposome. In some embodiments, the carrier is an LNP.

### 5.8 Pharmaceutical Compositions

In one aspect, provided herein are pharmaceutical compositions comprising a CD161 binding protein described herein, a fusion protein described herein, a conjugate described herein, a nucleic acid molecule described herein, a vector described herein, a cell (or population of cells) described herein, or a carrier described herein, and a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising a CD161 binding protein described herein, a fusion protein described herein, a conjugate described herein, a nucleic acid molecule described herein, a vector described herein, a cell (or population of cells) described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing a CD161 binding protein described herein, a fusion protein described herein, a conjugate, a nucleic acid molecule described herein, a vector described herein, a cell (or population of cells) described herein, or a carrier described herein, and formulating it into a pharmaceutically acceptable composition by the addition of one or more pharmaceutically acceptable excipient.

Acceptable excipients are preferably nontoxic to recipients at the dosages and concentrations employed, and include buffers such as phosphate, citrate, or other organic acids; antioxidants including or ascorbic acid methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein. Non-limiting examples include system administration or local administration. In specific embodiments, the pharmaceutical composition is administered intravenously. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.9 Methods of Use

Provided herein are, inter alia, various methods of utilizing a CD161 binding protein described herein (e.g., an anti-CD161 antibody) (as well as polynucleotides encoding the same, vectors encoding the same, cells (or populations of cells) comprising any of the foregoing, carriers comprising any of the foregoing, and pharmaceutical compositions comprising any of the foregoing).

As described below, in some aspects and embodiments, the methods comprise administration of a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein))), a cell (or population of cells) described herein (e.g., a cell (or population of cells) comprising a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein)))), or a carrier described herein (e.g., a carrier comprising a CD161 binding protein described herein (e.g., an anti-CD161 antibody described herein), a polynucleotide described herein (e.g., a polynucleotide encoding a CD161 binding protein described herein (e.g., an anti-CD161 antibody described herein)), a vector described herein (e.g., a vector comprising a polynucleotide described herein (e.g., a polynucleotide encoding a CD161 binding protein described herein (e.g., an anti-CD161 antibody described herein))), or a cell (or population of cells) described herein) to a subject.

Exemplary subjects include mammals, e.g., humans, non-human mammals, e.g., non-human primates. In preferred embodiments, the subject is a human. The dosage of any of the foregoing a CD161 binding protein described herein (e.g., an anti-CD161 antibody described herein) to be administered to a subject in accordance with any of the methods described herein can be determined in accordance with standard techniques known to those of ordinary skill in the art, including the route of administration, the age and weight of the subject, and the type (if any) adjuvant is used.

Exemplary routes of administering of an agent described herein (e.g., a CD161 binding protein described herein) include, e.g., systemic administration or local administration. In some embodiments, the route of administration is systemic. In some embodiments, the agent is administered intravenously, intramuscularly, or subcutaneously. In specific embodiments, the agent is administered intravenously. In some embodiments, the agent is administered intramuscularly. In specific embodiments, the agent is administered subcutaneously.

### 5.9.1 Methods of Delivery

Provided herein are methods of delivering (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein to a cell, the method comprising introducing into the cell the CD161 binding protein, the fusion protein, the conjugate, the nucleic acid molecule, the vector, the cell (or population of cells), the carrier, or the pharmaceutical composition to the subject, to thereby deliver the CD161 binding protein, the fusion protein, the conjugate, the nucleic acid molecule, the vector, the cell (or population of cells), the carrier, or the pharmaceutical composition, to the cell.

In specific preferred embodiments, the method comprises delivering (i) the CD161 binding protein described herein.

In some embodiments, the CD161 binding protein, the fusion protein, the conjugate, the nucleic acid molecule, the vector, the cell (or population of cells), the carrier, or the pharmaceutical composition is introduced in an amount and for a time sufficient to deliver the CD161 binding protein, the fusion protein, the conjugate, the nucleic acid molecule, the vector, the cell (or population of cells), the carrier, or the pharmaceutical composition to the cell. In some embodiments, the cell is in vitro, ex vivo, or in vivo. In some embodiments, the cell is in a subject (e.g., a human subject).

Provided herein are methods of delivering (i) a CD161 binding protein described herein; (ii) a fusion protein described herein; (iii) a conjugate described herein; (iv); a nucleic acid molecule described herein; (v) a vector described herein; (vi) a cell (or a population of cells) described herein; (vii) a carrier described herein; or (vii) a pharmaceutical composition described herein to a subject, the method comprising administering to the subject the CD161 binding protein, the fusion protein, the conjugate, the nucleic acid molecule, the vector, the cell (or population of cells), the carrier, or the pharmaceutical composition to the subject, to thereby deliver the CD161 binding protein, the fusion protein, the conjugate, the nucleic acid molecule, the vector, the cell (or population of cells), the carrier, or the pharmaceutical composition, to the subject.

In specific preferred embodiments, the method comprises delivering the CD161 binding protein described herein or a pharmaceutical composition comprising the same.

In some embodiments, the CD161 binding protein, the fusion protein, the conjugate, the nucleic acid molecule, the vector, the cell (or population of cells), the carrier, or the pharmaceutical composition is administered in an amount and for a time sufficient to deliver the CD161 binding protein, the fusion protein, the conjugate, the nucleic acid molecule, the vector, the cell (or population of cells), the carrier, or the pharmaceutical composition to the subject.

### 5.9.2 Methods of Inhibiting Binding of CD161 to CLEC2D

Provided herein are methods of inhibiting binding of CD161 to CLEC2D expressed on the surface of a cell in a subject, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby inhibit binding of CD161 to CLEC2D expressed on the surface of a cell in the subject. In some embodiments, the CD161 binding protein, the fusion protein, the conjugate, the nucleic acid molecule, the vector, the cell (or population of cells), the carrier, or the pharmaceutical composition is administered in an amount and for a time sufficient to inhibit binding of CD161 to CLEC2D expressed on the surface of a cell in the subject.

In specific preferred embodiments, the method comprises administering the CD161 binding protein described herein or a pharmaceutical composition comprising the same.

Standard binding assays to measure binding of CLEC2D and CD161 are known in the art and described herein. See, e.g., Wade M, Méndez J, Coussens N P, et al. Inhibition of Protein-Protein Interactions: Cell-Based Assays. 2017 Nov. 20. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK464632/; Arkin M R, Glicksman M A, Fu H, et al. Inhibition of Protein-Protein Interactions: Non-Cellular Assay Formats. 2012 Mar. 18 [Updated 2012 Oct. 1]. In: Markossian S, Grossman A, Brimacombe K, et al., editors. Assay Guidance Manual [Internet]. Bethesda (MD): Eli Lilly & Company and the National Center for Advancing Translational Sciences; 2004-. Available from: https://www.ncbi.nlm.nih.gov/books/NBK92000/; the entire contents of each of which are incorporated herein by reference for all purposes. For example, non-cell-based assays include, but are not limited to, ELISA. For further example, cell-based assays include, but are not limited to, energy transfer (Förster resonance energy transfer and bioluminescence resonance energy transfer) and protein complementation (fluorescence or enzymatic, e.g., luciferase).

### 5.9.3 Methods of Treating a Disease

Provided herein are methods of treating, ameliorating, or preventing a disease in a subject in need thereof, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby treat, ameliorate, or prevent the disease in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to treat, ameliorate, or prevent the CD161 associated disease in the subject.

In specific preferred embodiments, the method comprises administering the CD161 binding protein described herein or a pharmaceutical composition comprising the same.

Provided herein are CD161 binding proteins, fusion proteins, conjugates, nucleic acid molecules, vectors, cells, or pharmaceutical compositions for use in the treatment of a disease (e.g., cancer) in a subject in need thereof.

Provided herein are CD161 binding proteins or pharmaceutical compositions comprising the same for use in the treatment of a disease (e.g., cancer) in a subject in need thereof.

Provided herein are CD161 binding proteins, fusion proteins, conjugates, nucleic acid molecules, vectors, cells, or pharmaceutical compositions for use as a medicament.

Provided herein are CD161 binding proteins or pharmaceutical compositions comprising the same for use as a medicament.

Provided herein are uses of a CD161 binding protein described herein, fusion protein described herein, conjugate described herein, nucleic acid molecule described herein, vector described herein, cell described herein, or pharmaceutical composition described herein for the manufacture of a medicament for the treatment of a disease (e.g., a proinflammatory disease (e.g., autoimmune disease)) in a subject in need thereof.

Provided herein are uses of a CD161 binding protein described herein or pharmaceutical composition comprising the same for the manufacture of a medicament for the treatment of a disease (e.g., a proinflammatory disease (e.g., autoimmune disease)) in a subject in need thereof.

### 5.9.4 Methods of Treating a Proinflammatory Disease

In one aspect, provided herein are methods of treating, ameliorating, or preventing a proinflammatory disease (e.g., an autoimmune disease) in a subject in need thereof, the method comprising administering to the subject (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby treat, ameliorate, or prevent the proinflammatory disease (e.g., an autoimmune disease) in the subject.

In preferred embodiments, the method comprises administration of the CD161 binding protein described herein (e.g., anti-CD161 antibody (e.g., described herein)) or a pharmaceutical composition comprising the same.

In some embodiments, the proinflammatory disease (e.g., an autoimmune disease) is treated, ameliorated, or prevented, at least in part, through the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the proinflammatory disease (e.g., an autoimmune disease) is treated, ameliorated, or prevented through the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the CD161 binding protein, the fusion protein, the conjugate, the nucleic acid molecule, the vector, the cell (or population of cells), the carrier, or the pharmaceutical composition mediates the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the depletion of the population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)) mediates the reduction of the level of one or more proinflammatory cytokines (e.g., IFNγ, TNFα, IL-17, IL-22, IL-21, GM-CSF, IL-4, IL-13, IL-5 or IL-9). In some embodiments, the population of CD161 expressing cells comprises CD8+ T cells (e.g., activated CD8+ T cells) and the one or more proinflammatory cytokines is IFNγ, TNFα, IL-17, IL-22, IL-21, and/or GM-CSF. In some embodiments, the population of CD161 expressing cells comprises TH2A cells and the one or more proinflammatory cytokines is IL-4, IL-13, IL-5 or IL-9.

In some embodiments, the proinflammatory disease (e.g., an autoimmune disease) is treated, ameliorated, or prevented, at least in part, through a reduction of the level of one or more proinflammatory cytokines (e.g., IFNγ, TNFα, IL-17, IL-22, IL-21, GM-CSF, IL-4, IL-13, IL-5 or IL-9). In some embodiments, the proinflammatory disease (e.g., an autoimmune disease) is treated, ameliorated, or prevented through a reduction of the level of one or more proinflammatory cytokines (e.g., IFNγ, TNFα, IL-17, IL-22, IL-21, GM-CSF, IL-4, IL-13, IL-5 or IL-9). In some embodiments, the CD161 binding protein, the fusion protein, the conjugate, the nucleic acid molecule, the vector, the cell (or population of cells), the carrier, or the pharmaceutical composition mediates a reduction of the level of one or more proinflammatory cytokines (e.g., IFNγ, TNFα, IL-17, IL-22, IL-21, GM-CSF, IL-4, IL-13, IL-5 or IL-9).

In some embodiments, the reduction in the level of one or more proinflammatory cytokine (e.g., IFNγ, TNFα, IL-17, IL-22, IL-21, GM-CSF, IL-4, IL-13, IL-5 or IL-9) is mediated by the depletion of the population of CD161 expressing cells (e.g., activated immune cells) (e.g., immune cells (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells) and the one or more proinflammatory cytokines is IFNγ, TNFα, IL-17, IL-22, IL-21, and/or GM-CSF. In some embodiments, the population of CD161 expressing cells comprises TH2A cells and the one or more proinflammatory cytokines is IL-4, IL-13, IL-5 or IL-9.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) or a pharmaceutical composition comprising the same) is administered at a therapeutically effective dosing regimen. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in an amount and for a period of time sufficient to treat the proinflammatory disease (e.g., an autoimmune disease) in the subject.

In some embodiment, the proinflammatory disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis, or an idiopathic inflammatory myopathies (e.g., dermatomyositis, polymyositis, anti synthetase syndrome, inclusion-body myositis), sarcoidosis, enteropathic arthritis, or palmoplantar pustulosis. In some embodiments, the proinflammatory disease is an allergic disease (including, e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (including, e.g., eosinophilic esophagitis, hypereosinophilic syndrome, allergic rhinoconjunctivitis, an IgE mediated disease (including, e.g., bullous pemphigoid, lupus nephritis, pemphigus, autoimmune pancreatitis, and chronic spontaneous urticaria).

In some embodiments, the proinflammatory disease is an autoimmune disease. In some embodiment, the autoimmune disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis, an idiopathic inflammatory myopathies (e.g., dermatomyositis, polymyositis, anti synthetase syndrome, inclusion-body myositis), sarcoidosis, enteropathic arthritis, or palmoplantar pustulosis. In some embodiments, the autoimmune disease is an allergic disease (including, e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (including, e.g., eosinophilic esophagitis, hypereosinophilic syndrome, allergic rhinoconjunctivitis, an IgE mediated disease (including, e.g., bullous pemphigoid, lupus nephritis, pemphigus, autoimmune pancreatitis, and chronic spontaneous urticaria).

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in combination with one or more additional therapeutic agent. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered prior to, concomitant with, and/or after the administration of one or more additional therapeutic agent. In some embodiments, the one or more additional therapeutic agent is an anti-inflammatory agent.

In one aspect, provided herein is the use of (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (in preferred embodiments the CD161 binding protein described herein (e.g., anti-CD161 antibody (e.g., described herein)) or a pharmaceutical composition comprising the same) for the manufacture of a medicament for the treatment, amelioration, or prevention a proinflammatory disease (e.g., an autoimmune disease) in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in the treatment, amelioration, or prevention a proinflammatory disease (e.g., an autoimmune disease) in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of treating, ameliorating, or preventing a proinflammatory disease (e.g., an autoimmune disease) in a subject in need thereof, the method comprising administering to the subject the CD161 binding protein, the fusion protein, the conjugate, the nucleic acid molecule, the vector, the cell (or population of cells), the carrier, or the pharmaceutical composition.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of treating, ameliorating, or preventing a proinflammatory disease (e.g., an autoimmune disease) in a subject in need thereof, the method comprising administering to the subject the CD161 binding protein, the fusion protein, the conjugate, the nucleic acid molecule, the vector, the cell (or population of cells), the carrier, or the pharmaceutical composition, wherein the CD161 binding protein, the fusion protein, the conjugate, the nucleic acid molecule, the vector, the cell (or population of cells), the carrier, or the pharmaceutical composition mediates the depletion of CD161 expressing cells (e.g., immune cells (e.g., T cells (e.g., CD4+ T cells, CD8+ T cells), NK cells, B cells, MAIT cells, gd T cells, Tregs, and/or ILCs)).

### 5.9.5 Methods of Treating Autoimmune Diseases

In one aspect, provided herein are methods of treating, ameliorating, or preventing an autoimmune disease in a subject in need thereof, the method comprising administering to the subject (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby treat, ameliorate, or prevent the autoimmune disease in the subject.

In preferred embodiments, the method comprises administration of the CD161 binding protein described herein (e.g., anti-CD161 antibody (e.g., described herein)) or a pharmaceutical composition comprising the same.

In some embodiments, the autoimmune disease (e.g., an autoimmune disease) is treated, ameliorated, or prevented, at least in part, through the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the autoimmune disease (e.g., an autoimmune disease) is treated, ameliorated, or prevented through the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the CD161 binding protein, the fusion protein, the conjugate, the nucleic acid molecule, the vector, the cell (or population of cells), the carrier, or the pharmaceutical composition mediates the depletion of a population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells).

In some embodiments, the depletion of the population of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)) mediates the reduction of the level of one or more autoimmune cytokines (e.g., IFNγ, TNFα, IL-17, IL-22, IL-21, GM-CSF, IL-4, IL-13, IL-5 or IL-9). In some embodiments, the population of CD161 expressing cells comprises CD8+ T cells (e.g., activated CD8+ T cells) and the one or more autoimmune cytokines is IFNγ, TNFα, IL-17, IL-22, IL-21, and/or GM-CSF. In some embodiments, the population of CD161 expressing cells comprises TH2A cells and the one or more autoimmune cytokines is IL-4, IL-13, IL-5 or IL-9.

In some embodiments, the autoimmune disease (e.g., an autoimmune disease) is treated, ameliorated, or prevented, at least in part, through a reduction of the level of one or more autoimmune cytokines (e.g., IFNγ, TNFα, IL-17, IL-22, IL-21, GM-CSF, IL-4, IL-13, IL-5 or IL-9). In some embodiments, the autoimmune disease (e.g., an autoimmune disease) is treated, ameliorated, or prevented through a reduction of the level of one or more autoimmune cytokines (e.g., IFNγ, TNFα, IL-17, IL-22, IL-21, GM-CSF, IL-4, IL-13, IL-5 or IL-9). In some embodiments, the CD161 binding protein, the fusion protein, the conjugate, the nucleic acid molecule, the vector, the cell (or population of cells), the carrier, or the pharmaceutical composition mediates a reduction of the level of one or more autoimmune cytokines (e.g., IFNγ, TNFα, IL-17, IL-22, IL-21, GM-CSF, IL-4, IL-13, IL-5 or IL-9).

In some embodiments, the reduction in the level of one or more autoimmune cytokine (e.g., IFNγ, TNFα, IL-17, IL-22, IL-21, GM-CSF, IL-4, IL-13, IL-5 or IL-9) is mediated by the depletion of the population of CD161 expressing cells (e.g., activated immune cells) (e.g., immune cells (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)). In some embodiments, the population of CD161 expressing cells comprises CD8+ T cells (e.g., activated CD8+ T cells) and the one or more autoimmune cytokines is IFNγ, TNFα, IL-17, IL-22, IL-21, and/or GM-CSF. In some embodiments, the population of CD161 expressing cells comprises TH2A cells and the one or more autoimmune cytokines is IL-4, IL-13, IL-5 or IL-9.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered at a therapeutically effective dosing regimen. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in an amount and for a period of time sufficient to treat the autoimmune disease in the subject.

In some embodiment, the autoimmune disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease (e.g., Crohn's disease, ulcerative colitis), ankylosing spondylitis, non-radiographic axial spondyloarthritis, systemic lupus erythematosus, discoid lupus erythematosus, rheumatoid arthritis, an idiopathic inflammatory myopathies (e.g., dermatomyositis, polymyositis, anti synthetase syndrome, inclusion-body myositis), sarcoidosis, enteropathic arthritis, or palmoplantar pustulosis. In some embodiments, the autoimmune disease is an allergic disease (including, e.g., asthma, atopic dermatitis, allergic eosinophilic asthma, food allergy, chronic rhinosinusitis, chronic rhinosinusitis with nasal polyps (CRSwNP), an eosinophilic gastrointestinal disorder (including, e.g., eosinophilic esophagitis, hypereosinophilic syndrome, allergic rhinoconjunctivitis, an IgE mediated disease (including, e.g., bullous pemphigoid, lupus nephritis, pemphigus, autoimmune pancreatitis, and chronic spontaneous urticaria).

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in combination with one or more additional therapeutic agent. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered prior to, concomitant with, and/or after the administration of one or more additional therapeutic agent. In some embodiments, the one or more additional therapeutic agent is an anti-inflammatory agent.

In one aspect, provided herein is the use of (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for the manufacture of a medicament for the treatment, amelioration, or prevention a an autoimmune disease in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in the treatment, amelioration, or prevention an autoimmune disease in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of treating, ameliorating, or preventing an autoimmune disease in a subject in need thereof, the method comprising administering to the subject the CD161 binding protein, the fusion protein, the conjugate, the nucleic acid molecule, the vector, the cell (or population of cells), the carrier, or the pharmaceutical composition.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of treating, ameliorating, or preventing an autoimmune disease in a subject in need thereof, the method comprising administering to the subject the CD161 binding protein, the fusion protein, the conjugate, the nucleic acid molecule, the vector, the cell (or population of cells), the carrier, or the pharmaceutical composition, wherein the CD161 binding protein, the fusion protein, the conjugate, the nucleic acid molecule, the vector, the cell (or population of cells), the carrier, or the pharmaceutical composition mediates the depletion of CD161 expressing cells (e.g., immune cells (e.g., activated immune cells) (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)).

### 5.9.6 Methods of Depleting CD161 Expressing Cells

In one aspect, provided herein are methods of depleting a population of cells (e.g., immune cells (e.g., T cells, NK cells, B cells, Th2 cells, peTh2 cells, TH2A cells, ILC2 cells)) expressing CD161 (e.g., on the surface) in a subject in need thereof, the method comprising administering to the subject (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby deplete a population of cells (e.g., immune cells (e.g., activated immune cells) (e.g., T cells, NK cells, B cells, Th2 cells, peTh2 cells, TH2A cells, ILC2 cells)) expressing CD161 (e.g., on the surface) in the subject.

In preferred embodiments, the method comprises administration of the CD161 binding protein described herein (e.g., anti-CD161 antibody (e.g., described herein)) or a pharmaceutical composition comprising the same.

In some embodiments, the population of cells comprises immune cells. In some embodiments, the population of cells comprises activated immune cells.

In some embodiments, the population of cells comprises T cells (e.g., CD4+ T cells, CD8+ T cells, Th2 cells, peTh2 cells, TH2A cells, ILC2 cells), NK cells, MAIT cells, gd T cells, Tregs, and/or ILCs. In some embodiments, the population of cells comprises T cells (e.g., CD4+ T cells, CD8+ T cells). In some embodiments, the population of cells comprises CD4+ T cells. In some embodiments, the population of cells comprises CD8+ T cells. In some embodiments, the population of cells comprises CD4+ T cells and CD8+ T cells. In some embodiments, the population of cells comprises T cells (e.g., CD4+ T cells, CD8+ T cells, Th2 cells, peTh2 cells, TH2A cells, ILC2 cells) and NK cells. In some embodiments, the population of cells comprises CD4+ T cells and NK cells. In some embodiments, the population of cells comprises CD8+ T cells and NK cells. In some embodiments, the population of cells comprises CD4+ T cells, CD8+ T cells, and NK cells. In some embodiments, the population of cells comprises T cells (e.g., CD4+ T cells, CD8+ T cells, Th2 cells, peTh2 cells, TH2A cells, ILC2 cells), NK cells, MAIT cells, gd T cells, Tregs, and/or ILCs.

In some embodiments, the population of cells comprises activated T cells (e.g., activated CD4+ T cells, activated CD8+ T cells), activated NK cells. In some embodiments, the population of cells comprises activated T cells (e.g., activated CD4+ T cells, activated CD8+ T cells). In some embodiments, the population of cells comprises activated CD4+ T cells. In some embodiments, the population of cells comprises activated CD8+ T cells. In some embodiments, the population of cells comprises activated CD4+ T cells and activated CD8+ T cells. In some embodiments, the population of cells comprises activated T cells (e.g., CD4+ T cells, CD8+ T cells) and activated NK cells. In some embodiments, the population of cells comprises activated CD4+ T cells and activated NK cells. In some embodiments, the population of cells comprises activated CD8+ T cells and activated NK cells. In some embodiments, the population of cells comprises activated CD4+ T cells, activated CD8+ T cells, and NK cells. In some embodiments, the population of cells comprises activated T cells (e.g., activated CD4+ T cells, activated CD8+ T cells), activated NK cells.

In some embodiments, the population of cels compromises Th2 cells, peTh2 cells, TH2A cells, and/or ILC2 cells.

In some embodiments, the population of cells is depleted through one or more Ig Fc effector function (e.g., described herein), for example antibody dependent cellular cytotoxicity (ADCC), antibody dependent cellular phagocytosis (ADCP), complement dependent cytotoxicity (CDC), Fc receptor (e.g., Fcγ receptor) binding affinity, and C1q binding affinity. In some embodiments, the population of cells is depleted, at least in part, through ADCC. ADCC, CDC, ADCP, Fc receptor (e.g., Fcγ receptor) binding affinity, and C1q binding affinity can be measured through standard methods known in the art and also described herein.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in an amount and for a period of time sufficient to deplete a population of cells (e.g., immune cells (e.g., activated immune cells) (e.g., T cells, Th2 cells, peTh2 cells, TH2A cells, ILC2 cells, NK cells, B cells)) expressing CD161 (e.g., on the surface) in the subject.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in combination with one or more additional therapeutic agent. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered prior to, concomitant with, and/or after the administration of one or more additional therapeutic agent. In some embodiments, the one or more additional therapeutic agent is an anti-inflammatory agent.

In one aspect, provided herein is the use of (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for the manufacture of a medicament for the depletion a population of cells (e.g., immune cells (e.g., activated immune cells) (e.g., T cells, Th2 cells, peTh2 cells, TH2A cells, ILC2 cells, NK cells, B cells, MAIT cells, gd T cells, Tregs, and/or ILCs)) expressing CD161 (e.g., on the surface) in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in the depletion a population of cells (e.g., immune cells (e.g., activated immune cells) (e.g., T cells, Th2 cells, peTh2 cells, TH2A cells, ILC2 cells, NK cells, B cells, MAIT cells, gd T cells, Tregs, and/or ILCs)) expressing CD161 (e.g., on the surface) in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of depleting a population of cells (e.g., immune cells (e.g., activated immune cells) (e.g., T cells, Th2 cells, peTh2 cells, TH2A cells, ILC2 cells, NK cells, B cells)) expressing CD161 (e.g., on the surface) in a subject in need thereof, the method comprising administering to the subject the CD161 binding protein, the fusion protein, the conjugate, the nucleic acid molecule, the vector, the cell (or population of cells), the carrier, or the pharmaceutical composition.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of depleting a population of cells (e.g., immune cells (e.g., activated immune cells) (e.g., T cells, Th2 cells, peTh2 cells, TH2A cells, ILC2 cells, NK cells, B cells)) expressing CD161 (e.g., on the surface) in a subject in need thereof, the method comprising administering to the subject the CD161 binding protein, the fusion protein, the conjugate, the nucleic acid molecule, the vector, the cell (or population of cells), the carrier, or the pharmaceutical composition, wherein the CD161 binding protein, the fusion protein, the conjugate, the nucleic acid molecule, the vector, the cell (or population of cells), the carrier, or the pharmaceutical composition mediates the depletion of CD161 expressing cells (e.g., immune cells (e.g., activated immune cells) (e.g., T cells (e.g., CD4+ T cells, CD8+ T cells), NK cells, B cells, MAIT cells, gd T cells, Tregs, and/or ILCs)) through one or more Fc effector function (e.g., described herein), for example ADCC, CDC, ADCP, Fc receptor (e.g., Fcγ receptor) binding affinity, and C1q binding affinity.

### 5.9.7 Methods of Reducing the Level of Proinflammatory Cytokines

In one aspect, provided herein are methods of reducing the level of one or more proinflammatory cytokines in a subject in need thereof, the method comprising administering to the subject (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby reduce the level of one or more proinflammatory cytokines in the subject.

In preferred embodiments, the method comprises administration of the CD161 binding protein described herein (e.g., anti-CD161 antibody (e.g., described herein)) or a pharmaceutical composition comprising the same.

In some embodiments, the level of at least 1, 2, 3, 4, 5, 6, 7, 8, 9, or 10 proinflammatory cytokines is reduced (e.g., simultaneously). In some embodiments, the level of a plurality of proinflammatory cytokines is reduced (e.g., simultaneously). Proinflammatory cytokines include, but are not limited to, IFNγ, TNFα, IL-17, IL-22, IL-21, GM-CSF, IL-4, IL-13, IL-5, and IL-9.

In some embodiments, the one or more proinflammatory cytokine comprises any one or more of IFNγ, TNFα, IL-17, IL-22, IL-21, GM-CSF, IL-4, IL-13, IL-5, or IL-9. In some embodiments, the one or more proinflammatory cytokine comprises any one or more of IFNγ, TNFα, IL-17, IL-22, IL-21, or GM-CSF. In some embodiments, the one or more proinflammatory cytokine comprises any one or more of IL-17, IL-22, IL-21, and GM-CSF. In some embodiments, the one or more proinflammatory cytokine comprises IL-4, IL-13, IL-5, or IL-9. In some embodiments, the one or more proinflammatory cytokine comprises any one or more of IFNγ, TNFα, IL-17, IL-22, IL-21, and GM-CSF.

In some embodiments, the one or more proinflammatory cytokine comprises IFNγ. In some embodiments, the one or more proinflammatory cytokine comprises TNFα. In some embodiments, the one or more proinflammatory cytokine comprises any one or more of IFNγ, and TNFα. In some embodiments, the one or more proinflammatory cytokine comprises IL-17. In some embodiments, the one or more proinflammatory cytokine comprises IL-22. In some embodiments, the one or more proinflammatory cytokine comprises IL-21. In some embodiments, the one or more proinflammatory cytokine comprises GM-CSF. In some embodiments, the one or more proinflammatory cytokine comprises IL-4. In some embodiments, the one or more proinflammatory cytokine comprises IL-13. In some embodiments, the one or more proinflammatory cytokine comprises IL-5. In some embodiments, the one or more proinflammatory cytokine comprises IL-9.

In some embodiments, the one more proinflammatory cytokines are expressed by one or more cells that express CD161. In some embodiments, the one or more cells are immune cells. In some embodiments, the one or more cells are T cells (e.g., CD4+ T cells, CD8+ T cells, Th2 cells, peTh2 cells, TH2A cells, ILC2 cells), NK cells, MAIT cells, gd T cells, Tregs, and/or ILCs. In some embodiments, the one or more cells are T cells (e.g., CD4+ T cells, CD8+ T cells, Th2 cells, peTh2 cells, TH2A cells, ILC2 cells). In some embodiments, the one or more cells are CD4+ T cells. In some embodiments, the one or more cells are CD8+ T cells. In some embodiments, the one or more cells are CD4+ T cells and CD8+ T cells. In some embodiments, the one or more cells are T cells (e.g., CD4+ T cells, CD8+ T cells) and NK cells. In some embodiments, the one or more cells are CD4+ T cells and NK cells. In some embodiments, the one or more cells are CD8+ T cells and NK cells. In some embodiments, the one or more cells are CD4+ T cells, CD8+ T cells, and NK cells. In some embodiments, the one or more cells are T cells (e.g., CD4+ T cells, CD8+ T cells), NK cells, MAIT cells, gd T cells, Tregs, and/or ILCs. In some embodiments, the one or more cells are, Th2 cells, peTh2 cells, TH2A cells, ILC2 cells.

In some embodiments, the reduction in the level of the one or more proinflammatory cytokines is mediated through the depletion of a population of cells (e.g., immune cells (e.g., T cells, Th2 cells, peTh2 cells, TH2A cells, ILC2 cells, NK cells, B cells, MAIT cells, gd T cells, Tregs, and/or ILCs)) expressing CD161 (e.g., on the surface). In some embodiments, the population of cells comprises T cells (e.g., CD4+ T cells, CD8+ T cells, Th2 cells, peTh2 cells, TH2A cells, ILC2 cells), NK cells, MAIT cells, gd T cells, Tregs, and/or ILCs. In some embodiments, the population of cells comprises T cells (e.g., CD4+ T cells, CD8+ T cells). In some embodiments, the population of cells comprises CD4+ T cells. In some embodiments, the population of cells comprises CD8+ T cells. In some embodiments, the population of cells comprises CD4+ T cells and CD8+ T cells. In some embodiments, the population of cells comprises T cells (e.g., CD4+ T cells, CD8+ T cells, Th2 cells, peTh2 cells, TH2A cells, ILC2 cells) and NK cells. In some embodiments, the population of cells comprises CD4+ T cells and NK cells. In some embodiments, the population of cells comprises CD8+ T cells and NK cells. In some embodiments, the population of cells comprises CD4+ T cells, CD8+ T cells, and NK cells. In some embodiments, the population of cells comprises T cells (e.g., CD4+ T cells, CD8+ T cells, Th2 cells, peTh2 cells, TH2A cells, ILC2 cells), NK cells, MAIT cells, gd T cells, Tregs, and/or ILCs. In some embodiments, the population of cells comprises Th2 cells, peTh2 cells, TH2A cells, and/or ILC2 cells.

In some embodiments, the population of cells is depleted through one or more Fc effector function (e.g., described herein), for example ADCC, CDC, ADCP, Fc receptor (e.g., Fcγ receptor) binding affinity, and C1q binding affinity. In some embodiments, the population of cells is depleted, at least in part, through ADCC. ADCC, CDC, ADCP, Fc receptor (e.g., Fcγ receptor) binding affinity, and C1q binding affinity can be measured through standard methods known in the art and also described herein.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in an amount and for a period of time sufficient to reduce the level of one or more proinflammatory cytokines in the subject.

In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered in combination with one or more additional therapeutic agent. In some embodiments, the CD161 binding protein, the fusion protein, the conjugate, the nucleic acid molecule, the vector, the cell (or population of cells), the carrier, or the pharmaceutical composition (e.g., the CD161 binding protein described herein (e.g., anti-CD161 antibody (e.g., described herein)) or a pharmaceutical composition comprising the same) is administered prior to, concomitant with, and/or after the administration of one or more additional therapeutic agent. In some embodiments, the one or more additional therapeutic agent is an anti-inflammatory agent.

In one aspect, provided herein is the use of (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for the manufacture of a medicament for the reduction of the level of one or more proinflammatory cytokines in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in the reduction of the level of one or more proinflammatory cytokines in a subject in need thereof.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of the reducing of the level of one or more proinflammatory cytokines in a subject in need thereof, the method comprising administering to the subject the CD161 binding protein, the fusion protein, the conjugate, the nucleic acid molecule, the vector, the cell (or population of cells), the carrier, or the pharmaceutical composition.

In one aspect, provided herein is (i) a CD161 binding protein described herein (e.g., anti-CD161 antibody (e.g., described herein)); (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein (e.g., anti-CD161 antibody (e.g., described herein)) or a pharmaceutical composition comprising the same) for use in a method of the reducing of the level of one or more proinflammatory cytokines in a subject in need thereof, the method comprising administering to the subject the CD161 binding protein, the fusion protein, the conjugate, the nucleic acid molecule, the vector, the cell (or population of cells), the carrier, or the pharmaceutical composition, wherein the CD161 binding protein, the fusion protein, the conjugate, the nucleic acid molecule, the vector, the cell (or population of cells), the carrier, or the pharmaceutical composition mediates the reduction of the one or more proinflammatory cytokines through the depletion of CD161 expressing cells (e.g., immune cells (e.g., T cells (e.g., CD4+ T cells, CD8+ T cells, Th2 cells, peTh2 cells, TH2A cells, ILC2 cells), NK cells, B cells, MAIT cells, gd T cells, Tregs, and/or ILCs)) (e.g., through one or more Fc effector function (e.g., described herein), for example ADCC, CDC, ADCP, Fc receptor (e.g., Fcγ receptor) binding affinity, and C1q binding affinity).

### 5.9.8 Methods of Treating Cancer

Provided herein are methods treating, ameliorating, or preventing cancer in a subject in need thereof, the method comprising administering to the subject (i) a CD161 binding protein described herein; (ii) a fusion protein described herein; (iii) a conjugate described herein; (iv); a nucleic acid molecule described herein; (v) a vector described herein; (vi) a cell (or population of cells) described herein; (vii) a carrier described herein; or (vii) a pharmaceutical composition described herein, to thereby treat, ameliorate, or prevent the cancer in the subject. In some embodiments, the CD161 binding protein, the fusion protein, the conjugate, the nucleic acid molecule, the vector, the cell (or population of cells), the carrier, or the pharmaceutical composition is administered to the subject in an amount and for a time sufficient to treat, ameliorate, or prevent the cancer in the subject.

In some embodiments, the cancer is a solid tumor. In some embodiments, the cancer is a carcinoma, adenocarcinoma, or sarcoma. In some embodiments, the cancer is a carcinoma, adenocarcinoma, or sarcoma. In some embodiments, the cancer is a carcinoma. In some embodiments, the cancer is an adenocarcinoma.

In some embodiments, the cancer is head cancer, neck cancer, head and neck cancer, lung cancer, colon cancer, rectal cancer, colorectal cancer, renal cancer, ovarian cancer, breast cancer, endometrial cancer, uterine cancer, cervical cancer, anal cancer, prostate cancer, bladder cancer, liver cancer, pancreatic cancer, thyroid cancer, thymus cancer, bronchus cancer, skin cancer, brain cancer, spinal cord cancer, lip cancer, bowel cancer (e.g., small bowel cancer, large bowel cancer), or oral cavity cancer. In some embodiments, the cancer is head cancer, neck cancer, head and neck cancer, lung cancer, colon cancer, rectal cancer, colorectal cancer, renal cancer, or ovarian cancer. In some embodiments, the cancer is head and neck squamous cell carcinoma (HNSCC), non-small cell lung cancer, colorectal carcinoma, renal cell carcinoma, cutaneous squamous cell carcinoma. In some embodiments, the cancer is non-small cell lung cancer, head and neck squamous cell carcinoma, triple negative breast cancer, cutaneous squamous cell carcinoma, hormone receptor positive breast carcinoma, small bowel cancer, esophageal cancer, or colorectal cancer.

In some embodiments, the cancer is head cancer, neck cancer, head and neck cancer, lung cancer, colon cancer, rectal cancer, colorectal cancer, renal cancer, ovarian cancer, breast cancer, endometrial cancer, uterine cancer, cervical cancer, anal cancer, prostate cancer, bladder cancer, liver cancer, pancreatic cancer, thyroid cancer, thymus cancer, bronchus cancer, skin cancer, brain cancer, spinal cord cancer, lip cancer, bowel cancer (e.g., small bowel cancer, large bowel cancer), or oral cavity cancer.

In some embodiments, the cancer is head cancer, neck cancer, head and neck cancer, lung cancer, colon cancer, rectal cancer, colorectal cancer, renal cancer, or ovarian cancer.

In some embodiments, the cancer is head and neck squamous cell carcinoma (HNSCC), non-small cell lung cancer, colorectal carcinoma, renal cell carcinoma, cutaneous squamous cell carcinoma.

In some embodiments, the cancer is non-small cell lung cancer, head and neck squamous cell carcinoma, triple negative breast cancer, cutaneous squamous cell carcinoma, hormone receptor positive breast carcinoma, small bowel cancer, esophageal cancer, or colorectal cancer.

In some embodiments, the cancer is a hematological cancer. In some embodiments, the cancer is a lymphoma, leukemia, or myeloma. In some embodiments, the cancer is a lymphoma. In some embodiments, the cancer is diffuse large B cell lymphoma, Hodgkin lymphoma, Burkitt lymphoma, or T-cell lymphoma.

In some embodiments, the cancer is a hematological cancer. In some embodiments, the cancer is a lymphoma, leukemia, or myeloma. In some embodiments, the cancer is a lymphoma. In some embodiments, the cancer is diffuse large B cell lymphoma, Hodgkin lymphoma, Burkitt lymphoma, or T-cell lymphoma. In some embodiments, the cancer is a leukemia (e.g., acute leukemia, acute lymphoblastic, leukemia (ALL) (e.g., B-cell ALL, T-cell ALL), acute myeloid leukemia (AML), chronic myelocytic leukemia (CML), chronic lymphocytic leukemia (CLL), hairy cell leukemia.

In specific embodiments, the cancer is a T cell leukemia, NK cell leukemia, T cell prolymphocytic leukemia (T-PLL), or large granular lymphocytic leukemia (LGLL).

In specific embodiments, the cancer is NK/T cell lymphoma (NKTCL), extranodal NK/T cell lymphoma (ENKL), mycosis fungoides (MF), Sezary syndrome, peripheral T cell lymphoma, angioimmunoblastic T cell lymphoma (AITL), and peripheral T cell lymphoma not otherwise specified (PTCL-NOS).

### 5.10 Kits

In a one aspect, provided herein are kits (e.g., diagnostic kits, therapeutic kits) comprising any one or more of a CD161 binding protein described herein; a fusion protein described herein; a conjugate described herein; a nucleic acid molecule described herein; a vector described herein; a cell (or population of cells) described herein; a carrier described herein; and/or a pharmaceutical composition described herein. In addition, the kit may comprise a liquid vehicle for solubilizing or diluting, and/or technical instructions. The technical instructions of the kit may contain information about administration and dosage and subject groups.

In some embodiments, the CD161 binding protein described herein, the fusion protein described herein, the conjugate described herein, the nucleic acid molecule described herein, the vector described herein, the cell (or population of cells) described herein, the carrier described herein, or the pharmaceutical composition described herein is provided in a separate part of the kit, wherein the CD161 binding protein described herein, the fusion protein described herein, the conjugate described herein, the nucleic acid molecule described herein, the vector described herein, the cell (or population of cells) described herein, the carrier described herein, or the pharmaceutical composition described herein is optionally lyophilized, spray-dried, or spray-freeze dried. The kit may further contain as a part a vehicle (e.g., buffer solution) for solubilizing the dried or lyophilized a CD161 binding protein described herein; a fusion protein described herein; a conjugate described herein; a nucleic acid molecule described herein; a vector described herein; a cell (or population of cells) described herein; a carrier described herein; and/or a pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

In some embodiments, the kit comprises a CD161 binding protein described herein; a fusion protein described herein; or a conjugate described herein. In some embodiments, CD161 binding protein described herein, the fusion protein described herein, or the conjugate described herein comprises a detectable label (e.g., a tag (e.g., a fluorescent tag)) to aid in detection. In some embodiments, the kit comprises one or more reagent (e.g., a buffer) for a sample described herein. In some embodiments, the kit comprises a secondary antibody comprising a detectable label that specifically binds the CD161 binding protein described herein, the fusion protein described herein, or the conjugate described herein. In some embodiments, the kit is for use in a method of determining the presence of a population of CD161-expressing cells in a sample. In some embodiments, the kit comprises a packaged combination of reagents in predetermined amounts with instructions for performing the diagnostic or detection assay. In some embodiments, the kit comprises a reagent or set of reagents for detecting a complex of CD161 bound to a CD161 binding protein described herein; a fusion protein described herein; or a conjugate described herein.

Any of the kits described herein may be used in any of the methods described herein (see, e.g., § 5.9).

### 6. EXAMPLES

TABLE OF CONTENTS

| | |
|---|---|
| 6.1 | Example 1. Generation and In Vitro Functional Characterization of Anti-CD161 |
| | Antibody Ab-1. |
| 6.2 | Example 2. Anti-CD161 Antibody Ab-1 Induction of Antibody Dependent Cellular |
| | Cytotoxicity (ADCC). |
| 6.3 | Example 3. In Vivo Assessment of Anti-CD161 Antibody Ab-1 in the Treatment of |
| | Skin Inflammation. |
| 6.4 | Example 4. In Vivo Assessment of Anti-CD161 Antibody Ab-1 in the Treatment of |
| | Skin Inflammation. |
| 6.5 | Example 5. In vitro CD161 Cell Depletion Reduces Multiple Inflammatory |
| | Mediators in Tissue Samples from Crohn's Disease Patients. |
| 6.6 | Example 6. Comparison of Ab-1 to Anti-CD161 Antibody HP-3G10. |
| 6.7 | Example 7. Afucosylated Ab-1 Induction of Antibody Dependent Cellular |
| | Phagocytosis (ADCP). |
| 6.8 | Example 8. Afucosylated Ab-1 Does Not Induce Cytokine Release Associated with |
| | Cytokine Release Syndrome (CRS). |
| 6.9 | Example 9. CD161 Cell Depletion Mediates Enhanced Reduction in Multiple |
| | Inflammatory Mediators in Colon Tissue Samples from Crohn's Disease Patients |
| | Compared to Other Therapeutics. |
| 6.1 | Example 1. Generation and In Vitro Functional Characterization of Anti-CD161 |
| | Antibody Ab-1. |

A novel antibody (referred to herein as Ab-1) that specifically binds CD161 was made. Briefly, eight naive human synthetic yeast libraries each of ˜10⁹ diversity were propagated and selected using human CD161 as the antigen, first by magnetic-activated cell sorting (MACS), followed by FACS sorting. Heavy chains from naive output were used for light chain batch shuffle. MACS and FACS sortings were performed to obtain populations with the desired characteristics. Individual colonies from each terminal FACS selection round were picked for sequencing and characterization. For additional affinity maturation, CDRs of both light and heavy chains were diversified in combinations. More stringent conditions were used to sort for clones with improved affinities. The selected VL/VH sequences were cloned into expression vectors with light chain constant region and heavy chain constant region, respectively; and transfected into CHO cells. After 7 days, the supernatants were harvested and purified with Protein A column. To produce afucosylated antibodies, a CHO cell line with FUT8 gene knockout was utilized.

The amino acid sequence is set forth in Table 2 (Ab-1) (SEQ ID NOS: 67-68). Ab-1 was functionally characterized in vitro for (i) human CD161 binding, (ii) cynomolgus macaque CD161 binding, and (iii) blocking of binding of CD161 to CLEC2D.

### 6.1.1 Anti-CD161 Antibody Ab-1 Binding to CD161.

In vitro binding of Ab-1 (SEQ ID NOS: 67-68) to recombinant human CD161 (hCD161) (His-tagged) and cynomolgus macaque CD161 (cynoCD161) (mono-Fc fusion) was assessed using a standard Octet binding assay.

Briefly, the AHC or FAB2G sensors were hydrated with kinetic buffer for 10 minutes at room temperature. After a 120 second baseline step, the sensors were dipped into 20 nM monoclonal antibodies, followed by a 30 second baseline step, then dipped into 100 nM of hCD161-His or monoFc-cynoCD161 for 180 sec (association), and into buffer for 300 second (dissociation). Kinetic data was analyzed and fitted using the 1:1 binding model.

The binding affinity of Ab-1 is set forth below in Table 7.

TABLE 7
Binding Affinity of Ab-1 to hCD161 and cynoCD161.

| | hCD161-His | | | monoFc-cyno CD161 | | |
|---|---|---|---|---|---|---|
| Sample | KD (M) | kon(1/Ms) | kdis(1/s) | KD (M) | kon(1/Ms) | kdis(1/s) |
| Ab-1 | 8.02E−10 | 2.77E+05 | 2.22E−04 | 2.40E−09 | 2.03E+05 | 4.85E−04 |

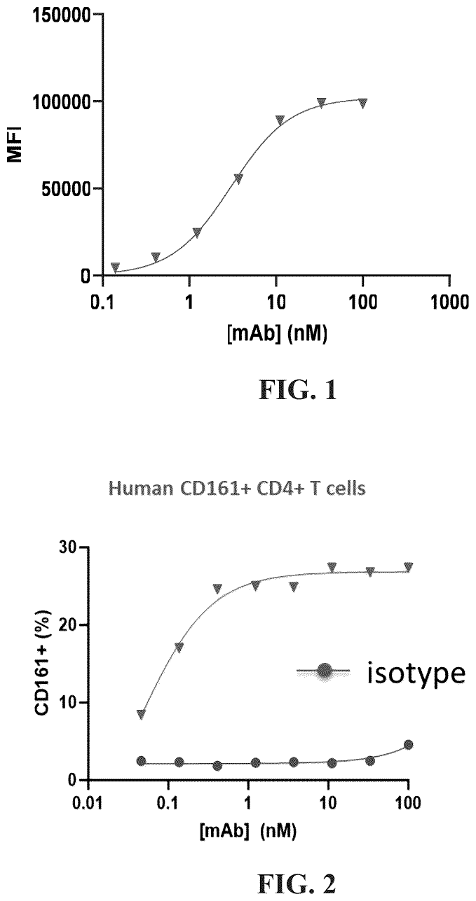

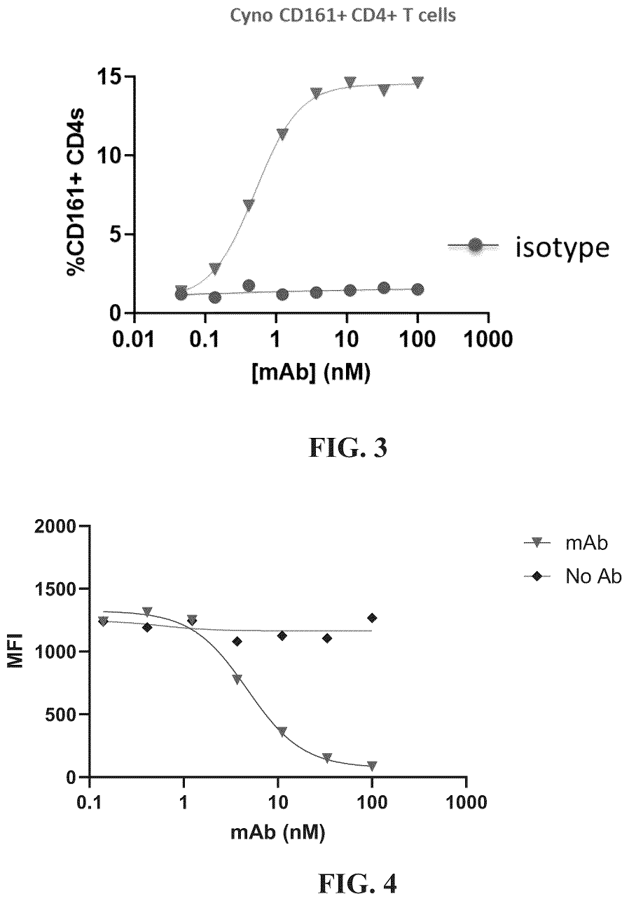

The ability of Ab-1 to bind to bind both HEK cells expressing CD161 and endogenous hCD161 and cynoCD161 expressed by CD4+ T cells in vitro was assessed. As shown in FIGS. 1-3, Ab-1 specifically bound hCD161 expressed on the surface of HEK cells (FIG. 1) and endogenous hCD161 (FIG. 2) and endogenous cynoCD161 (FIG. 3) expressed by CD4+ T cells.

### 6.1.2 Anti-CD161 Antibody Ab-1 Blocking of CLEC2D-CD161 Binding.

The ability of Ab-1 to block binding of CLEC2D to CD161 was also assessed. Briefly, Ab-1 was 3-fold serially diluted starting from 300 nM. 50 μL of each dilution was transferred to a 96-well V-bottom plate containing 50 μL/well of 300 nM biotinylated CLEC2D-Fc. Subsequently, 50 μL/well of CD161 overexpressing cells suspended at 1e6/mL was added to the plate, resulting a total of volume of 150 uL per well containing 50,000 cells, a constant concentration of 100 nM biotinylated CLEC2D-Fc, and Ab-1 concentration series from 100 nM to 0.14 nM. The plate was incubated at 4° C. for 1 hr, washed twice, and stained with 100 μL/well of 1:1000 Neutravidin-DyLight 650 (Thermo Fisher Scientific). After another 30 min incubation at 4° C. and two washes, the plate was analyzed on Attune flow cytometer. The median fluorescence intensity was plotted against the Ab-1 concentration. As shown in FIG. 4, Ab-1 blocked binding of CD161 to CLEC2D.

### 6.2 Example 2. Anti-CD161 Antibody Ab-1 Induction of Antibody Dependent Cellular Cytotoxicity (ADCC)

The ability of both a fucosylated and afucosylated Ab-1 to induce ADCC mediated killing of CD161 expressing cells (multiple T cell populations) was assessed by standard methods known in the art and summarized below.

### 6.2.1 Anti-CD161 Antibody Ab-1 Induction of ADCC Mediated Killing of Human T Cell Populations.

The ability of Ab-1 to induce ADCC mediated killing of CD161 expressing human T populations was assessed.

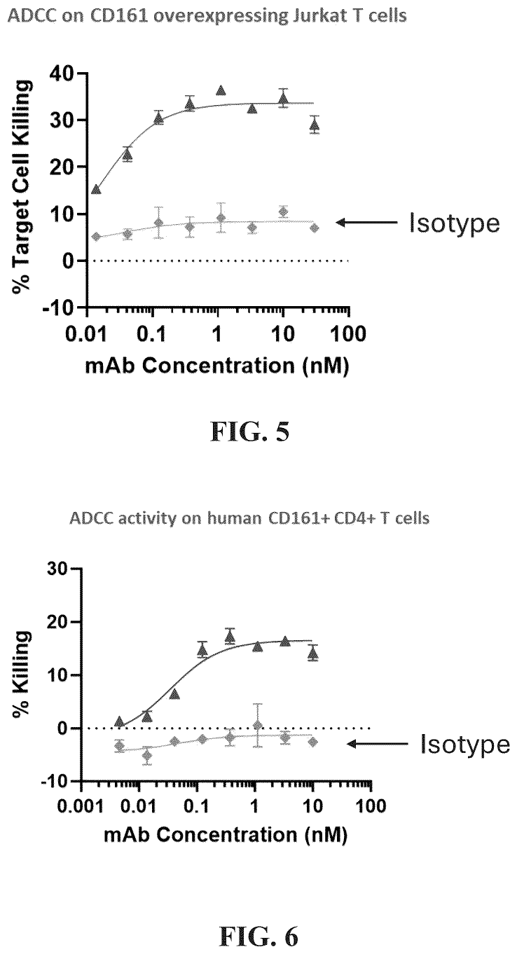

Briefly, NK cells were isolated from donor human PBMCs using Magnetic activated cell sorting (MACS) following vendor instructions. For the experiments presented in FIG. 5, Jurkat cells overexpressing CD161 were used as target cells and were plated at 50,000 cells/well of a 96-well plate. Ab1 was 3-fold serially diluted starting at 30 nM. For the experiments presented in FIG. 6, human PBMCs were used as target cells and were plated at 150,000 cells/well of a 96-well plate. Ab-1 was 3-fold serially diluted starting at 10 nM. Antibody dilutions were added to target cells and incubated on ice for 20 minutes to facilitate antibody binding. Next, freshly purified NK cells from the same donor as target PBMCs were added at 50,000 cells/well. The co-cultures were incubated for 4 h at 37° C. Post co-culture, the cells were stained with a viability dye (LIVE/DEAD™ Fixable Near IR) and surface markers (for FIG. 5: CD3, CD56; and for FIG. 6: CD3, CD4, CD56, CD161) for gating followed by analyses by flow cytometry. The % killing for each target population was calculated based on change in viability compared to control wells to which antibody was not added.

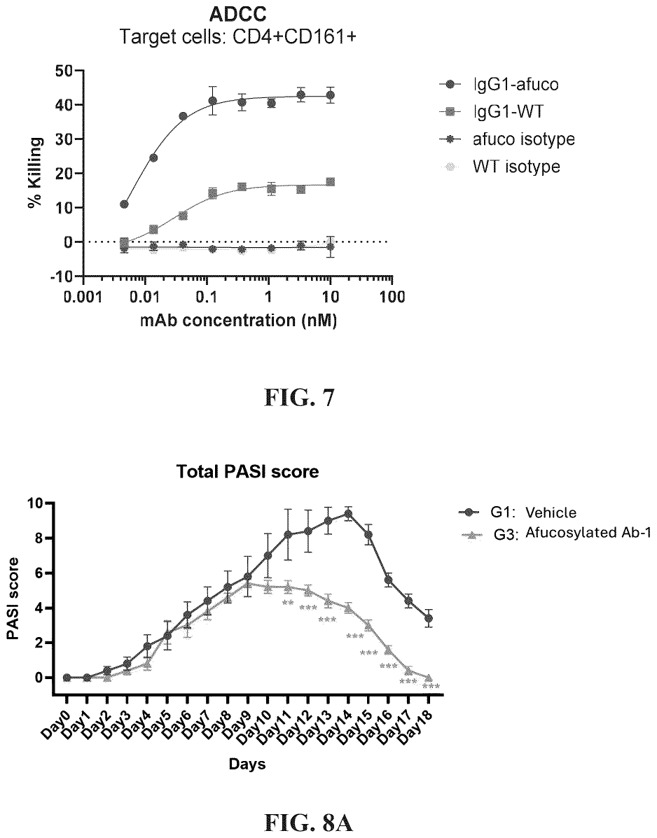

As shown in FIGS. 5-6, Ab-1 induced ADCC mediated killing of both CD161 expressing Jurkat cells (FIG. 5) and human T cells (FIG. 6) at various concentrations. As shown in FIG. 7, afucosylated Ab-1 showing enhanced ADCC potency compared to fucosylated Ab-1.

### 6.2.2 Example 2. Anti-CD161 Antibody Ab-1 Induction of ADCC Mediated Killing of Cyno T Cell Populations.

The ability of afucosylated Ab-1 to induce ADCC mediated killing of CD161 expressing cyno T populations was assessed.

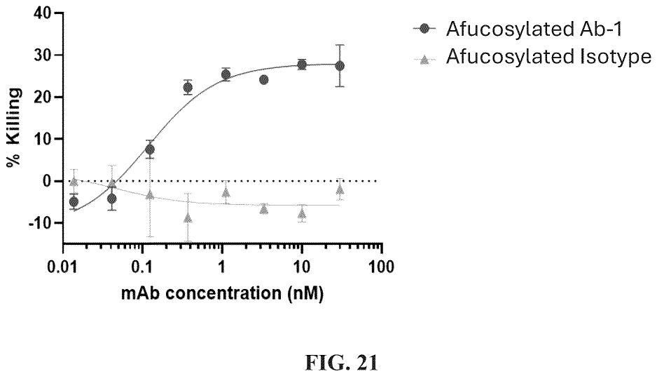

Briefly, PBMCs from Cynomolgus donors were plated at 150,000 cells/well of a 96-well plate. Ab-1 was 3-fold serially diluted starting at 30 nM. Antibody dilutions were added to PBMCs and incubated on ice for 20 minutes to facilitate antibody binding. The cultures were incubated for 4 h at 37° C. Post co-culture, the cells were stained with a viability dye (LIVE/DEAD™ Fixable Near IR) and surface markers (FIG. 21: CD3, CD8, CD161) for gating followed by analyses by flow cytometry. The % killing for each target population was calculated based on change in live cell count compared to control wells to which antibody was not added.

As shown in FIG. 21, afucosylated Ab-1 induced ADCC mediated killing of CD161 expressing cyno CD8+ T cells at various concentrations.

### 6.3 Example 3. In Vivo Assessment of Anti-CD161 Antibody Ab-1 in the Treatment of Skin Inflammation

The ability of afucosylated Ab-1 to treat skin inflammation in vivo was assessed. Briefly, an imiquimod induced skin inflammation model in cynomolgus macaque was utilized according to the study design set forth in Table 8 and Table 9 below. Generally daily imiquimod was administered on days 0-14; and inflammation resolution phase were days 15-29.

TABLE 8
Study Design in Imiquimod Induced Skin Inflammation Model in Cyno.

| | # of | | | | | | |
|---|---|---|---|---|---|---|---|
| Conditions | animals | Type | Dose | | Dose date | Route | Readouts |
| Vehicle | 5 | Naïve | N/A | | N/A | N/A | PASI Score |
| Anti IL17 | 5 | Naïve | 3 | mg/kg | Day 0, Day 7 | i.v. | Bosy Weight |
| Antibody | | | | | | | Serum Chem |
| Afucosylated | 5 | Naïve | 1 × 10 | mg/kg | Day 0 | i.v. | CBC |
| Ab-1 | | | | | | | |
| (Low dose) | | | | | | | |
| Afucosylated | 5 | Naïve | 1 × 20 | mg/kg | Day 0 | i.v. | |
| Ab-1 | | | | | | | |
| (High dose) | | | | | | | |

TABLE 9
Study Readout Design in Imiquimod Induced Skin Inflammation Model in Cyno.

| Tissue | Sample | Time Points | Activity | Data |
|---|---|---|---|---|
| Blood | RBS lysed | Day 0 (pre dose), | Flow cytometry | CD161+ cell depletion |
| | blood | Day 1, 4, 7, 10, 14 | | |
| Skin | Biopsy | Day 7, Day 15 | Flow cytometry | CD161+ cell presence and cytokine status |
| | | | | CD161+ cell depletion |
| | | Day 7, Day 15 | MSD | Inflammatory Cytokines in skin (w/o and w/ treatment) |
| | | Day 0, Day 7, | Bulk Seq | Transcriptomic changes due to depletion of CD161+ cells |
| | | Day 15 | | Biomarkers of response |
| | | Day 0, Day 7, | Histology | Semi quantitative analysis (mild, moderate, severe) through |
| | | Day 15 | (H&E) | HE-stained slices of skin for assessment of skin damage and |
| | | | | inflammatory infiltration |
| | | Day 0, Day 7, | IHC | Influx of T cells in the skin during inflammation |
| | | Day 15 | | Decreased number of cells post anti-CD161 Ab treatment |

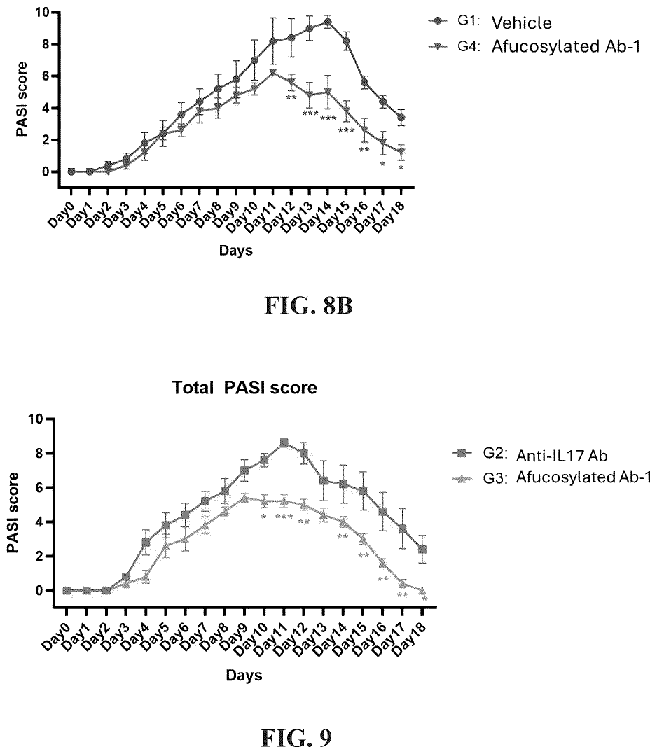

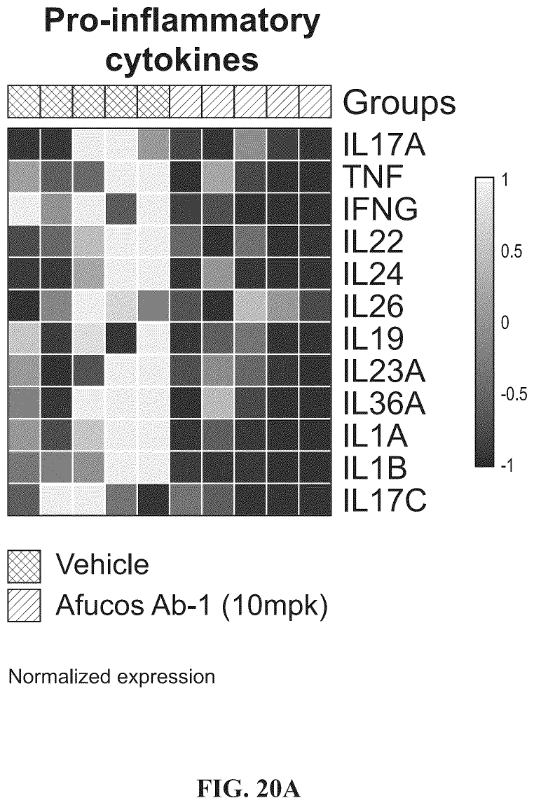

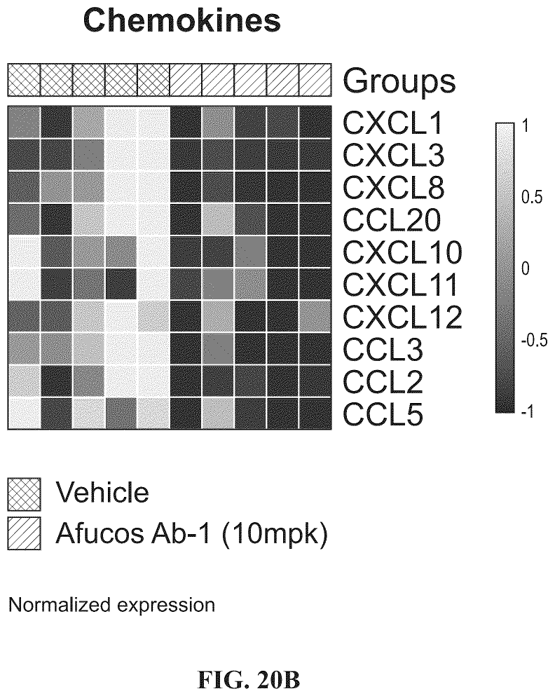

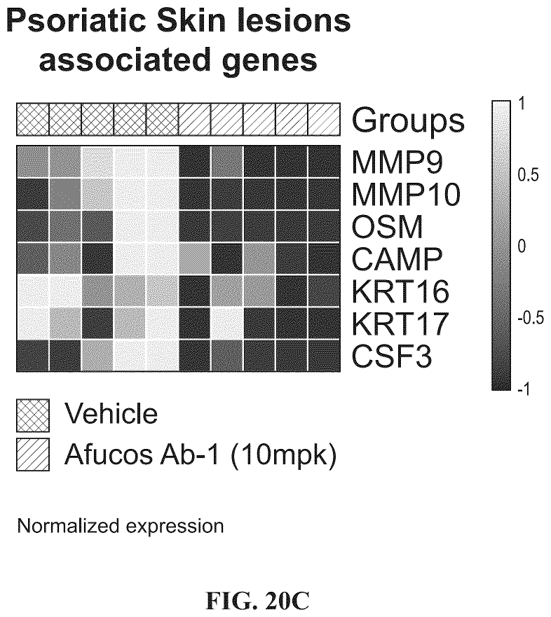

As shown in FIGS. 8A-8B, afucosylated Ab-1 significantly reduced skin inflammation in the IMQ mediated Psoriasis model at both 10 mg/kg (FIG. 8A) and 20 mg/kg (FIG. 8B) evaluated based on standard PASI (Psoriasis Area and Severity index) score.

The ability of afucosylated Ab-1 to reduce skin inflammation compared to a reference anti-IL17 antibody was also assessed. As shown in FIG. 9, afucosylated Ab-1 significantly reduced skin inflammation in the IMQ mediated Psoriasis model relative to the reference anti-IL17 antibody evaluated based on standard PASI (Psoriasis Area and Severity index) score.

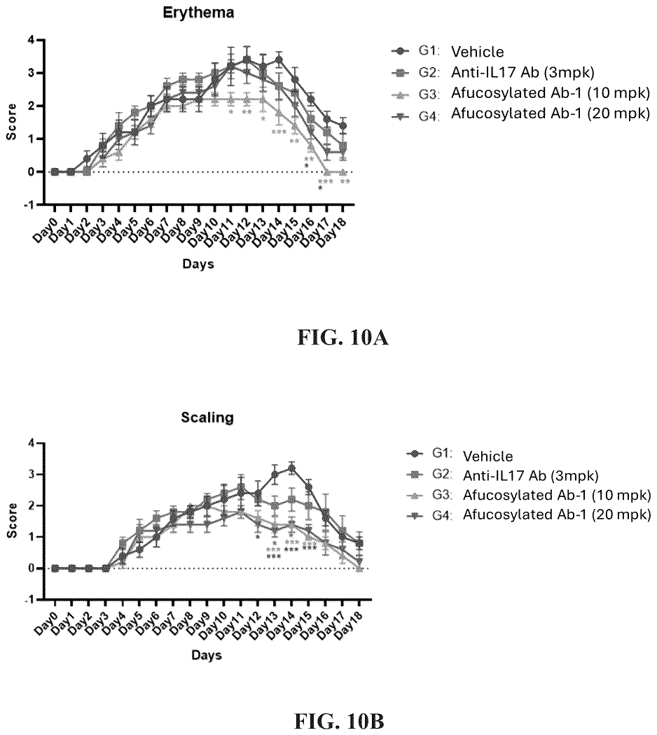

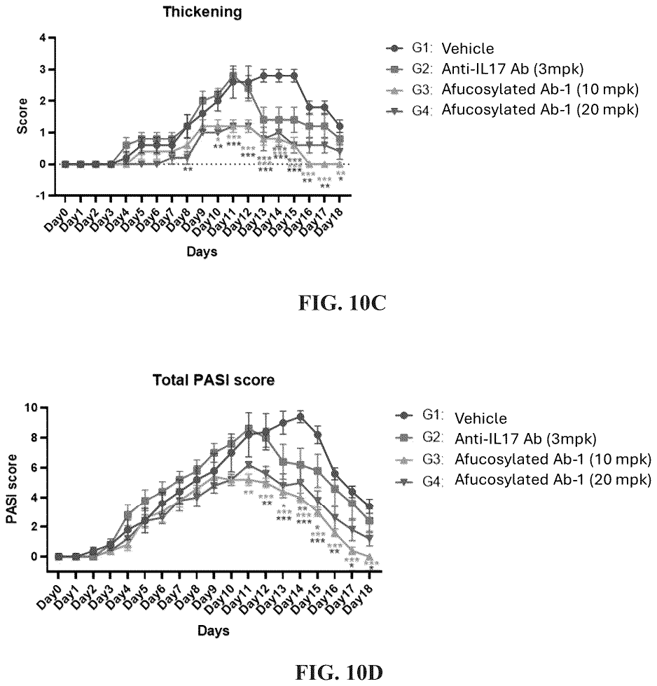

As shown in FIGS. 10A-10D, each component of the PASI score (erythema, scaling, thickening) was reduced in the afucosylated Ab-1 treatment group-FIG. 10A-erythema, FIG. 10B-scaling, FIG. 10C-thickening, and FIG. 10D-total PASI score.

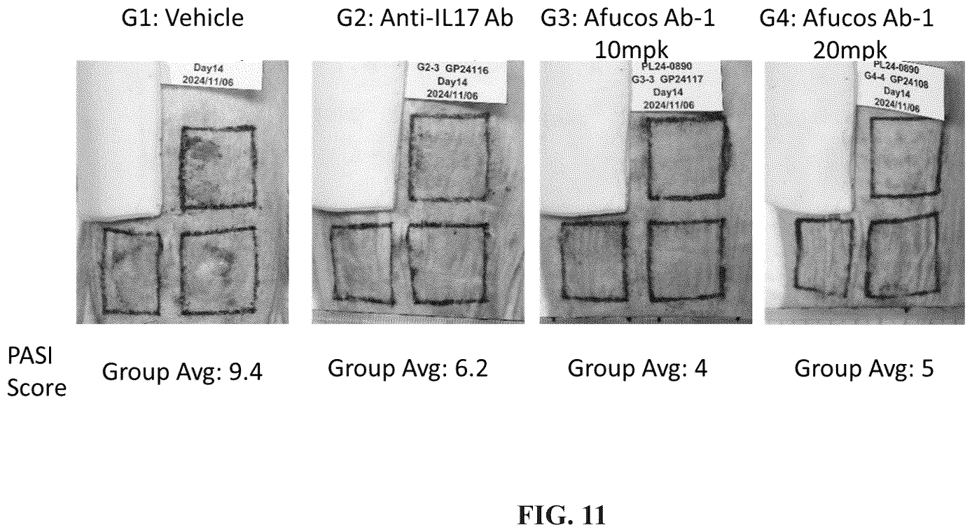

As shown in FIG. 11, skin inflammation was further visibly reduced in the afucosylated Ab-1 antibody treatment group.

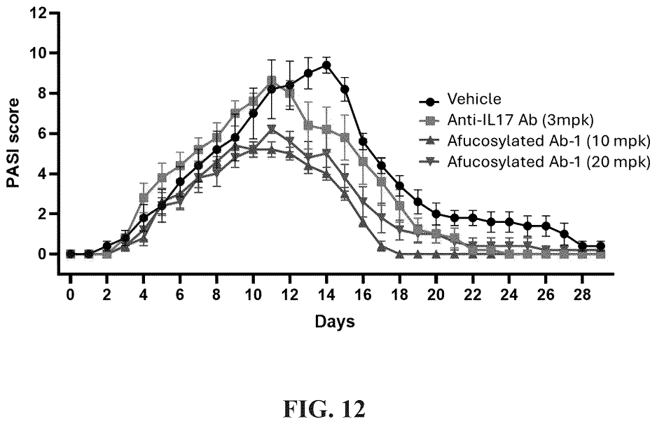

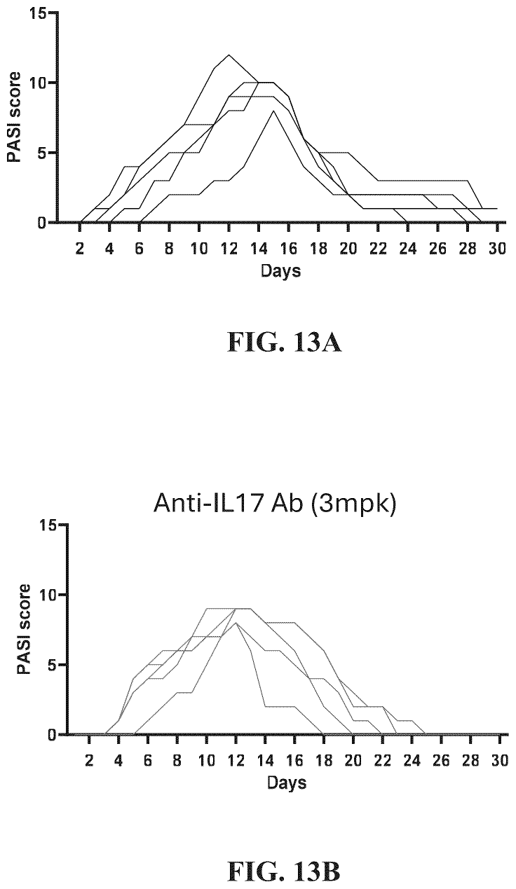

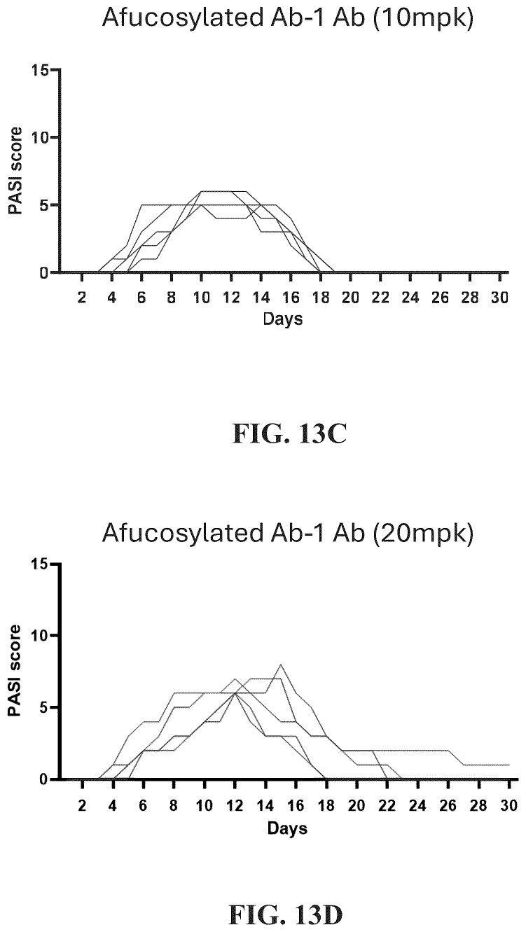

FIG. 12 further shows that the total PASI score (erythema, scaling, thickening) was reduced in the afucosylated Ab-1 antibody treatment group through day 29, showing afucosylated Ab-1 significantly reduced skin inflammation and drove faster disease resolution. FIGS. 13A-13D show the total PASI score for each individual non-human primate showing reduction in the total PASI score in the afucosylated Ab-1 treatment group (10 mg/kg (FIG. 13C) and 20 mg/kg (FIG. 13D)) relative to the vehicle control (FIG. 13A) and the reference anti-IL-17 antibody (3 mg/kg) (FIG. 13B) through day 29.

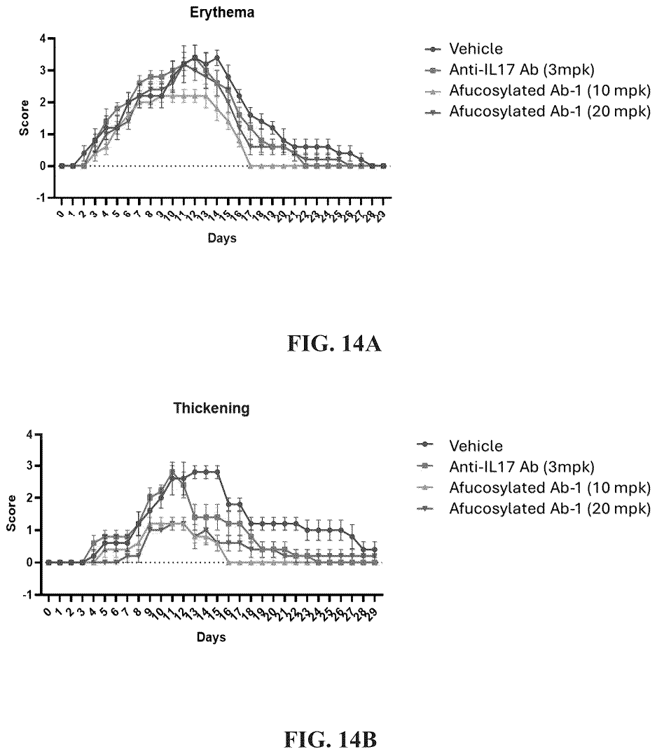

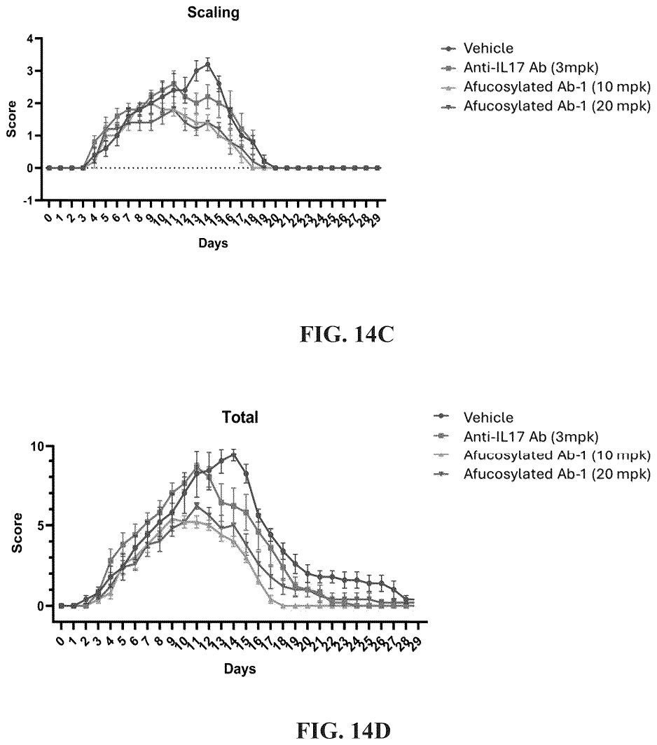

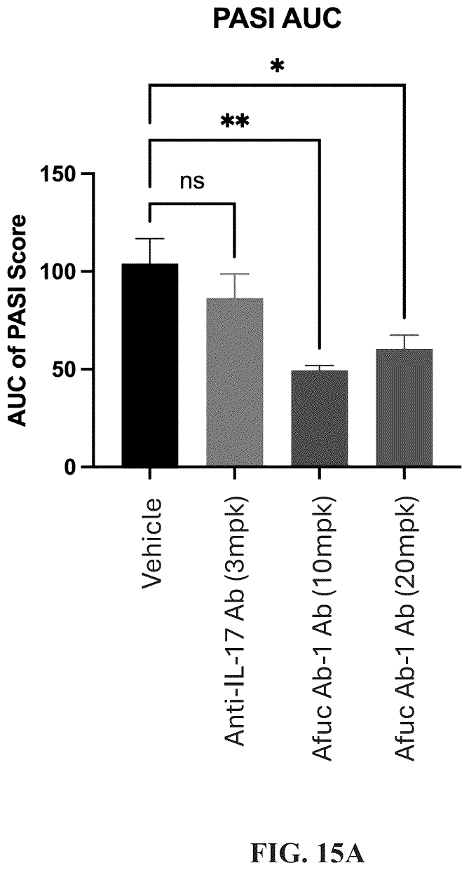

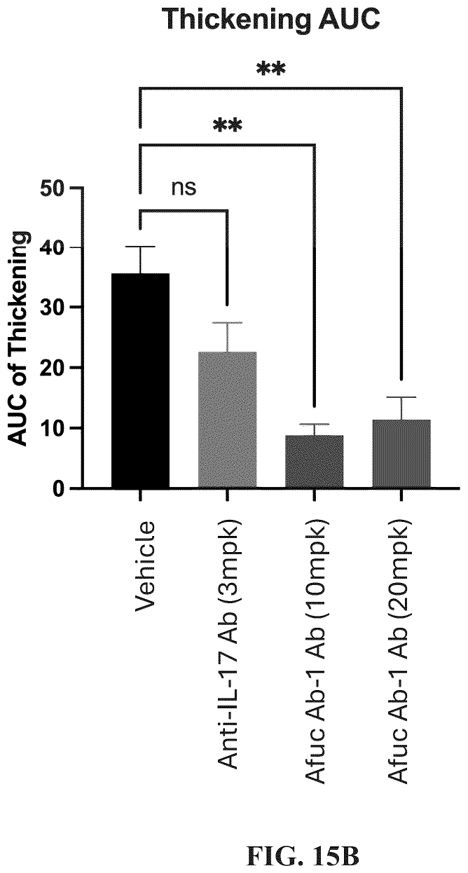

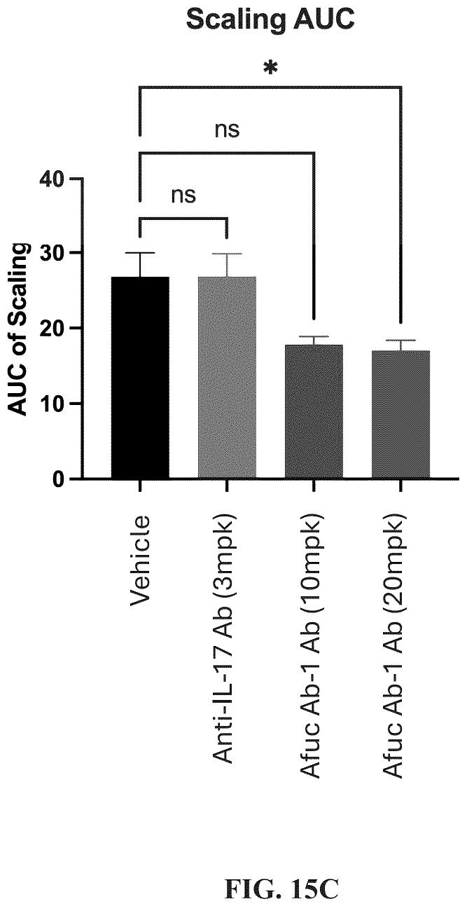

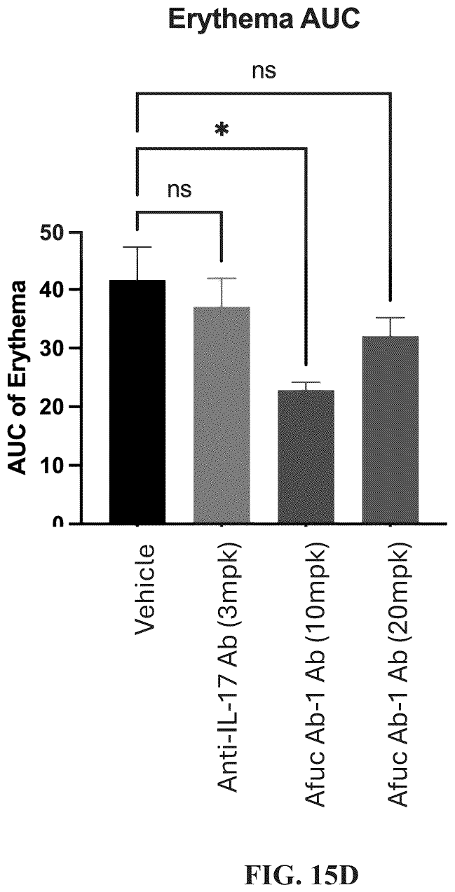

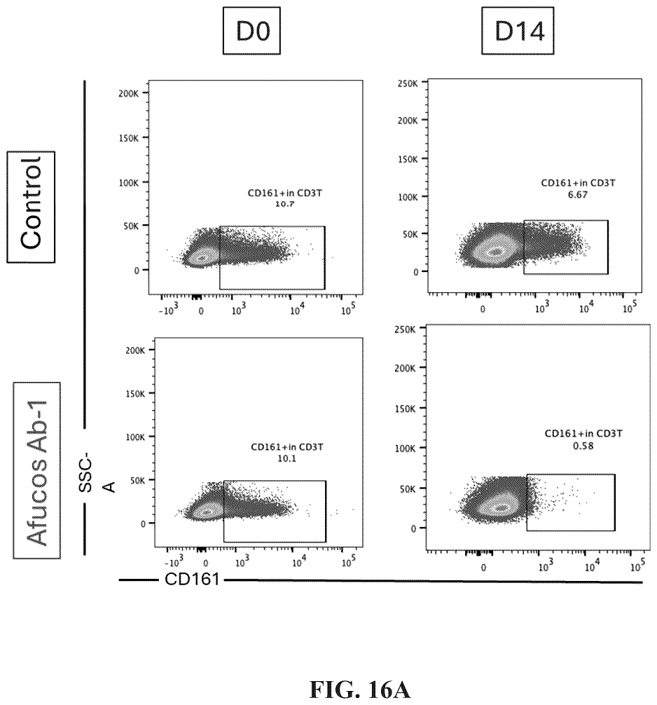

FIGS. 14A-14D show reduction in each component of the total PASI score (erythema (FIG. 14A), scaling (FIG. 14B), thickening (FIG. 14C)) and the total PASI score (FIG. 14D) in the afucosylated Ab-1 treatment group (10 mg/kg or 20 mg/kg) relative to the vehicle control and the reference anti-IL-17 antibody (3 mg/kg) through day 29. FIGS. 15A-15D further show a reduction in each component of the total PASI score (erythema (FIG. 15D), scaling (FIG. 15C), thickening (FIG. 15B)) and the total PASI score (FIG. 15A) in the afucosylated Ab-1 treatment group (10 mg/kg or 20 mg/kg) relative to the vehicle control and the reference anti-IL-17 antibody (3 mg/kg) through day 29.

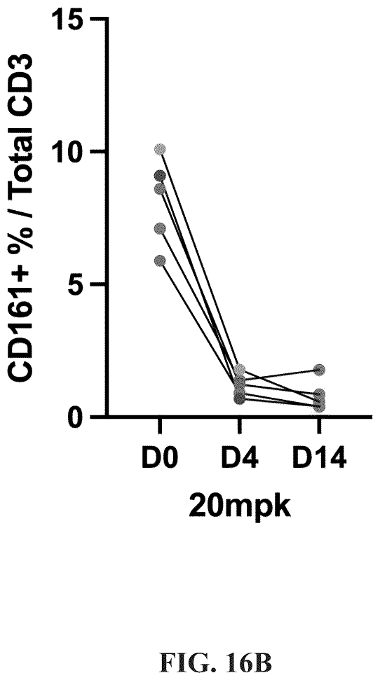

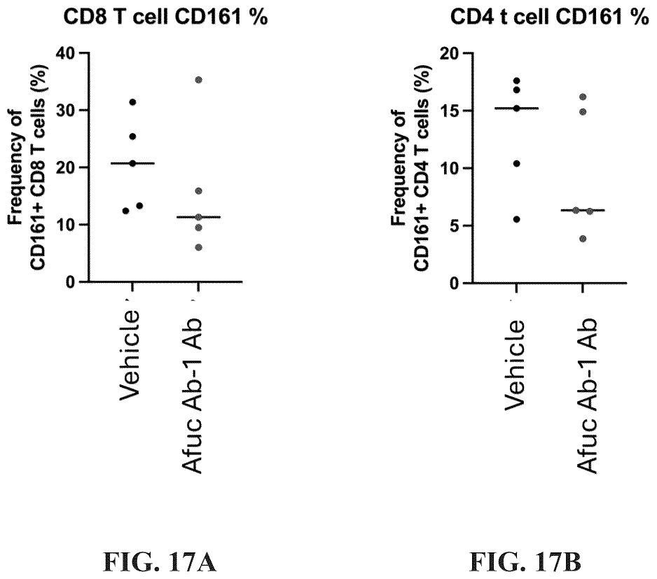

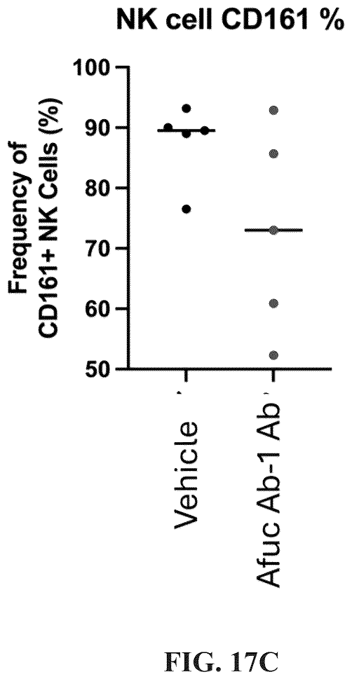

FIGS. 16A-16B shows the depletion of CD161 expressing T cells from the peripheral blood of the non-human primates treated with anti-CD161 antibody afucosylated Ab-1. FIGS. 17A-17C further show the reduction of CD161 expressing CD8+ T cells (FIG. 17A), CD4+ T cells (FIG. 17B), and NK cells (FIG. 17C) in intestinal biopsies after treatment with afucosylated Ab-1.

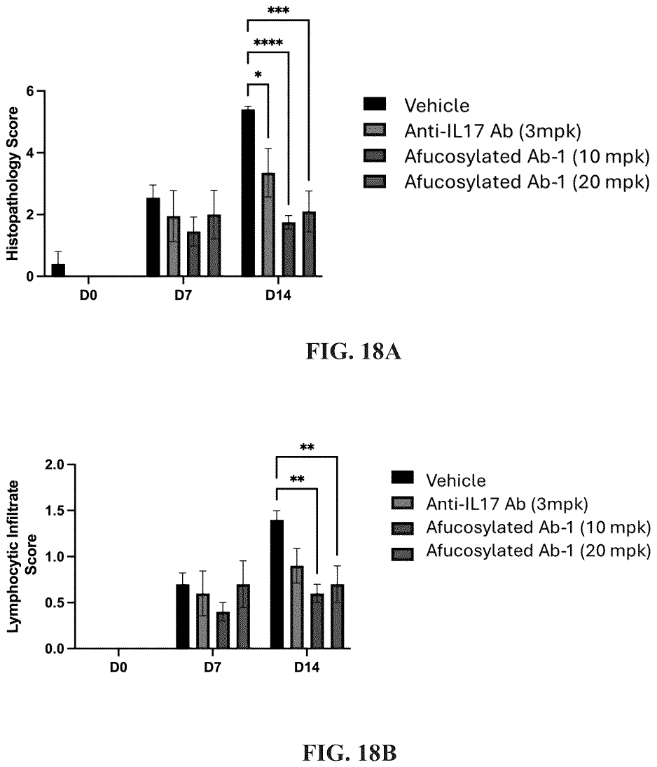

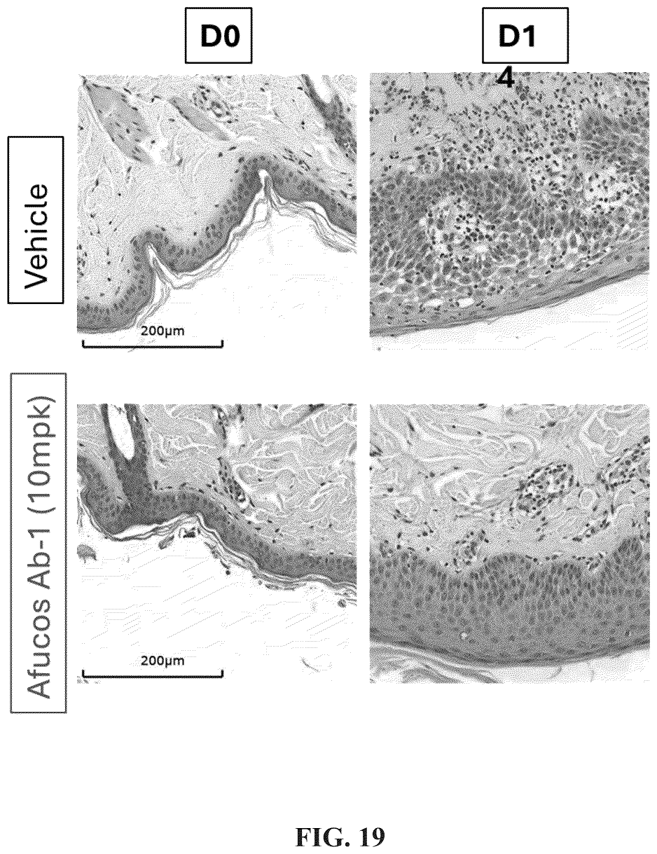

FIGS. 18A-18B and FIGS. 19A-19B shows a significant reduction in skin inflammation in the IMQ psoriasis model as assessed by histopathology score (FIG. 18A) and a lymphocytic infiltrate score (FIG. 18B). The score were determined utilizing the Baker Scoring System with the scores set forth below in Table 10.

TABLE 10
Reduction in Skin Inflammation in IMQ Psoriasis
Model Utilizing Baker Scoring System.

| Layers | Feature | Score |
|---|---|---|
| Keratin | Munro Abscess | 2.0 |
| | Hyperkeratosis | 0.5 |
| | Parakeratosis | 1.0 |
| Epidermis | Thinning over papillae | 0.5 |
| | Rete ridges appearance | 1.5 |
| | Acanthosis | 0.5 |
| | Lack of granular layer | ND |
| Dermis | Lymphocytic infiltrate | ND |
| | Mild | 0.5 |
| | Moderate | 1.0 |
| | Severe | 2.0 |

FIGS. 20A-20C further show the reduction in key mediators of skin inflammation, including pro-inflammatory cytokines (FIG. 20A), chemokines (FIG. 20B), and psoriatic skin lesion associated genes (FIG. 20C) caused by the depletion of CD161 expressing cells by the afucosylated Ab-1 antibody treatment. The gene expression data was obtained utilizing RNA sequencing of skin biopsies from the non-human primates at day 15 post treatment.

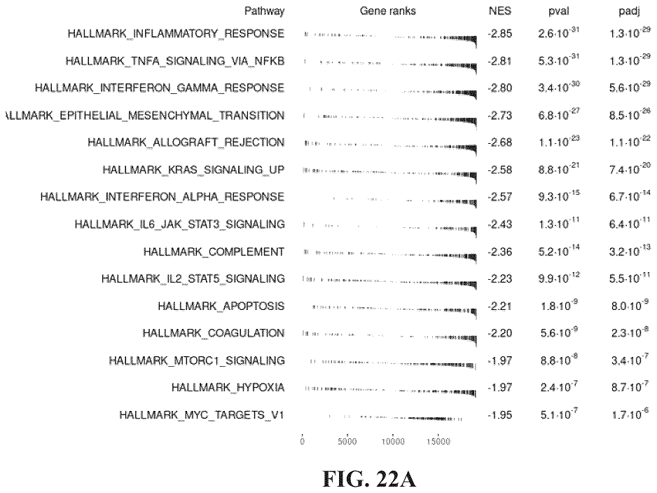

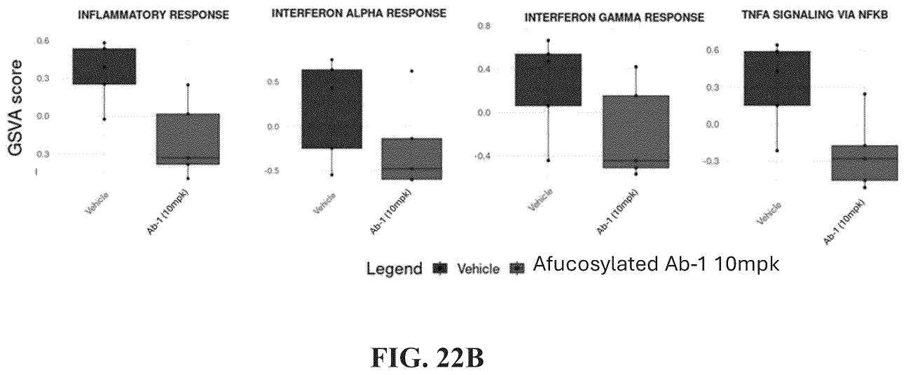

Gene Set Enrichment Analysis (GSEA) of bulk RNA-sequencing data from Day 15 skin biopsies in the NHP imiquimod (IMQ) model comparing Ab-1 (10 mg/kg)-treated animals to vehicle controls further shows significant downregulation of multiple inflammatory pathways following afucosylated Ab-1 treatment (adjusted p<0.05) (FIG. 22A). Likewise, Gene Set Variation Analysis (GSVA) quantifying the pathway-specific enrichment scores for each individual sample shows marked downregulation of the top modulated pathways including key inflammatory signals, including interferon pathways, in response to afucosylated Ab-1 treatment (FIG. 22B). The pathways include, inflammatory response, interferon alpha response, interferon gamma response, and TNFA signaling via NFKB.

### 6.4 Example 4. In Vivo Assessment of Anti-CD161 Antibody Afucosylated Ab-1 in the Treatment of Skin Inflammation

The ability of afucosylated Ab-1 to treat skin inflammation in vivo was assessed.

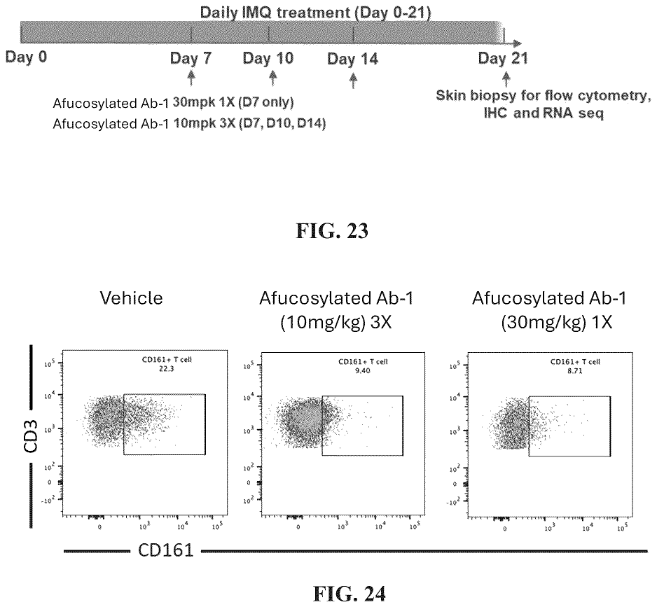

Briefly, the imiquimod induced skin inflammation model in cynomolgus macaque was utilized according to the general study design set forth in FIG. 23, wherein IMQ was applied for 21 days, the time point when cell migration to the skin was established, afucosylated Ab-1 (and controls) administered when the cells were found to be present in the skin, and the PASI score and skin depletion evaluated post afucosylated Ab-1 administration. The calculation of CD161+ T cells in the skin was performed by comparing numbers in treated animals vs. average of control group (day 21).

### 6.4.1 Afucosylated Ab-1 Mediated Target Depletion of CD161+ T Cells in Inflamed Skin.

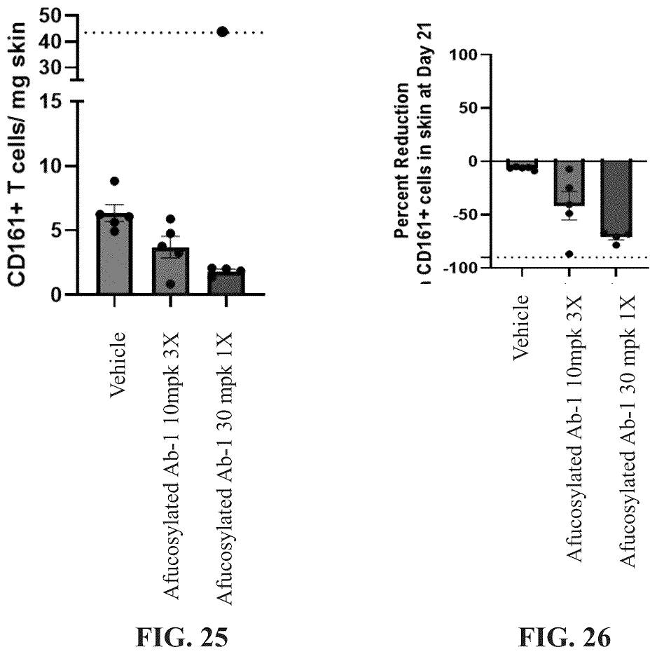

Skin biopsies taken at day 21 showed a significant reduction in CD161+ T cells at the inflamed site (FIG. 24, FIG. 25, and FIG. 26) in the afucosylated Ab-1 treatment groups (3× dose of 10 mg/kg treatment group; and single dose of 30 mg/kg treatment group).

### 6.4.2 Afucosylated Ab-1 Mediated Decrease in PASI Score.

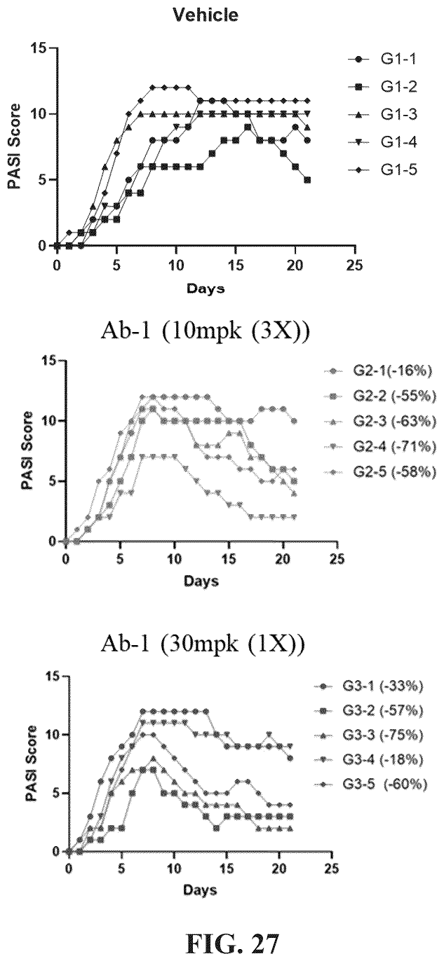

As shown in FIG. 27, afucosylated Ab-1 treatment mediated a substantial decrease in PASI scores, with a 53% reduction in the PASI score at day 21 in the 3× dose of 10 mg/kg treatment group and a 49% reduction in the PASI score at day 21 in the single dose of 30 mg/kg treatment group. Both afucosylated Ab-1 dosing regimens (3× dose of 10 mg/kg treatment group; and single dose of 30 mg/kg treatment group) elicited a strong initial treatment response and were well tolerated.

### 6.4.3 Afucosylated Ab-1 Significantly Reduces Multiple Inflammatory Pathways.

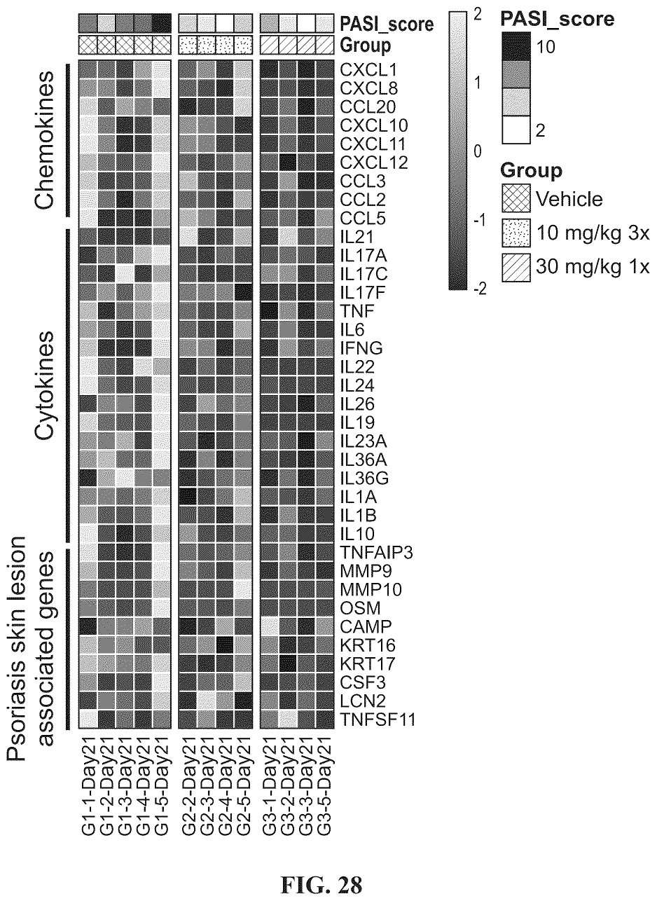

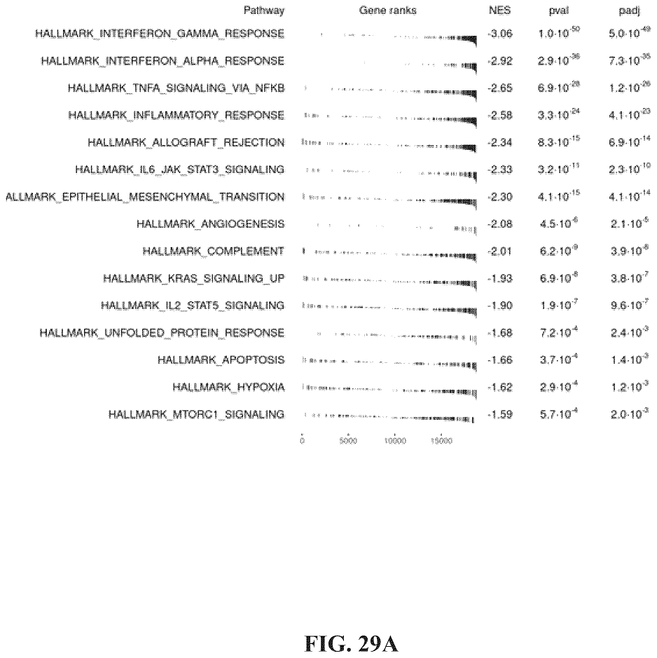

As shown in FIG. 28, RNA seq analysis of skin biopsies at day 21 showed that Ab-1 mediates a significant reduction in inflammatory mediators, including proinflammatory chemokines, proinflammatory cytokines, and psoriasis skin lesion associated genes.

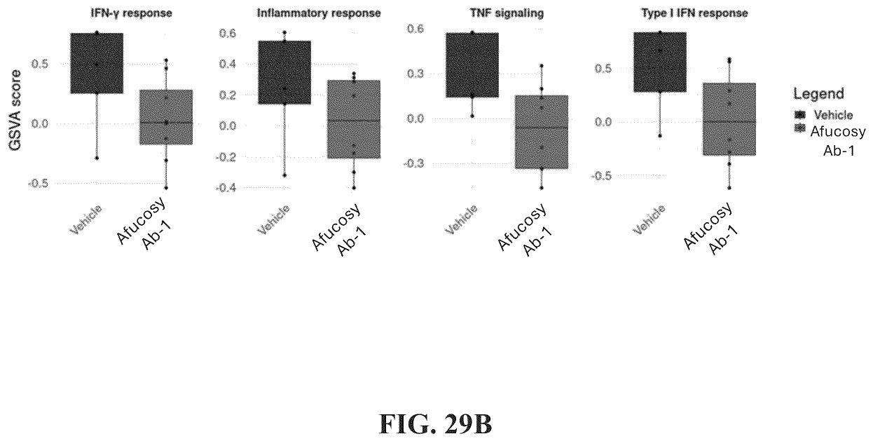

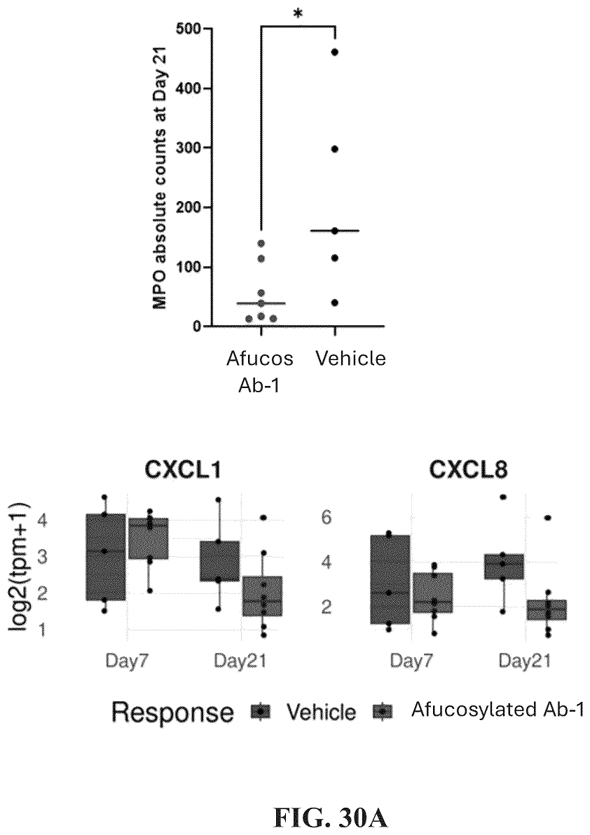

Gene Set Enrichment Analysis (GSEA) of bulk RNA-sequencing data from Day 21 skin biopsies further shows significant downregulation of multiple inflammatory pathways following afucosylated Ab-1 treatment (adjusted p<0.05) (FIG. 29A). Likewise, Gene Set Variation Analysis (GSVA) quantifying the pathway-specific enrichment scores for each individual sample shows marked downregulation of the top modulated pathways including key inflammatory signals, including interferon pathways, in response to afucosylated Ab-1 treatment (FIG. 29B). The pathways include, inflammatory response, type 1 IFN response, interferon gamma response, and TNF.

### 6.4.4 Afucosylated Ab-1 Treatment Lowers Skin Neutrophils and Macrophages by Reducing Key Cytokine Production.

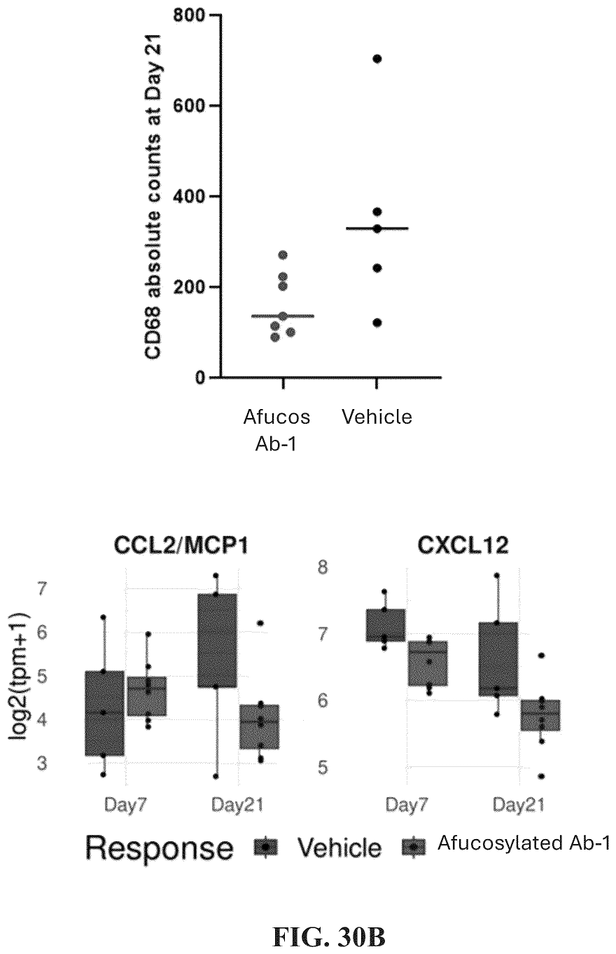

As shown in FIG. 30A and FIG. 30B, afucosylated Ab-1 further lowers the level of neutrophils (FIG. 30A) and macrophages (FIG. 30B) in the skin by reducing key cytokine production (neutrophils—CXCL1 and CXCL8; and macrophages-CCL2/MCP1 and CXCL12).

6.4.5 Afucosylated Ab-1 Treatment Reduced IFN Pathways More Effectively than Anti-IL23 and Anti-TNF Antibodies.

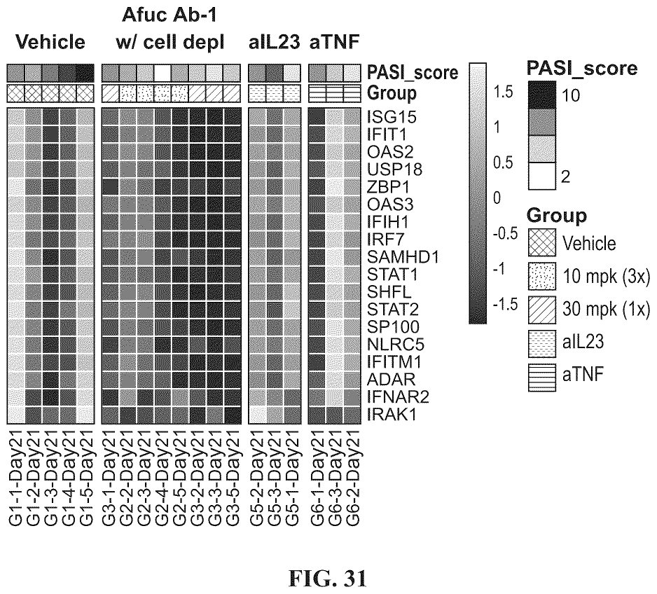

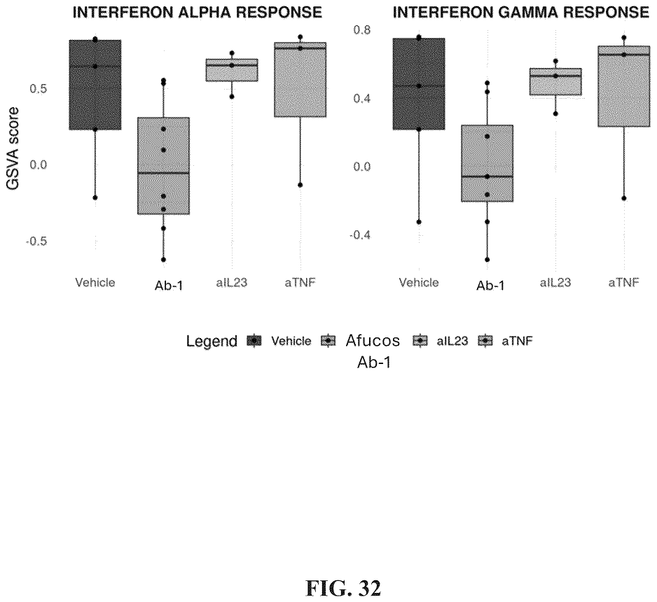

GSEA analyses of skin biopsies revealed Interferon-a and Interferon-g response pathways to be the most differentially expressed between afucosylated Ab-1 treatment and both anti-IL23 antibody and anti-TNF antibody treatments (FIG. 31). Comparison of IFN pathway gene expression by GSVA scoring show decreased levels in afucosylated Ab-1 treated animals vs both anti-IL-23 antibody and anti-TNF antibody treated animals (FIG. 32).

### 6.4.6 Conclusions

Collectively, the above results demonstrate, inter alia, afucosylated Ab-1 mediated reduction in established inflammation, single dose efficacy of afucosylated Ab-1, afucosylated Ab-1 mediated T cell depletion in the skin, afucosylated Ab-1 mediated reduction in inflammatory cytokines and chemokines, and a more effective reduction in IFN pathways by afucosylated Ab-1 relative to both anti-IL23 antibody and anti-TNFα antibody therapy.

### 6.5 Example 5. In Vitro CD161 Cell Depletion Reduces Multiple Inflammatory Mediators in Tissue Samples from Crohn's Disease Patients

6.5.1 CD161+ CD4+ T cells Produce Inflammatory Cytokines in Tissue Samples from Crohn's Disease Patients.

The level of pro-inflammatory cytokines produced by fresh tissue samples from Crohn's disease patients was assessed in vitro.

Crohn's disease samples utilized included colon resection samples from six individual patients. These samples were treated with collagenase enzyme to generate single cell suspensions that were stained for CD161 expression on CD4 and CD8 T cells and analysed by flow cytometry. A portion of the single cell suspensions were treated with PMA and Ionomycin and Brefeldin A for 4 hours followed by surface receptor staining for CD161, CD4 and CD8 and intracellular staining for IL 17F, IL-22, IFNγ, and TNFα. These samples were then analysed by flow cytometry.

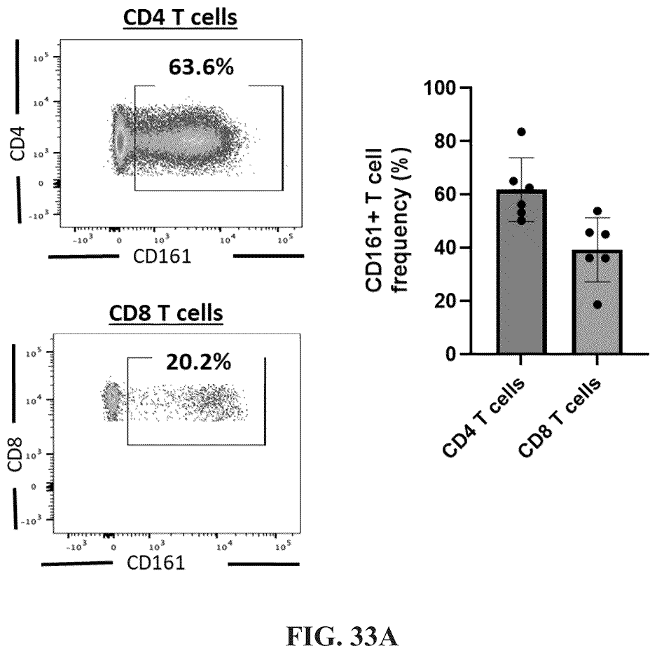

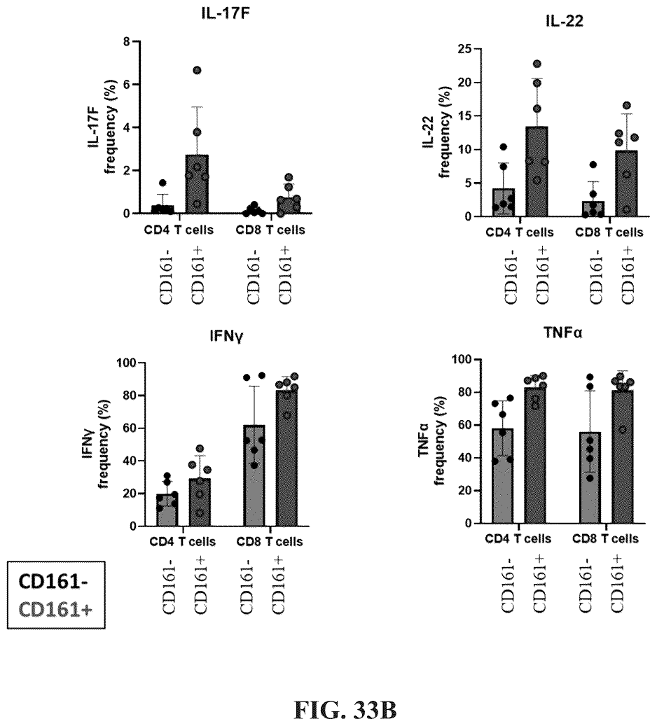

As shown in FIG. 33A high numbers of CD161+ T cells, particularly CD161+CD4+ T cells, were present in the intestinal tissue of the Crohn's disease patients. FIG. 33B further showed that CD161+ T cells in the intestinal tissue of the Crohn's disease patients produced elevated levels of proinflammatory cytokines (e.g., IL-17F, IL-22, IFNγ, and TNFα) (relative to cells that do not express CD161) showing that CD161+ cells are highly pathogenic in context of inflammatory bowel disease (FIG. 33B).

6.5.2 In vitro CD161+ Cell Depletion Reduces Multiple Inflammatory Mediators in Samples from Crohn's Disease Patients.

The effect of CD161+ cell depletion in samples from Crohn's disease patients on the level of multiple inflammatory mediators was assessed in vitro.

Crohn's disease samples utilized included colon resection samples from two individual patients. These samples were treated with collagenase enzyme to generate single cell suspensions. The samples were either subjected to CD161+ cell depletion using Miltenyi magnetic bead-based separation in vitro, or control; and stimulated using one of the following treatments: no treatment, Immunocult™ anti-CD3/CD28 activator, anti-IL23/IL-1β antibodies or anti-IL-18/IL-12 antibodies. The samples were cultured overnight. Culture supernatants were collected the following day and cytokine secretion was quantified using Meso Scale Discovery (MSD).

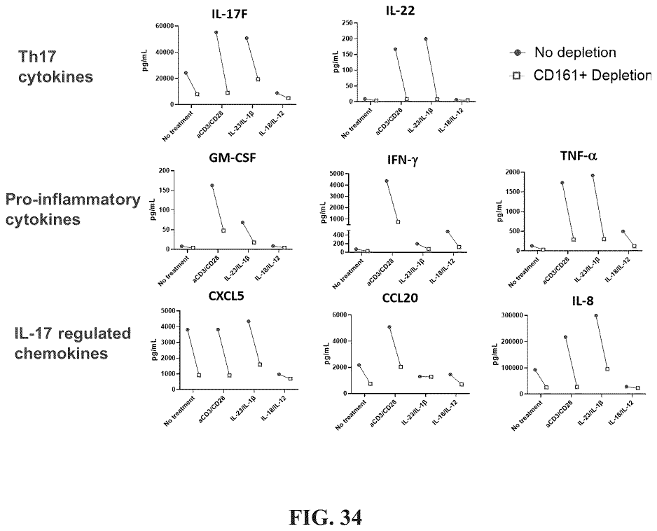

High levels of pro-inflammatory cytokines and chemokines in tissue samples from Crohn's disease patients (including Th17 cytokines-IL17f, IL-22; Pro-inflammatory cytokines-GMCSF, IFNγ, TNFα; and IL 17 regulated chemokines-CXCL5, CCL20, and IL8) demonstrated a high baseline of inflammation (FIG. 34). Multiple inflammatory molecules were further increased by treatment with cytokines alone without TCR stimulation demonstrating capability of CD161+ cells to respond to disease tissue milieu (FIG. 34).

As shown in FIG. 34, CD161 cell depletion (with magnetic beads) reduced multiple inflammatory mediators in samples from Crohn's disease patients, including, Th17 cytokines-IL17f, IL-22; Pro-inflammatory cytokines-GMCSF, IFNγ, TNFα; and IL17 regulated chemokines-CXCL5, CCL20, and IL8. The reduction of CXCL5, CCL20 and IL-8 may indicate the impact of CD161+ cell depletion on other cells in colon tissue including epithelial cells and macrophages.

### 6.6 Example 6. Comparison of Ab-1 to Anti-CD161 Antibody HP-3G10

An analysis comparing the binding affinity and epitope binding of Ab-1 to the commercially available anti-CD161 antibody HP-3G10 was conducted.

### 6.6.1 Ab-1 is a Stronger Binder to Human and Cyno CD161 Compared to HP-3G10.

First, analysis comparing the binding affinity of Ab-1 to the commercially available anti-CD161 antibody HP-3G10 to human and cyno CD161 was conducted.

Briefly, 100,000 HEK cells expressing human CD161 and HEK cells expressing Cyno CD161 were aliquoted in different wells of a cell culture plate. The indicated antibodies were titrated starting at 100 nM with 3× dilutions until 0.05 nM and incubated with the cells at 4° C. for 1 hour. After the incubation period, the cells were washed two times followed by incubation with an anti-hFc-A647 conjugated secondary antibody at a 1:1000 dilution. The cells were incubated on ice for 30 min followed by washes and analysed on flow cytometer.

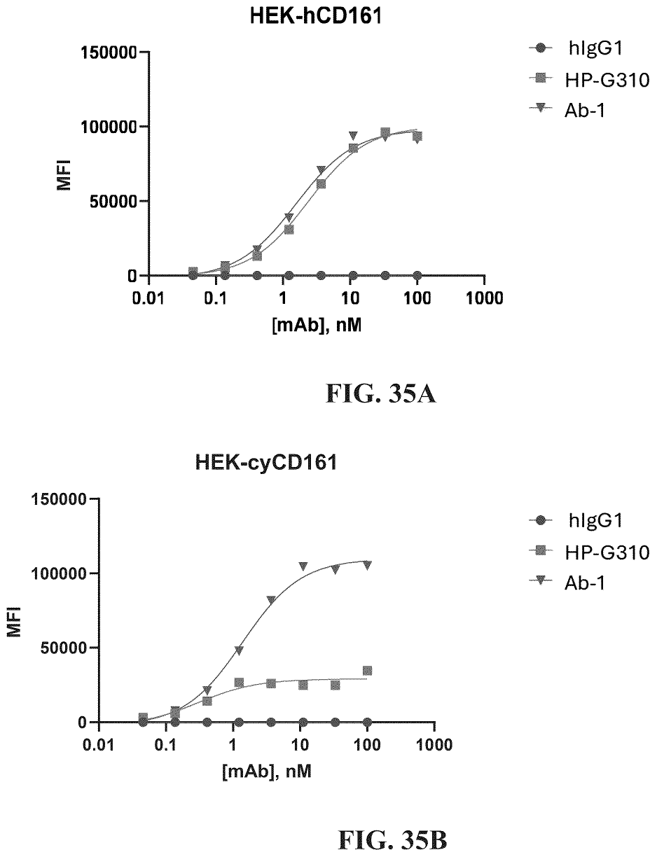

As shown in FIG. 35A and FIG. 35B, Ab-1 bound human CD161 slightly stronger than HP-3G10 (EC50 for HP3G10 is 2.38 nM and for Ab-1 is 1.56 nM). Ab-1 bound cyno CD161 significantly stronger than HP-3G10.

6.6.2 Ab-1 Does Not Compete For Human CD161 Binding with HP-3G10.

Further, the epitope binding of Ab-1 and HP-3G10 was assessed.

First, Octet based epitope binning was utilized. Briefly, a sandwich format was used. Ab-1 was first immobilized on the biosensor followed by addition of human-CD161-Fc protein. The tip was then dipped into the reference antibody HP3G10.

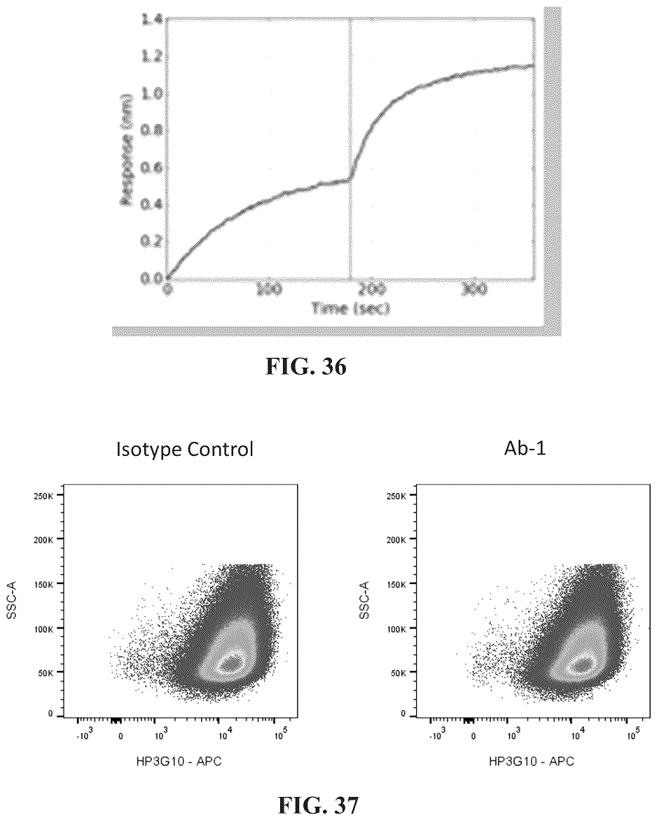

As shown in FIG. 36, a positive signal or shift in wavelength was observed from binding of HP3G10. This indicates that HP3G10 is not competing with Ab-1 for the same binding site, and therefore belong to different epitope bins. Overall, Ab-1 does not compete for human CD161 binding with HP-3G10.

Second, a flow cytometry based analysis was conducted. Briefly, Jurkat cells overexpressing CD161 were incubated with 100 nM of either unconjugated isotype control or Ab-1 at 4° C. for 30 min. The cells were then washed twice and stained with APC conjugated anti-CD161 antibody (clone HP3G10) at 4° C. for 30 min followed by washes and analyses on the flow cytometer. The APC conjugated HP3G10 antibody used here was bought from Biolegend Cat #339912.

As shown in FIG. 37, Ab-1 does not compete with HP-3G10 for binding to CD161 as there is no loss of fluorescence signal relative to the isotype.

### 6.7 Example 7. Afucosylated Ab-1 Induction of Antibody Dependent Cellular Phagocytosis (ADCP)

The ability of afucosylated Ab-1 to induce ADCP mediated killing of CD161 expressing cells was assessed by standard methods known in the art and summarized below.

Briefly, monocytes were isolated from healthy donor PBMCs using Miltenyi classical monocyte isolation kit and plated at 1e6/mL in 6-well tissue culture plates in IMDM media with 10% human serum. On Day 7 adherent macrophages were collected and resuspended in serum-free IMDM. Total T cells were also isolated from the same PBMC donor using Miltenyi Pan T cell isolation kit. T cells were labeled with Calcein AM fluorescent dye and stained with BV421 anti-human CD161 (clone HP3G10) to perform sorting into CD161 positive and negative subsets. Sorted T cells were plated at 400,000 cells/well in 100 uL serum-free IMDM. Afucosylated Ab-1 was added to the T cells at a final concentration of 2 nM. Media alone was used as a no antibody control. Macrophages were added at 50,000 cells/well for a final E: T ratio of 1:8. The co-culture was incubated for 2 hr at 37° C. Cells were stained with BV785 anti-human CD11b to distinguish macrophages and run on flow cytometer. The % phagocytosis was calculated based on CD11b+ Calcein AM+ event.

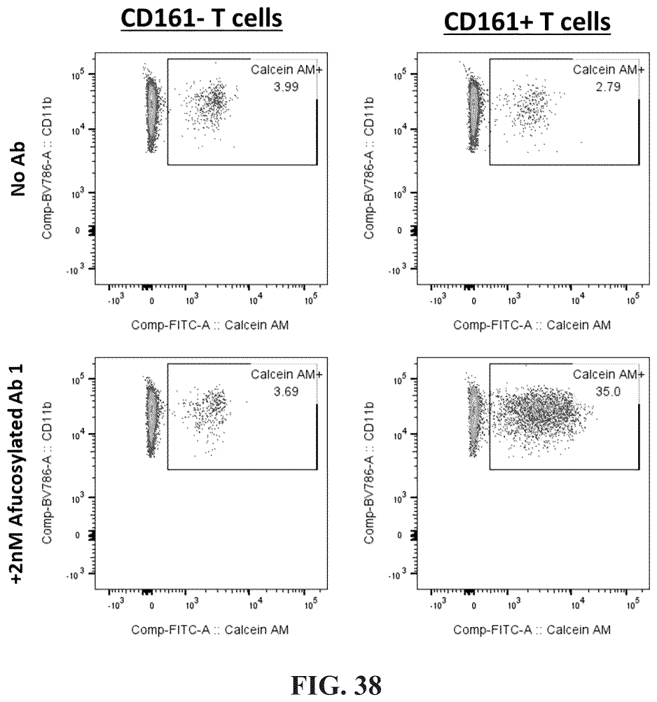

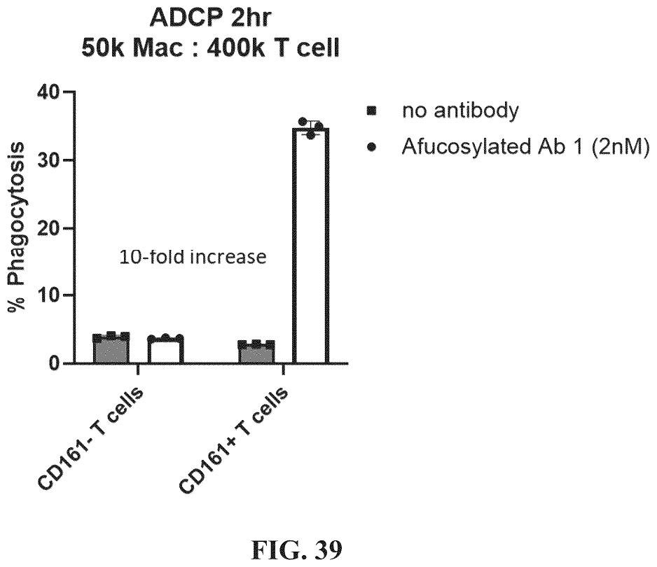

As shown in FIG. 38 and FIG. 39, afucosylated Ab-1 induced a 10 fold increase in ADCP of CD161+ cells as compared to no antibody control. Since afucosylated Ab-1 is a highly specific antibody for CD161, the CD161 negative T cells did not undergo ADCP higher than the no antibody control.

### 6.8 Example 8. Afucosylated Ab-1 Does Not Induce Cytokine Release Associated with Cytokine Release Syndrome (CRS)

Whether afucosylated Ab-1 induced cytokine release typically associated with CRS was assessed.

Briefly, the potential for afucosylated Ab-1 to simulate cytokine release in human whole blood was assessed in vitro. Proinflammatory cytokines evaluated included IL-2, IL-10, IL-1β, IL-4, IL-6, TNF-α, IFNγ, GM-CSF, IL17A, IL17F, IL12p70 and IFNa2a. Anti-CD20 rituximab (human IgG1; 100 ug/ml), Anti CD20 Obinutuzumab (afucosylated IgG1; 100 ug/ml), Immunocult™ anti CD3/anti CD28 activator (vendor recommended 1×), R848 (TLR7 activator; 10 ug/ml), and LPS (10 ug/ml) were used as positive controls along with the afucosylated isotype control (100 ug/ml) an no treatment used as negative controls. The afucosylated Ab-1 was tested at 100 ug/mL, 10 ug/mL, and 1 ug/mL concentrations. The antibodies were assessed in 2 different formats: plate-bound (solid phase) and soluble form (aqueous phase). Whole blood from 10 individual healthy human donors were incubated in the presence of afucosylated Ab-1 and corresponding control molecules for 24 hours. The culture supernatants were collected and tested for cytokine release by Meso Scale Discovery (MSD). All treatments were performed in triplicates per donor. Every data point is expressed as the average of triplicates for each donor.

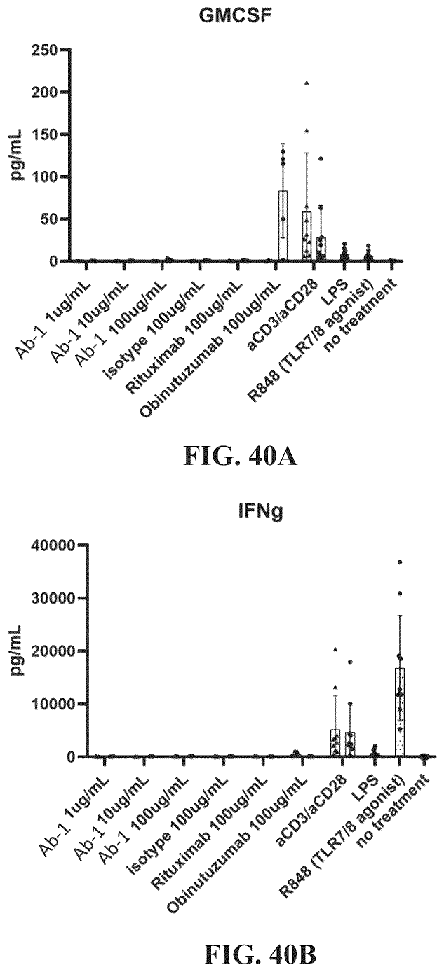

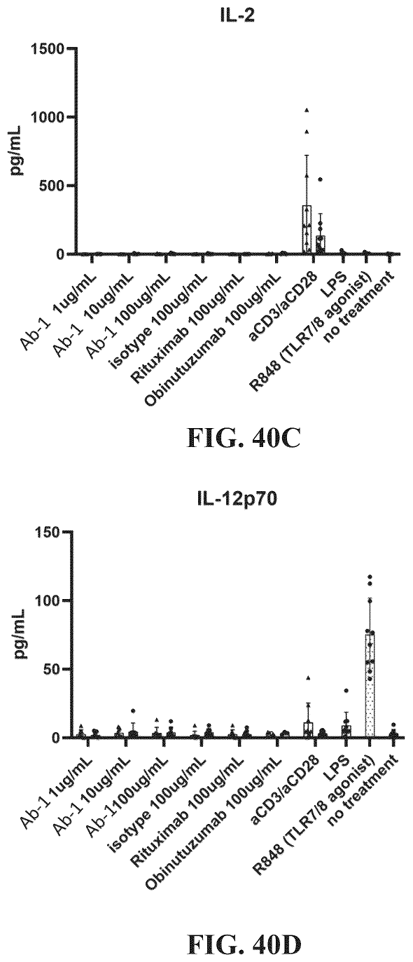

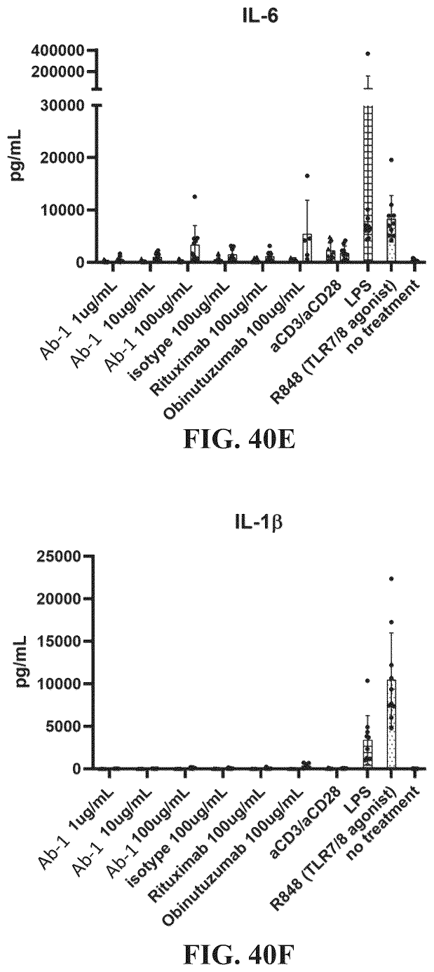

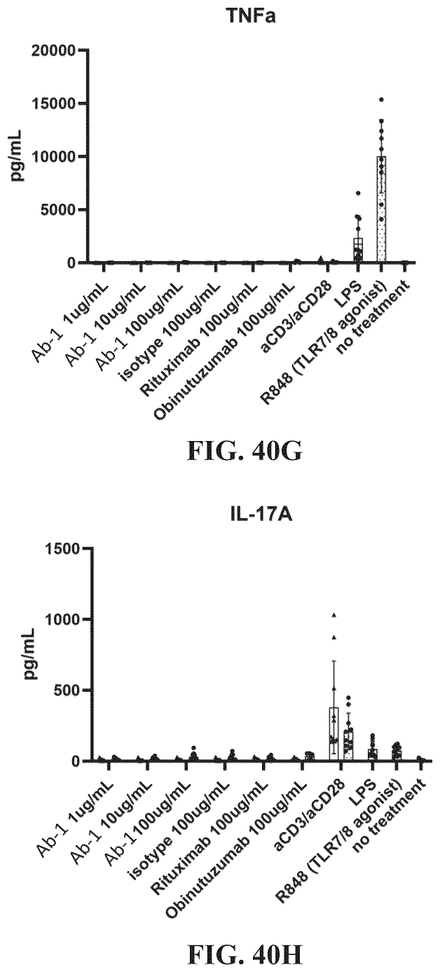

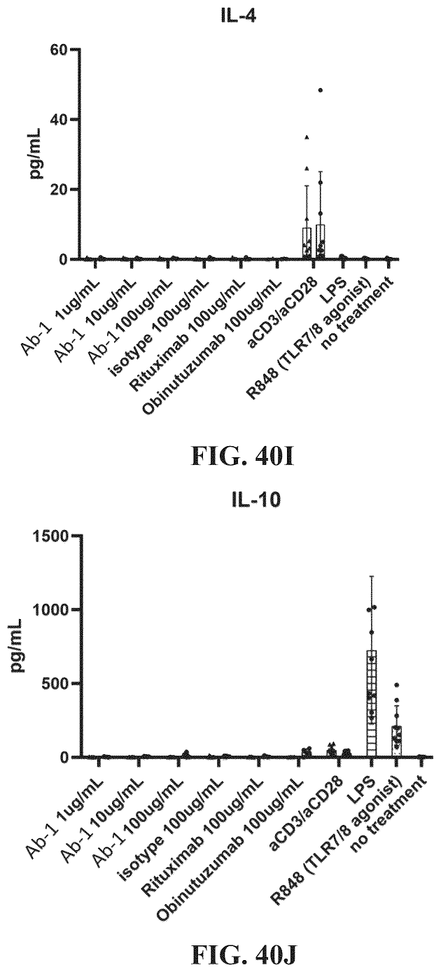

As shown in FIGS. 40A-40J, no significant increase in cytokine production was observed when whole blood was incubated with afucosylated Ab-1 in both solid (triangles) and aqueous (circles) phase formats when compared to positive controls that significantly stimulated release of the cytokines tested. A single data point in the IL-6 plot (FIG. 40E; black arrows) showed higher cytokine production in the presence of highest concentration of afucosylated Ab-1 tested. This was attributed to a single donor that showed a similar response to Obinutuzumab (afucosylated IgG1) which is an approved therapy for chronic lymphocytic leukemia, follicular lymphoma and lupus nephritis. IL17F and IFNa2a were below detection limit for the samples and not shown. These data suggest that there is a low risk of cytokine release in patients treated with afucosylated Ab-1.

### 6.9 Example 9. CD161 Cell Depletion Mediates Enhanced Reduction in Multiple Inflammatory Mediators in Colon Tissue Samples from Crohn's Disease Patients Compared to Other Therapeutics

The effect of CD161+ cell depletion in samples from Crohn's disease patients on the level of multiple inflammatory mediators compared to the use of other cytokine inhibitor treatments was assessed in vitro.

Briefly, Crohn's disease patient-derived colonic biopsies were enzymatically digested using collagenase type IV and DNase I to obtain single-cell suspensions. Dissociated cells were then subjected to magnetic separation for CD161+ cell depletion using biotinylated Ab-1 and anti-biotin MicroBeads (7 samples). The CD161− and total cell fractions were seeded in 96-well plates and activated with anti-CD3/CD28 activator and IL-2 for 24 hours. The total cell fractions were separately activated with anti-CD3/CD28 activator, IL-2 and treated with cytokine neutralizing antibodies (anti-IL-17A (ixekizumab) (7 samples), anti-TNF-α (adalimumab) (6 samples), anti-IL-17A/F (bimekizumab-bkzx) (6 samples), or anti-IgG1 control (4 samples) at a concentration of 10 ug/ml) for 24 hours. The culture supernatants were collected at 24 hours post co-culture and tested for different pro-inflammatory cytokines by Meso Scale Discovery (MSD). The fold change of cytokine produced in each treatment condition was calculated over cytokines produced in untreated samples. Anti-IL-17 neutralization blocks receptor engagement but does not prevent cytokine secretion or detection by capture antibodies. CD161; cell depletion eliminates IL-17-producing populations, resulting in a true reduction of measurable IL-17 levels in vitro.

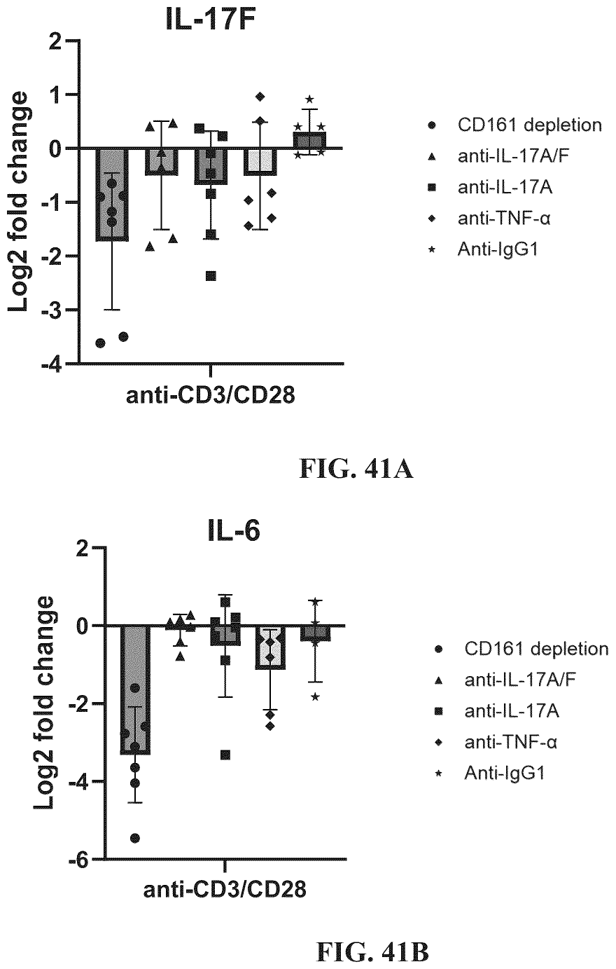

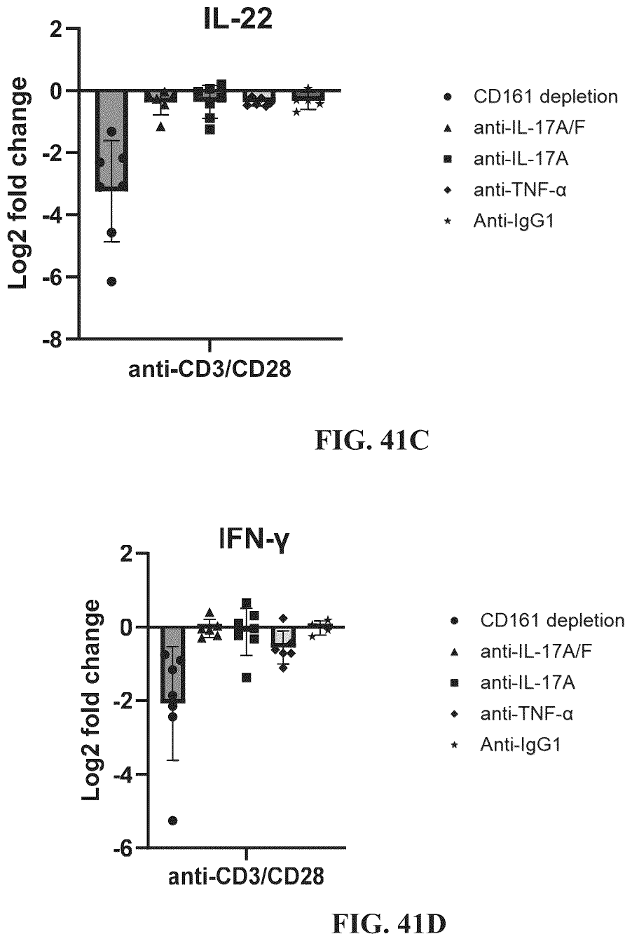

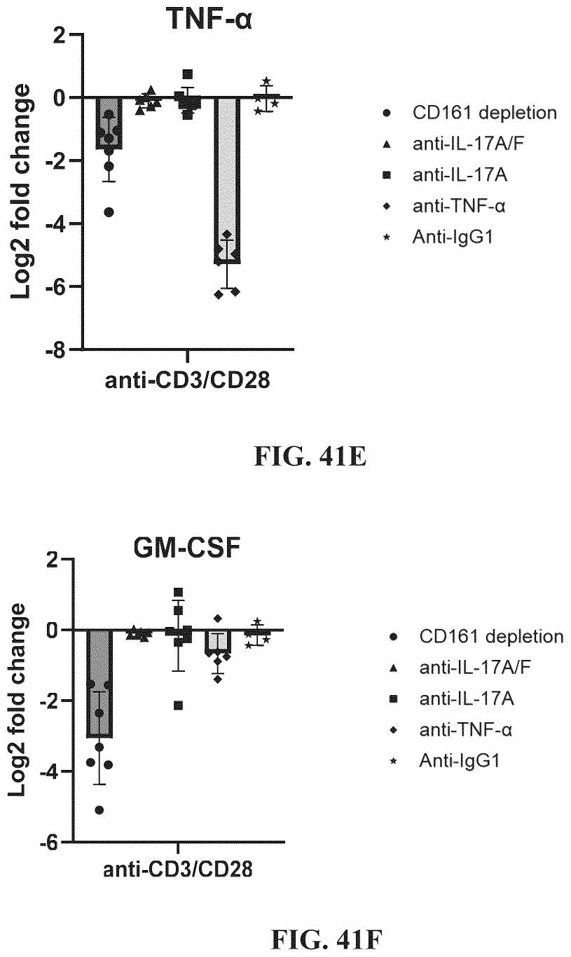

Overall, by selectively removing CD161+ cells, one was able to compare the benefit of CD161 depletion along with several neutralization antibodies to evaluate cytokine production. As shown in FIGS. 41A-41F, the fold reduction in proinflammatory cytokines was much higher when CD161+ cells were depleted than other interventions tested. Thus, depleting pathogenic CD161 cells achieves greater reduction of inflammatory cytokines than cytokine blocking therapies.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A CD161 binding protein comprising a binding domain that specifically binds CD161, wherein the binding domain comprises:
(a) a VH region comprising (i) a CDR-H1 comprising the amino acid sequence set forth in SEQ ID NO: 3 or the amino acid sequence set forth in SEQ ID NO: 3 comprising 1, 2, or 3 amino acid modifications; (ii) a CDR-H2 comprising the amino acid sequence set forth in SEQ ID NO: 4 or the amino acid sequence set forth in SEQ ID NO: 4 comprising 1, 2, or 3 amino acid modifications; and (iii) a CDR-H3 comprising the amino acid sequence set forth in SEQ ID NO: 5 or the amino acid sequence set forth in SEQ ID NO: 5 comprising 1, 2, or 3 amino acid modifications; and
(b) a VL region comprising (i) a CDR-L1 comprising the amino acid sequence set forth in SEQ ID NO: 6 or the amino acid sequence set forth in SEQ ID NO: 6 comprising 1, 2, or 3 amino acid modifications; (ii) a CDR-L2 comprising the amino acid sequence set forth in SEQ ID NO: 7 or the amino acid sequence set forth in SEQ ID NO: 7 comprising 1, 2, or 3 amino acid modifications; and (iii) a CDR-L3 comprising the amino acid sequence set forth in SEQ ID NO: 8 or the amino acid sequence set forth in SEQ ID NO: 8 comprising 1, 2, or 3 amino acid modifications.

2. A CD161 binding protein comprising a binding domain that specifically binds CD161, wherein the binding domain comprises:
(a) a VH region comprising (i) a CDR-H1 comprising the amino acid sequence set forth in SEQ ID NO: 3; (ii) a CDR-H2 comprising the amino acid sequence set forth in SEQ ID NO: 4; and (iii) a CDR-H3 comprising the amino acid sequence set forth in SEQ ID NO: 5; and
(b) a VL region comprising (i) a CDR-L1 comprising the amino acid sequence set forth in SEQ ID NO: 6; (ii) a CDR-L2 comprising the amino acid sequence set forth in SEQ ID NO: 7; and (iii) a CDR-L3 comprising the amino acid sequence set forth in SEQ ID NO: 8.

3. A CD161 binding protein comprising a binding domain that specifically binds CD161, wherein the binding domain comprises:
(a) a VH region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and
(b) a VL region comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10.

4. The CD161 binding protein of claim 1, wherein
(a) the amino acid sequence of the VH region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 9; and
(b) the amino acid sequence of the VL region is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 10.

5.-8. (canceled)

9. The CD161 binding protein of claim 1, comprising a heavy chain (HC) and a light chain (LC), wherein the amino acid sequence of the HC is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 67; and the amino acid sequence of the LC is at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 68.

10.-41. (canceled)

42. A conjugate comprising the CD161 binding protein of claim 1 and a heterologous moiety.

43.-50. (canceled)

51. A fusion protein comprising the CD161 binding protein claim 1 and a heterologous protein.

52.-56. (canceled)

57. A nucleic acid molecule encoding the CD161 binding protein of claim 1.

58.-60. (canceled)

61. A vector comprising the nucleic acid molecule of claim 57.

62.-63. (canceled)

64. A carrier comprising the CD161 binding protein of claim 1.

65. (canceled)

66. A cell or population of cells comprising the CD161 binding protein of claim 1.

67. A pharmaceutical composition comprising the CD161 binding protein of claim 1, and a pharmaceutically acceptable excipient.

68. A kit comprising the CD161 binding protein of claim 1; and optionally instructions for using the CD161 binding protein.

69. A method of manufacturing a CD161 binding protein, the method comprising:
introducing into a cell the nucleic acid molecule of claim 57;
culturing the cell under conditions that allow for expression of the CD161 binding protein; and
optionally recovering the expressed the CD161 binding protein; and
optionally purifying the expressed the CD161 binding protein from the culture.

70. A method of manufacturing an afucosylated version of a CD161 binding protein, the method comprising:
introducing into a cell that comprises a functional disruption in the fucosyltransferase 8 (FUT8) gene the nucleic acid molecule of claim 57;
culturing the cell under conditions that allow for expression of the afucosylated CD161 binding protein; and
optionally recovering the expressed the CD161 binding protein from the culture; and
optionally purifying the expressed the CD161 binding protein from the culture.

71. A method of delivering a CD161 binding protein to a subject in need thereof, the method comprising administering to the subject the CD161 binding protein of claim 1, to thereby deliver the CD161 binding protein to the subject.

72. A method of treating a proinflammatory in a subject in need thereof, the method comprising administering to the subject the CD161 binding protein of claim 1, to thereby treat the proinflammatory disease in the subject.

73. The method of claim 72, wherein the proinflammatory disease is multiple sclerosis, psoriatic arthritis, hidradenitis suppurativa, polyarticular juvenile arthritis, Sjogren's syndrome, psoriasis, alopecia areata, systemic sclerosis, one or more inflammatory bowel disease, ankylosing spondylitis, non-radiographic axial spondyloarthritis, systemic lupus erythematosus, discoid lupus erythematosus, rheumatoid arthritis, an idiopathic inflammatory myopathies, sarcoidosis, enteropathic arthritis, or palmoplantar pustulosis.

74. The method of claim 72, wherein the proinflammatory disease is an allergic disease, an eosinophilic gastrointestinal disorder, allergic rhinoconjunctivitis, or an IgE mediated disease.

75. The method of claim 72, wherein the proinflammatory disease is an autoimmune disease.

76.-102. (canceled)

103. A method of depleting a population of cells expressing CD161 in a subject in need thereof, the method comprising administering to the subject the CD161 binding protein of claim 1, to thereby deplete the population of cells expressing CD161 in the subject.

104.-113. (canceled)

114. A method of reducing the level of one or more proinflammatory cytokines in a subject in need thereof, the method comprising administering to the subject the CD161 binding protein of claim 1, to thereby reduce the level of one or more proinflammatory cytokines in the subject.

115.-126. (canceled)

127. A method of inhibiting binding of CD161 to CLEC2D expressed on the surface of a cell in a subject in need thereof, the method comprising administering to the subject the CD161 binding protein of claim 1, to thereby inhibit binding of CD161 to CLEC2D expressed on the surface of a cell in the subject.

128. A method of treating cancer in a subject in need thereof, the method comprising administering to the subject the CD161 binding protein of claim 1, to thereby treat the cancer in the subject.

129.-136. (canceled)
